SEQUENCE LISTING (32 sequences)

SEQ ID NO: 1 - DNA, 50 bp, synthetic construct
  source 1..50: mol_type = other DNA
attagcgaacgtggattttgtccgacatcggcaagctcactttttcgact

SEQ ID NO: 2 - DNA, 50 bp, synthetic construct
  source 1..50: mol_type = other DNA
ttgccgatgtcggacttttgaacgatcttcgcctgctgcgttttgtgagc

SEQ ID NO: 3 - DNA, 50 bp, synthetic construct
  source 1..50: mol_type = other DNA
aggcgaagatcgttcttttcctgcacgtccaactggtcagttttcgcagc

SEQ ID NO: 4 - DNA, 50 bp, synthetic construct
  source 1..50: mol_type = other DNA
agttggacgtgcaggtttttccacgttcgctaatagtcgattttctgacc

SEQ ID NO: 5 - DNA, 31 bp, synthetic construct
  source 1..31: mol_type = other DNA
ggaccatggttccacgttcgctaatagtcga

SEQ ID NO: 6 - DNA, 31 bp, synthetic construct
  source 1..31: mol_type = other DNA
tggtcaagcagaacgatcttcgcctgctgcg

SEQ ID NO: 7 - DNA, 31 bp, synthetic construct
  source 1..31: mol_type = other DNA
tcgactattagcgaacgtggaaccatggtcc

SEQ ID NO: 8 - DNA, 31 bp, synthetic construct
  source 1..31: mol_type = other DNA
cgcagcaggcgaagatcgttctagcttgacc

SEQ ID NO: 9 - DNA, 50 bp, synthetic construct; identical to SEQ ID NO: 1
  source 1..50: mol_type = other DNA

SEQ ID NO: 10 - DNA, 50 bp, synthetic construct; identical to SEQ ID NO: 2
  source 1..50: mol_type = other DNA

SEQ ID NO: 11 - DNA, 50 bp, synthetic construct; identical to SEQ ID NO: 3
  source 1..50: mol_type = other DNA

SEQ ID NO: 12 - DNA, 50 bp, synthetic construct; identical to SEQ ID NO: 4
  source 1..50: mol_type = other DNA

SEQ ID NO: 13 - DNA, 50 bp, synthetic construct; identical to SEQ ID NO: 1
  source 1..50: mol_type = other DNA

SEQ ID NO: 14 - DNA, 50 bp, synthetic construct; identical to SEQ ID NO: 3
  source 1..50: mol_type = other DNA

SEQ ID NO: 15 - DNA, 50 bp, synthetic construct; identical to SEQ ID NO: 1
  source 1..50: mol_type = other DNA

SEQ ID NO: 16 - DNA, 50 bp, synthetic construct; identical to SEQ ID NO: 3
  source 1..50: mol_type = other DNA

SEQ ID NO: 17 - DNA, 20 bp, synthetic construct
  source 1..20: mol_type = other DNA
tagtcgattttatccatgca

SEQ ID NO: 18 - DNA, 20 bp, synthetic construct
  source 1..20: mol_type = other DNA
tagtcgattttatccatgca

SEQ ID NO: 19 - DNA, 20 bp, synthetic construct
  source 1..20: mol_type = other DNA
tagtcgattttatccatgca

SEQ ID NO: 20 - DNA, 20 bp, synthetic construct
  source 1..20: mol_type = other DNA
tagtcgattttatccatgca

SEQ ID NO: 21 - DNA, 20 bp, synthetic construct
  source 1..20: mol_type = other DNA
tgcatggataaaatcgacta

SEQ ID NO: 22 - DNA, 25 bp, synthetic construct
  source 1..25: mol_type = other DNA
tgcatggataaaatcgactattttt

SEQ ID NO: 23 - DNA, 88 bp, synthetic construct
  source 1..88: mol_type = other DNA
acaggattttcgcctgctggggcaaaccagcgtggaccgcttttttggctattcttttgatttataagggattttgccgatttcggaa

SEQ ID NO: 24 - DNA, 88 bp, synthetic construct
  source 1..88: mol_type = other DNA
caactctctcagggccaggcggtgaagggcaatcagctgttgttttcaacagcatcctgtttccgaaatcggcattaaagaccagctg

SEQ ID NO: 25 - DNA, 88 bp, synthetic construct
  source 1..88: mol_type = other DNA
tctcactggtgaaaagaaaaaccaccctggcgcccaatacgctttttccccgcgcgttggccgattcattaatgcagctggcacgaca

SEQ ID NO: 26 - DNA, 88 bp, synthetic construct
  source 1..88: mol_type = other DNA
ggcgaaatgattgctttcaccagtgagatgtcgtgacgtggatttttccacgttctttaatagtggactcttgttccaaactggaaca

SEQ ID NO: 27 - DNA, 42 bp, synthetic construct
  source 1..42: mol_type = other DNA
tgttccaaatagccaagcggtccacgctccctgaggggcgcc

SEQ ID NO: 28 - DNA, 42 bp, synthetic construct
  source 1..42: mol_type = other DNA
cattaattttttctccttcaccgcctggggtttgcttataaa

SEQ ID NO: 29 - DNA, 42 bp, synthetic construct
  source 1..42: mol_type = other DNA
agggtgggaatcggacaagagtccactaaaatccccccagca

SEQ ID NO: 30 - DNA, 42 bp, synthetic construct
  source 1..42: mol_type = other DNA
tcaaaaggtttggaccaacgcgcggggagcgtattagagttg

SEQ ID NO: 31 - DNA, 66 bp, synthetic construct
  source 1..66: mol_type = other DNA
tgcatggataaaatcgactatttttgttccaaatagccaagcggtccacgctccctgaggggcgcc

SEQ ID NO: 32 - DNA, 66 bp, synthetic construct; identical to SEQ ID NO: 31
  source 1..66: mol_type = other DNA

(19) United States
(12) Patent Application Publication
(10) Pub. No.: US 2025/0052737 A1
(43) Pub. Date: Feb. 13, 2025

(54) TRIGGER-ASSEMBLED MEMBRANE-SPANNING NUCLEIC ACID NANOSTRUCTURES

(71) Applicant: UCL Business Ltd, London (GB)

(72) Inventors: Stefan HOWORKA, London (GB); Conor LANPHERE, London (GB)

(21) Appl. No.: 18/823,590

(22) Filed: Sep. 3, 2024

Related U.S. Application Data
(63) Continuation of application No. PCT/EP2023/055520, filed on Mar. 3, 2023.

(30) Foreign Application Priority Data
Mar. 3, 2022 (GB) 2202985.4

(51) Int. Cl. G01N 33/487; C12Q 1/6869

(52) U.S. Cl. CPC G01N 33/48721; C12Q 1/6869

(57) ABSTRACT

A nucleic acid nanostructure is provided that comprises a plurality of component modules, each component module comprising a nucleic acid sequence and at least one membrane anchor. The plurality of component modules are capable of undertaking a controlled assembly in response to an external stimulus to form the nanostructure and also to penetrate a semifluid membrane upon or following the controlled assembly. Methods of assembling the nanostructure as well as uses in sensors, drug delivery and release of imaging substances are also provided.

### CROSS-REFERENCE TO RELATED APPLICATIONS

This application is a continuation application of PCT/EP2023/055520, filed Mar. 3, 2023; which claims the priority of GB2202985.4, filed Mar. 3, 2022. The above applications are incorporated herein by reference in their entirety.

### REFERENCE TO SEQUENCE LISTING, TABLE OR COMPUTER PROGRAM

This application contains an ST.26 compliant Sequence Listing, which was submitted in xml format via Patent Center and is hereby incorporated by reference in its entirety. The .xml copy, created on Mar. 3, 2023 is named Seq Listing.xml and is 29,400 bytes in size.

### FIELD OF THE INVENTION

The present invention relates to novel nucleic acid nanostructures and their uses. In particular, it relates to nucleic acids that can act as dynamic functional membrane associated nanopores.

### BACKGROUND OF THE INVENTION

DNA nanostructures have shown potential to advance nanotechnology and the life sciences. Compared to other materials, DNA nanostructures have a highly controllable architecture which is based on predictable folding using base-pairing rules (Rothemund P. W. Nature 440, 297-302 (2006), Seeman, N. C.; Sleiman, H. F. Nat. Rev. Mater. (2017), 3, 17068; Hong, F. et al. Chem. Rev. 2017, 117, 12584-12640; Praetorius, F. et al. Nature (2017), 552, 84-87; Sacca, B.; Niemeyer, C. M. Angew. Chem. Int. Ed. 2012, 51, 58-66). By exploiting these properties, functional DNA nanostructures are increasingly designed to benefit areas outside DNA nanotechnology. Examples include DNA scaffolds which precisely position proteins and other biomolecular components for research applications in biophysics and molecular biology. Furthermore, predictable changes in DNA nanostructures have been exploited as smart biosensing devices which measure pH inside cells (Bhatia, D.; et al. Nat. Commun. (2011), 2, 339) or in cellular DNA nanocages for delivery of bioactive cargo (Walsh, A. S.; et al. ACS Nano (2011), 5, 5427-5432).

Replicating complex biological functions via simple and tuneable synthetic means is of considerable interest in science and technology. The myriads of biological nanopores and other membrane proteins are a powerful inspiration in this endeavour. By forming a water-filled channel, protein nanopores shuttle bioactive cargo across cell membranes and provide scientific insight into transport and molecular interaction within confined space. Such nanopores have found a use in portable and scalable DNA sequencing devices by allowing individual nucleic acid strands to pass a reading head. Nanopores are also used in sensing of non-DNA analytes. Reflecting these strengths, synthetic nanopores have been created with self-assembling peptides, organic molecules, or in solid state inorganic materials in order to broaden the sensing range and to understand how transport is influenced by pore chemistries, shapes and sizes not accessible in biology (see for example, Howorka, S. Nat. Nanotechnol. 2017, 12, 619-630; Xue, L., et. al. Nat. Rev. Mater. 2020, 5, 931-951; and Wei, R. S. et al. Nat. Nanotechnol. 2012, 7, 257-263).

Conventional configurations of membrane-spanning nanopores are, however, typically constitutively open, which limits their functional complexity. To address this constraint, barrel-like pores have been equipped with a lid that can be removed or opened in response to an external stimulus. Yet, such pores are rarely ever fully closed and as long as the nanostructure penetrates and spans the membrane this allows for a small amount of current flow from the cis to trans sides of the membrane, or even transversely across the membrane which can affect sensing applications. In addition, the presence of a constitutively open pore lumen also permits leakage of cargo even in the nominally closed state or cause further leakage during the process of insertion into bilayers and related semifluid membranes. It will be appreciated that leakiness can reduce the pores' application potential in analyte sensing—where it contributes to increased background noise—as well as in drug delivery, or targeted cell lysis.

There is a need to further develop further improved and optimized methods and compositions for assembly and insertion of nanopores into bilayers and semi-fluid membranes. The present invention addresses the deficiencies in the art. These and other uses, features and advantages of the invention should be apparent to those skilled in the art from the teachings provided herein.

### SUMMARY OF THE INVENTION

In various embodiments, the invention provides DNA nanotechnology to construct a functionally advanced membrane pore that assembles from a plurality of membrane surface-associated subunits following a defined triggered activation. The controlled formation of the nanopore integrates the processes of molecular recognition between the triggers and the inactive subunits, the repositioning of the activated subunits within the membrane, and their assembly into a functional membrane-spanning channel.

A first aspect of the invention provides a nucleic acid nanostructure comprising:
a plurality of component modules, each component module comprising a nucleic acid sequence and at least one membrane anchor, and
wherein the plurality of component modules are capable of undertaking a controlled assembly in response to an external stimulus to form the nanostructure; and
wherein the nanostructure is configured to penetrate a semifluid membrane upon or following the controlled assembly.

Optionally, the plurality of component modules are able to associate with a semifluid membrane prior to or following the controlled assembly. Suitably, the nanostructure is configured to penetrate the semifluid membrane upon, during or following the controlled assembly

A second aspect of the invention provides a nucleic acid nanostructure comprising:
a plurality of component modules, each component module comprising a deoxyribonucleic acid (DNA) sequence, wherein the DNA sequence comprises at least a portion of double helix that defines a secondary structure and at least a portion of single stranded sequence that defines an assembly interface, and at least one hydrophobic anchor; and
a plurality of single stranded nucleic acid lock sequences that are capable of hybridising with the single stranded sequence of the assembly interface;
wherein the component modules are able to associate and interact with a surface of a semifluid membrane via the anchor, and
wherein the modules are capable of undertaking a controlled assembly in response to an externally applied stimulus that initiates disassociation of the plurality of lock sequences to reveal the assembly interfaces thereby allowing the plurality of component modules to associate and thereby form a nanostructure.

In a specific embodiment, the component modules are configured to associate and interact with a surface of a semifluid membrane via the anchor.

A third aspect of the invention provides a semifluid membrane onto which is associated a nucleic acid nanostructure as described in any one of the embodiments set out herein.

A fourth aspect of the invention provides for a semifluid membrane onto which is associated a plurality of nanostructure component modules, each component module comprising a deoxyribonucleic acid (DNA) sequence, wherein the DNA sequence comprises at least a portion of double helix that defines a secondary structure and at least a portion of single stranded sequence that defines an assembly interface, and at least one hydrophobic anchor; and a plurality of single stranded nucleic acid lock sequences that are capable of hybridising with the single stranded sequence of the assembly interface.

In a specific embodiment the semifluid membrane comprises a synthetic amphipathic membrane or a lipid bilayer membrane.

A fifth aspect of the invention provides a sensor device that comprises a nucleic acid nanostructure as described in any one of the embodiments set out herein.

A sixth aspect of the invention provides a sensor device, wherein the sensor device comprises a semifluid membrane as defined in any one of the embodiments set out herein.

A seventh aspect of the invention provides a semifluid membrane vesicle, wherein the vesicle comprises a nucleic acid nanostructure as defined herein.

An eighth aspect provides a pharmaceutical composition comprising a semifluid membrane vesicle as defined herein, and a pharmaceutically acceptable carrier.

A ninth aspect of the invention provides for a method for assembly of a nucleic acid nanostructure, the method comprising:
providing a plurality of component modules, each component module comprising
a nucleic acid sequence, wherein the nucleic acid sequence comprises at least a portion of double helix that defines a secondary structure and at least a portion of single stranded sequence that defines an assembly interface, wherein the assembly interface of each of the plurality of component modules comprises a sequence that is complementary to and is capable of hybridising with the assembly interface of another of the component modules; and
at least one hydrophobic anchor;
   and, combining the plurality of component modules under conditions that allow for the spontaneous assembly of the nanostructure.

A tenth aspect of the invention provides a method for triggered assembly of a nucleic acid nanostructure, the method comprising:
providing a plurality of component modules, each component module comprising a nucleic acid sequence, wherein the nucleic acid sequence comprises at least a portion of double helix that defines a secondary structure and at least a portion of single stranded sequence that defines an assembly interface, wherein the assembly interface of each of the plurality of component modules comprises a sequence that is complementary to and is capable of hybridising with the assembly interface of another of the component modules, and at least one hydrophobic anchor,
providing a plurality of single stranded nucleic acid lock sequences that hybridise with the single stranded sequence of the assembly interface, thereby forming a plurality of locked component modules that are inhibited from initiating spontaneous assembly into a nucleic acid nanostructure;
exposing the plurality of locked component modules to an external stimulus, wherein the external stimulus initiates dissociation of the plurality of single stranded nucleic acid lock sequences from the plurality of locked component modules thereby exposing the plurality of assembly interfaces; and
providing conditions that allow for the spontaneous assembly of the nanostructure.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

One or more embodiments of the invention will now be described, by way of example, with reference to the accompanying drawings, in which:

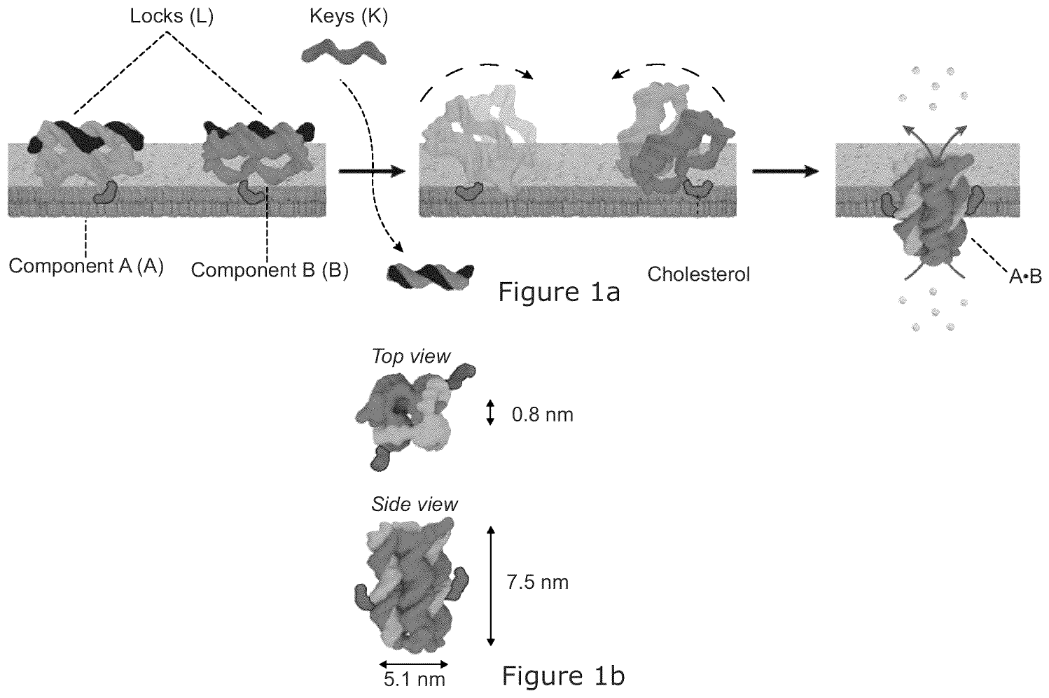

FIGS. 1a-1b show triggered assembly of a nanostructure in the form of a DNA nanopore A·B from components A and B at the membrane interface. (a) Assembly-inactive components ALA and BLB carrying locks LA and LB are tethered to the membrane via a cholesterol anchor (grey crescent shape). Addition of key strands KA and KB, unzips the lock strands to allow activated A and B to reoriented relative to the bilayer and form a membrane spanning ion channel. (b) Top-down and side views of the A·B pore featuring four DNA helices arranged in a square lattice. The membrane cholesterol anchors are embedded and curving into the lipid bilayer.

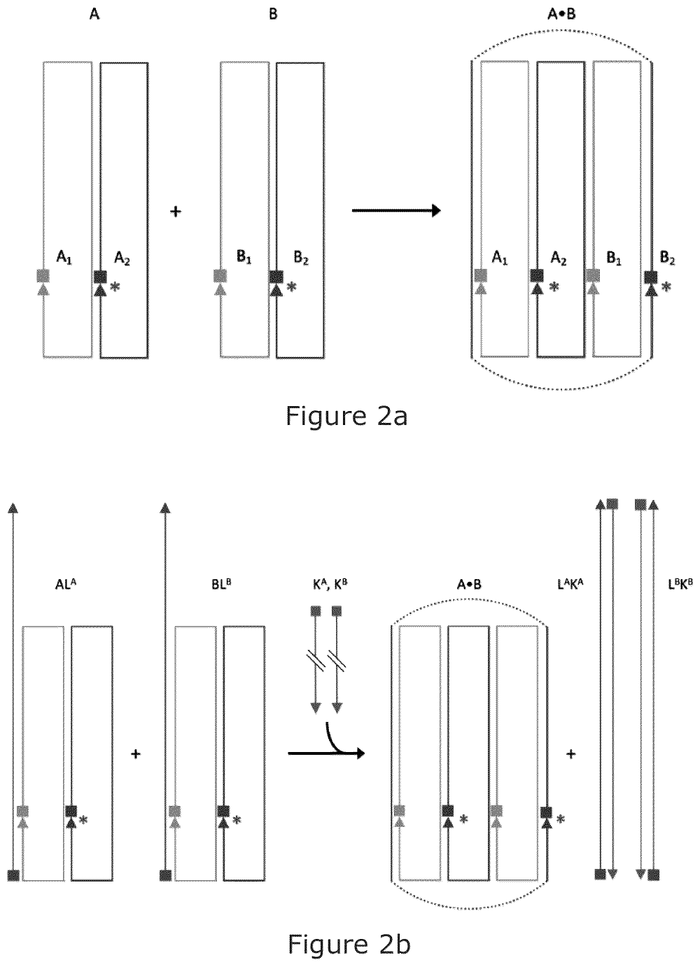

FIGS. 2a-2b are a representation of an embodiment of the invention that shows 2D strand maps of direct and triggered assembly of A·B. (a) 2D strand map schematic the components A and B and assembly into the 4HB nanopore, A·B. (b) 2D strand map schematic of the assembly locked components AL^{A} and BL^{B} indicating the mechanism of assembly into A·B upon addition of the keys (K^{A} and K^{B}) to remove the locks (L^{A} and L^{B}). The resultant lock-key duplexes are indicated at the right. Squares represent the 5′ and triangles the 3′ end of DNA, asterisks represent cholesterol anchor modifications attached to the 3′ end of the indicated strand via a tri(ethylene)glycol (TEG) linker. Strands that make up the pore are shown in alternating light grey and dark grey. The crossovers at the top and bottom of each strand consist of four thymine bases each.

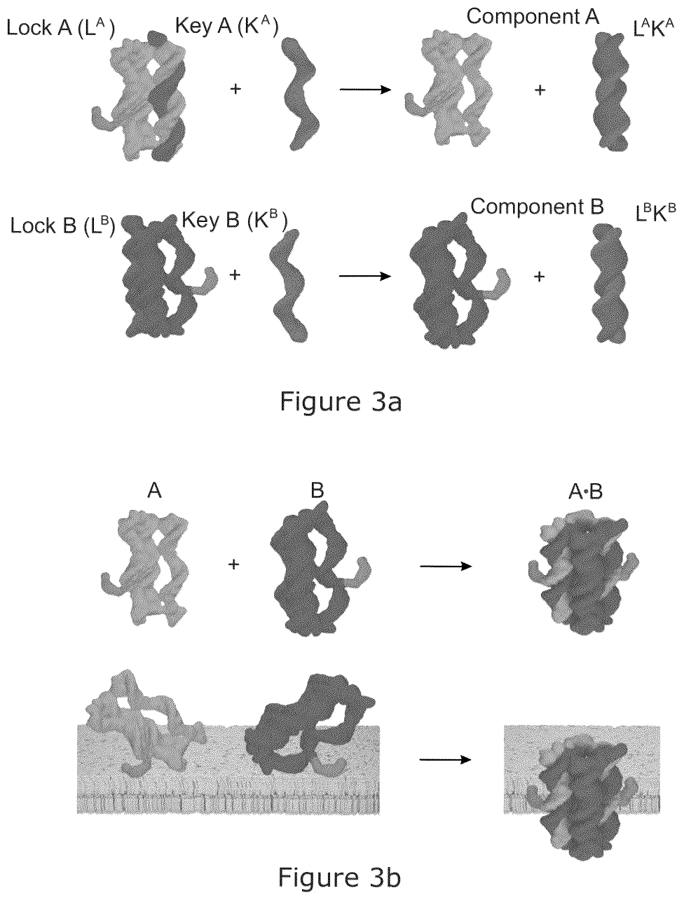

FIGS. 3a-3b show a schematic illustrating the triggering mechanism and assembly in solution and on membranes. (a) Assembly locked pre-pore components (AL^{A} and BL^{B}) are composed of pore components (A and B) plus a lock strands (L^{A} and L^{B}), respectively, which prevent pore formation. Addition of key strands (K^{A} and K^{B}) unzip each lock strand via a toehold mechanism leaving the unlocked components (A and B). (b) A and B are free to self-assemble in solution (top) or on the surface of the membrane (bottom) to form active ion channels.

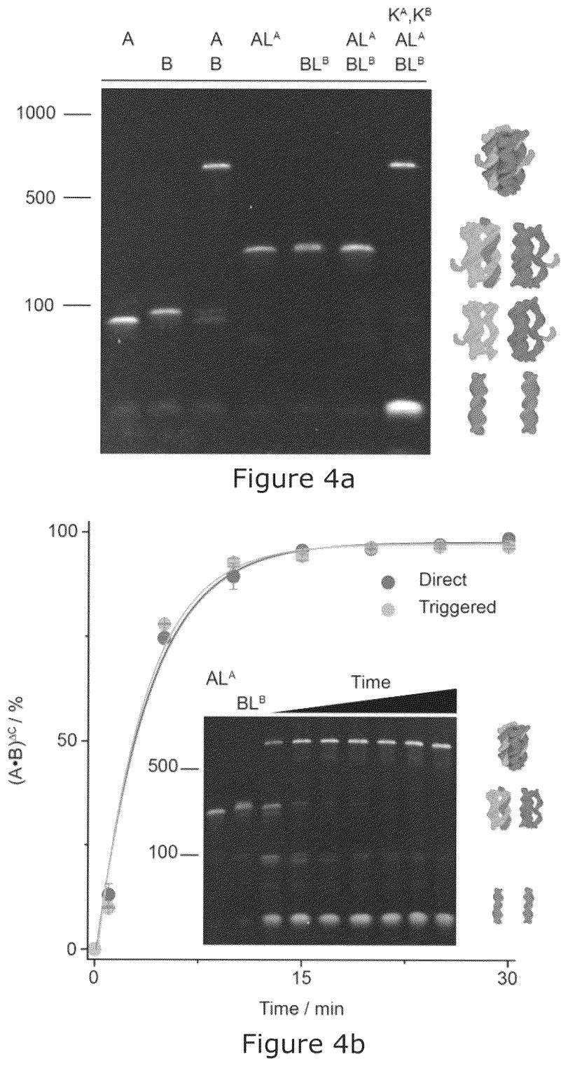

FIGS. 4a-4b show results that demonstrate that nanostructure A·B forms by direct and triggered assembly. (a) polyacrylamide gel electrophoresis (PAGE) analysis of A·B formation by direct assembly from components A and B (lanes 1-3) and triggered assembly of AL^{A} and BL^{B}(lanes 4-7) either in the absence or presence of key strand K^{A} and K^{B} (lanes, 4-6, and 4, 5, 7, respectively). (b) PAGE-based kinetic analysis of (A·B)^{ΔC} formation by direct (darker line) and triggered (lighter line) mechanisms. A representative PAGE gel for triggered assembly is shown as an inset.

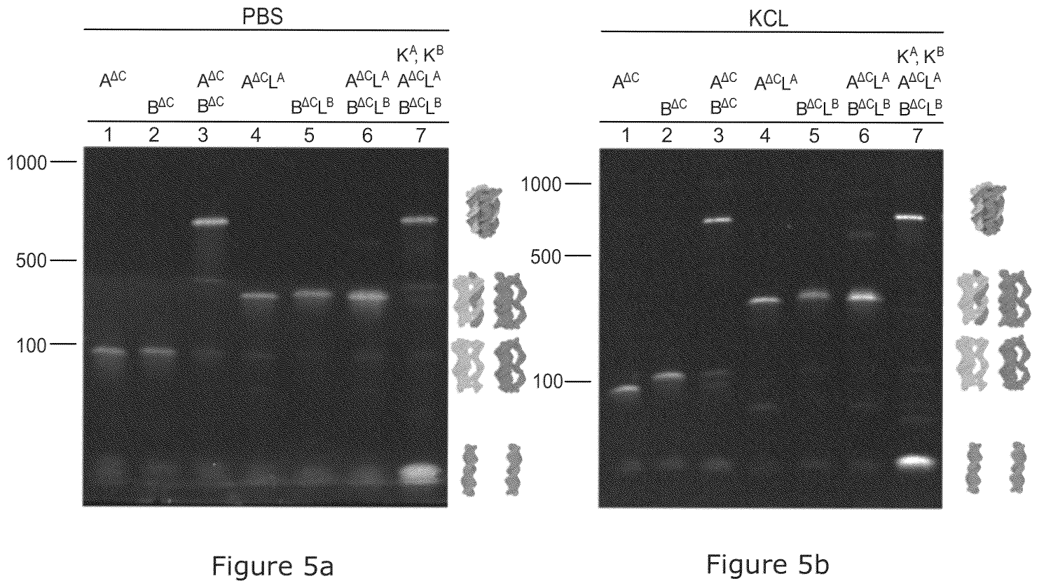

FIGS. 5a-5b show results of PAGE characterization of A·B pore formation. Confirmation of the assembly of the nanopore (A·B)^{ΔC} from component parts in PBS (left) and KCl (300 mM KCl, 15 mM Tris-HCl, pH 7.4; right). Assembly of (A·B)^{ΔC} from unlocked components and the efficacy of the locking mechanism to control and trigger pore assembly was assessed by native PAGE. Addition of A^{ΔC} and B^{ΔC} in a 1:1 stoichiometric ratio results in the assembly of (A·B)^{ΔC} (Lanes 1-3). Addition of A^{ΔC}L^{A}+B^{ΔC}L^{B} in a 1:1 stoichiometric ratio show no interaction and remain in their constituent parts (Lanes 4-6). However, addition of keys K^{A} and K^{B} in a 1:1 stoichiometric ratio removes the locks and results in the formation of (A·B)^{ΔC} (Lane 7). Addition of keys in a 1:1 stoichiometric ratio is also sufficient for complete removal of the corresponding lock (Lane 7). Samples were incubated at 30° C. for 30 min before being loaded onto the gel. 10% (left) and 12% (right) native PAGE were run in 1×TBE buffer at 90 V for 115 min at 4° C.

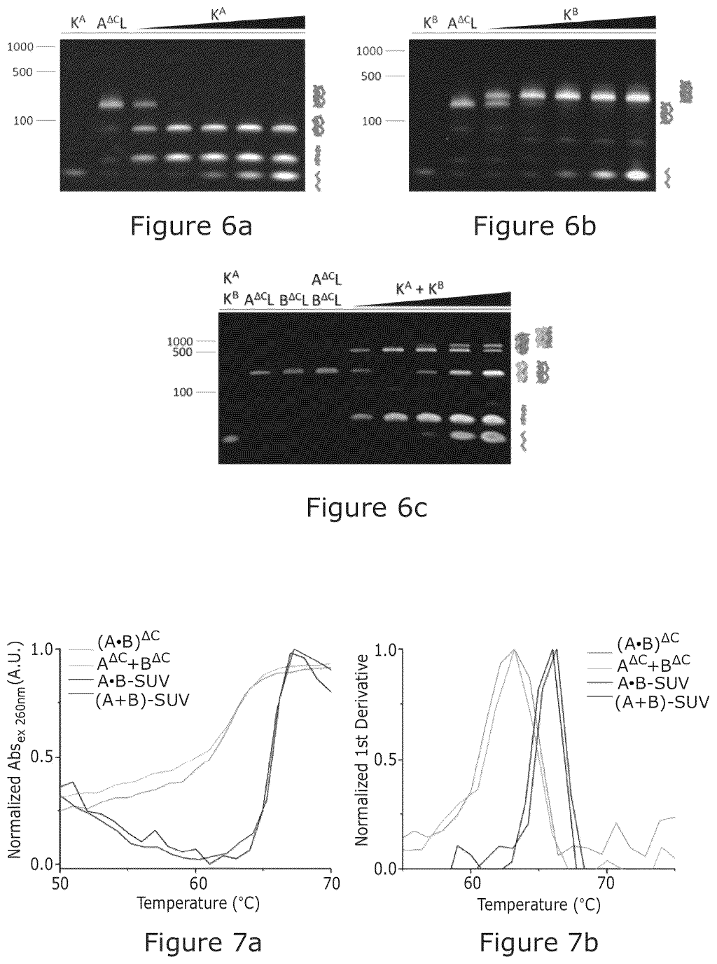

FIGS. 6a-6c show results of a gel electrophoretic mobility shift assay on the key specificity and the concentration dependence of triggered pore assembly. (a) Key K^{A} was mixed with assembly-locked component A^{ΔC}L^{A} at molar ratios ranging from 1:0.5 to 1:10 of A^{ΔC}L:K^{A}. The gel indicates that A^{ΔC}L^{A} is quantitatively converted to A^{ΔC} at a stoichiometric ratio of 1:1. Excess key does not interact with A^{ΔC}. (b) Key K^{B} was added to assembly locked component A^{ΔC}L^{A} at molar ratios ranging from 1:0.5 to 1:10 A^{ΔC}L:K^{B}. The gel indicates that A^{ΔC}L^{A} is not unlocked and instead forms a new structure A^{ΔC}L^{A}K^{B} at a stoichiometric ratio of 1:1 and that excess key does not interact further once a stoichiometric structure has been formed. (c) Keys, K^{A} and K^{B} were mixed with the assembly locked components A^{ΔC}L^{A} and B^{ΔC}L^{B} in molar ratios of 1:0.5 to 1:10 of A^{ΔC}L^{A}, B^{ΔC}L^{B}:K^{A}, K^{B}. The gel indicates that when all four elements are at a 1:1:1:1 stoichiometric ratio that the pore (A·B)^{ΔC} and the two duplexes K^{A}L^{A} and K^{B}L^{B} are the sole products. As the amount of keys increases, other products such as the assembly-incomplete A^{ΔC}B^{ΔC}D emerge, where D=L^{A}, L^{B}, K^{A}, or K^{B}. At a 1:1:10:10 stoichiometric ratio the keys inhibit pore formation resulting in a significant amount of assembly-locked A^{ΔC}L^{A} and B^{ΔC}L^{B}, free keys and minor amounts of product A^{ΔC}B^{ΔC}D as well as the assembled (A·B)^{ΔC}.

FIGS. 7a-7b show graphs of UV-melting profiles. (a) UV-melting profiles of nanostructure A·B when pre-annealed (light blue) or directly assembled from A and B (light red). The melting profiles were assessed in solution or when inserted into small unilamellar vesicles (SUVs) composed of DPhPC lipids with a diameter of 170 nm. A·B was assessed at 0.2 μM with 200 μM DPhPC lipid in 150 μL of PBS buffer. Samples were melted using a temperature ramp of 1° C./min from 20 and 80° C. Each trace represents an average from three independent repeats. (b) 1^{st} derivative of the melting profiles used to identify the melting temperature (Tm) of each structure.

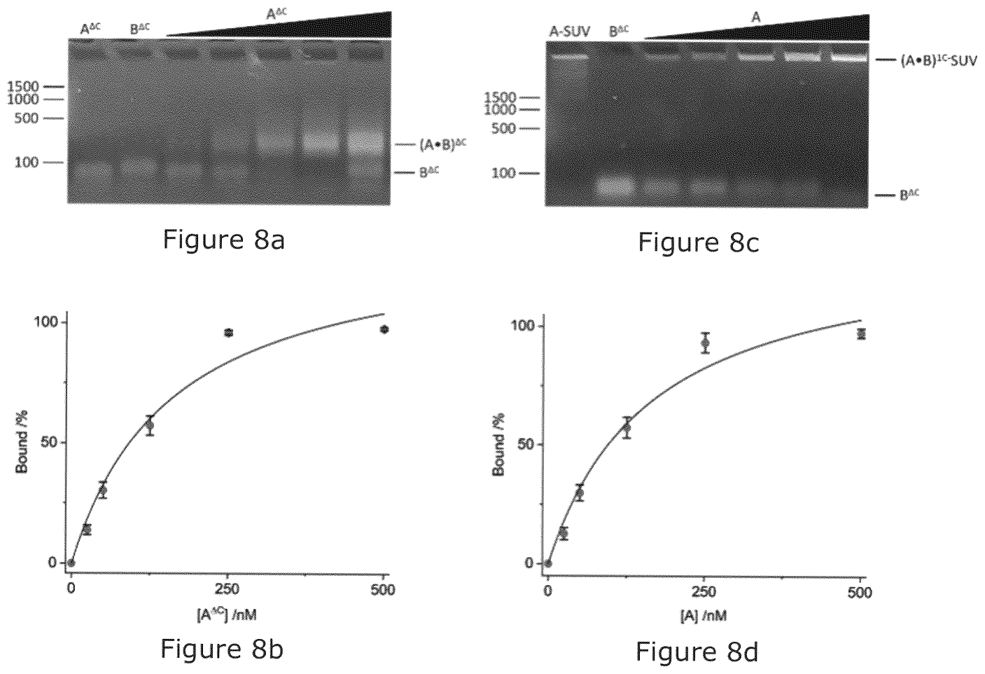

FIGS. 8a-8d show the results of a gel electrophoretic mobility shift assay on the formation of the A·B nanostructure. (a) A representative 3% agarose gel demonstrating the binding of AΔC and BΔC in solution. Addition of increasing concentrations of AΔC results in the formation of the (A·B)ΔC nanopore complex, which is larger than the individual components and migrates through the gel more slowly. (b) Binding curve of (A·B)ΔC complexation fit to a Langmuir curve revealed a Kd of 1.54±0.23×10-7 M. The data represent averages and standard deviations from three independent experiments. (c) A representative 2% agarose gel demonstrating binding of A-SUV and BΔC on the membrane surface. Addition of increasing concentrations of A results in the formation of the (A·B)1C membrane-bound complex, which is unable to migrate into the gel matrix and remains in the well. (d) Binding curve of (A·B)1C complexation. Fitting to a Langmuir curve revealed a Kd of 1.64±0.14×10-7 M. The data represent averages and standard deviations from three independent experiments. All gels were run in 1×TAE buffer at 60 V for 60 min. The major markers of a 100 bp DNA ladder are indicated on the left of each gel. Bands are identified at the top and right.

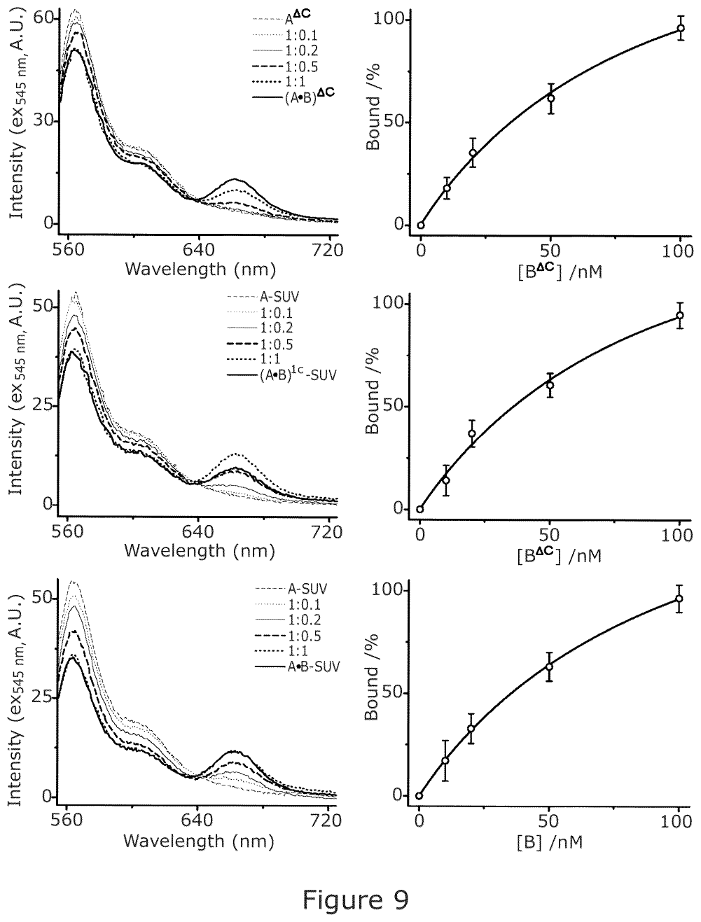

FIG. 9 shows graphs of results of Förster resonance energy transfer (FRET) analysis on nanostructure assembly. Components A and B carry the FRET pair Cy3 and Cy5, respectively. Increasing concentrations of ^{Cy5}B (FRET acceptor) are added to ^{Cy3}A (FRET donor) resulting in assembly into ^{Cy3}A·^{Cy5}B leading to close proximity and FRET, which is manifest as a decrease in Cy3 emission intensity and an increase in Cy5 emission intensity. (a) Pore assembly titration of A^{ΔC} vs B^{ΔC} in solution. Ratios are of A^{ΔC}:B^{ΔC} (b) Binding curve of (A·B)^{ΔC} pore assembly based on (a) displayed as percent bound. (c) Pore assembly titration of the membrane tethered A-SUV vs B^{ΔC}. Ratios are of A:B^{ΔC} (d) Binding curve of (A·B)^{1C} pore assembly based on (c) displayed as percent bound. (e) Pore assembly titration of A vs B on the membrane surface. Ratios are of A:B (f) Binding curves of A·B pore assembly based on (e) displayed as percent bound. The data displayed in (b, d, and f) represent averages and standard deviations from three independent experiments.

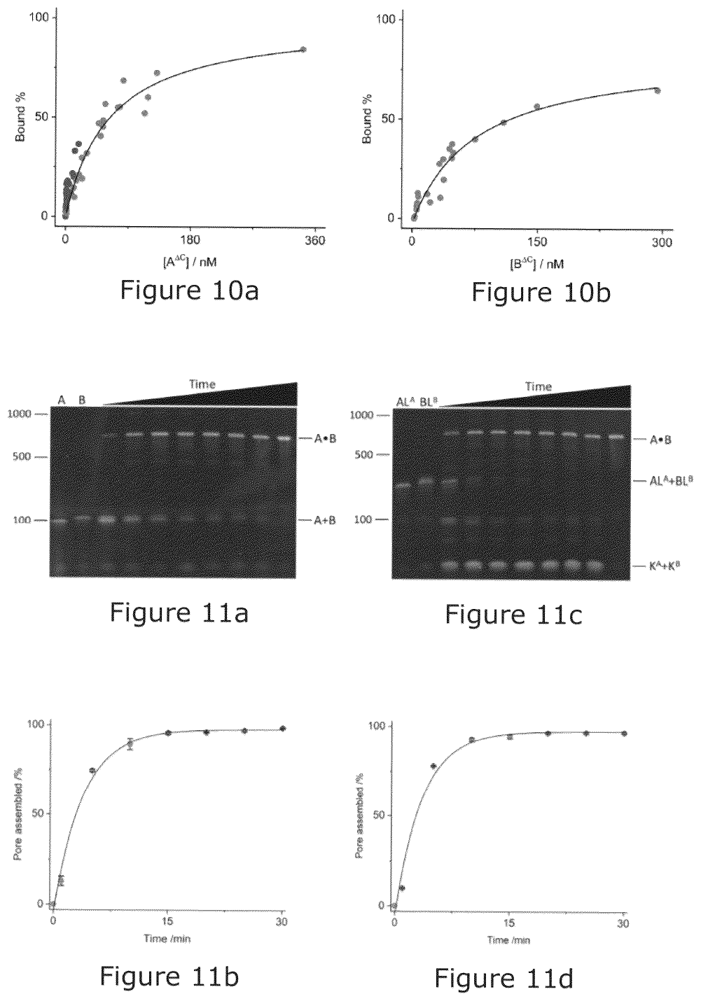

FIGS. 10a-10b show Dual-colour fluorescence cross-correlation spectroscopy analysis of pore assembly. Components A^{ΔC} and B^{ΔC} carried an Alexa488 and an Alexa647 fluorophore, respectively. (a) Example binding curve of (A·B)^{ΔC} pore formation as a function of ^{Alexa488}A^{ΔC} concentration (grey dots). The concentration of ^{Alexa647}B^{ΔC} was held constant at 16 nM. Each point represents a separate sample measurement. The solid line represents a Langmuir isotherm that was fitted to the data, yielding a K_{d}=6.39±1.07×10⁻⁸ M. The average of both repeats yielded a Kd of 7.17±0.77×10⁻⁸ M with maximum observed binding of A=101%. The clustered grey dots to the left represent the same experiment, but with ^{Alexa647}B^{ΔC} held constant at 1 nM. (b). Example binding curve of (A·B)^{ΔC} pore formation as a function of ^{Alexa647}B^{ΔC} concentration (grey dots). The concentration of ^{Alexa488}A^{ΔC} was held constant at 15 nM. The solid line represents a Langmuir isotherm that was fitted to the data, yielding a K_{d}=7.99±1.68×10⁻⁸ M. The average of three repeats resulted in a Kd of 6.22±1.25×10⁻⁸ M and maximum observed binding of B=84%.

FIGS. 11a-11d show a PAGE kinetic electrophoretic mobility shift assays of direct and triggered pore assembly. (a) PAGE titration to assess A·B formation from the direct assembly of A^{ΔC}+B^{ΔC}. (b) Quantitative analysis of the kinetic data from (a) via plotting the normalized amount of assembled A·B against time. The amount of assembled A·B was extracted from the gel band brightness as 1−(I_{A}−I_{background}) where I=intensity. (c) PAGE titration to assess A·B assembly from the triggered assembly of AL^{A}+BL^{B}+K^{A}+K^{B}. (d) Quantitative analysis of the kinetic data from (a) via plotting the normalized amount of assembled A·B against time. The amount of assembled A·B was extracted from the gel band brightness as 1−(I_{AL}−I_{background}) where I=intensity. 12% native PAGE run in 1×TBE buffer at 115 V for 90 min at 4° C. Time points are 1, 5, 10, 15, 20, 25, and 30 min. Each trace represents data from three independent repeats. The major markers of a 100 bp DNA ladder are indicated at the left.

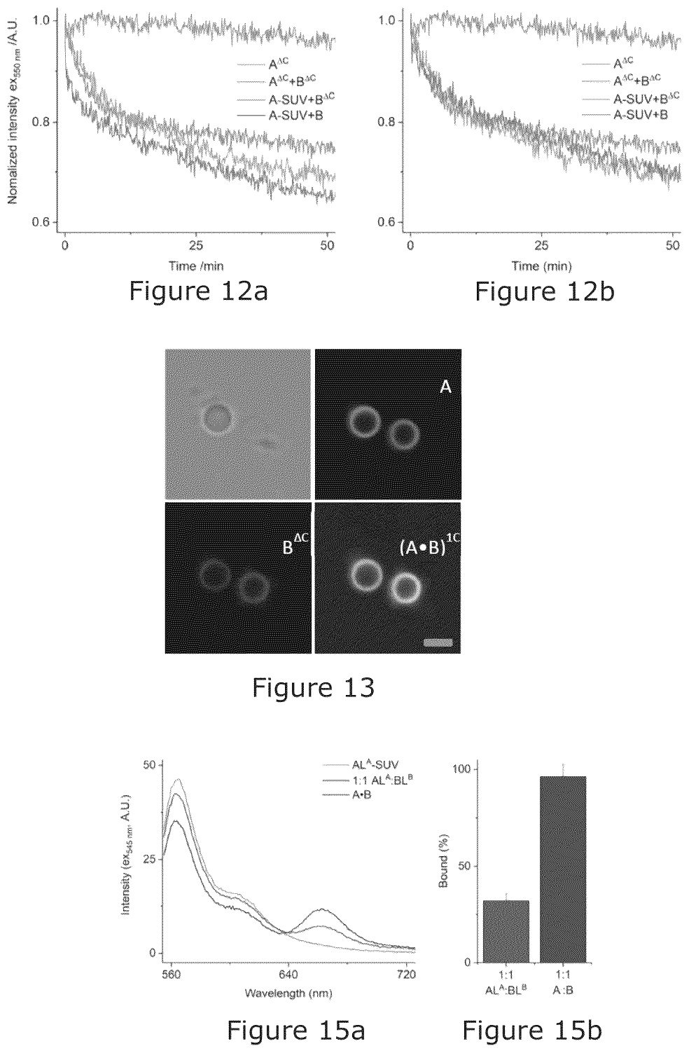

FIGS. 12a-12b show nanostructure assembly kinetics monitored via FRET. (a) Kinetic fluorescence analysis of pore assembly over time in solution (A^{ΔC}+B^{ΔC}, red), when one component is tethered to the membrane (A-SUV+B^{ΔC}, blue), and on the membrane surface (A-SUV+B, purple). A control trace (grey) was also added using A^{ΔC} and buffer only. (b) The same as (a), but the initial rapid decline of A-SUV+B (purple) has been removed. Each trace is an average of three independent repeats.

FIG. 13 shows confocal microscopy images that confirm cholesterol-mediated membrane tethering of (A·B)1C to GUVs and nanostructure assembly on the membrane surface. Component A was modified with a Cy3 fluorophore and B with a Cy5 fluorophore. Images were obtained using a 96× oil objective, scale bar 5 μm. (Top left) Brightfield image of two GUVs composed of POPC lipids. (Top right) Green channel shows a ring around the GUVs demonstrating the Cy3A tethering to the membrane. (Bottom left) Red channel shows Cy5BΔC, which cannot interact with the membrane on its own, has bound to the membrane tethered component A. (Bottom right) Overlay of the red and green channels demonstrating (A·B)1C formation and membrane tethering.

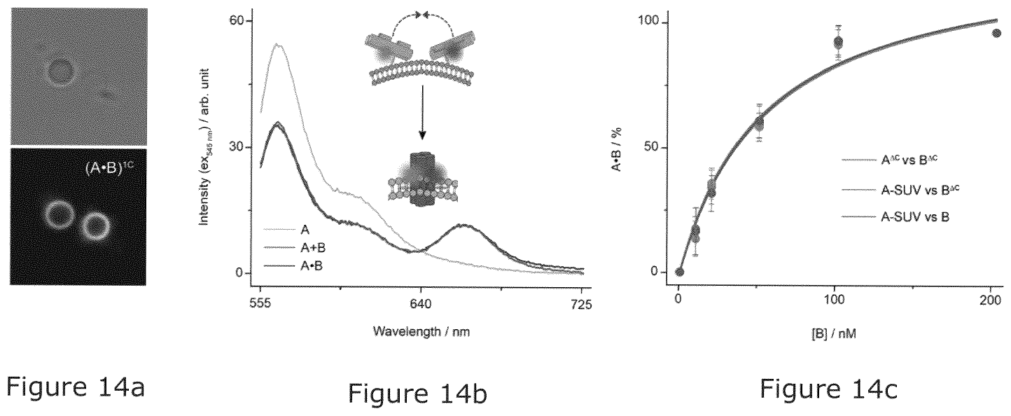

FIGS. 14a-14c show assembly of a nanostructure, in the form of a DNA nanopore, A·B from fluorophore-labeled components. ^{Cy3}A and ^{Cy5}B at the membrane interface analyzed using fluorescence microscopy and Förster resonance energy transfer (FRET). (a) Confocal microscopy images of the tethering of (A·B)^{1C} to a GUV composed of POPC lipids. Brightfield image (top) and overlay (bottom) of Cy5 (red) and Cy3 (green) channels. (b) Emission spectra for pore assembly on SUVs composed of DPhPC lipids, using excitation at 545 nm. The ^{Cy3}A emission peak at 563 nm (pink) drops when ^{Cy5}B is added at a 1:1 ratio (grey) to the same level as for the pre-annealed A·B pore (dark grey). (c) Concentration-dependent Cy3-emission change indicating pore assembly in solution (A^{ΔC} vs B^{ΔC}) and at the membrane interface (A-SUV vs B^{ΔC}, and A-SUV vs B). (n=3).

FIGS. 15a-15b show graphs that present the results of experiments demonstrating FRET control between assembly-locked components at the membrane surface. (a) Fluorescence spectra of the assembly locked components AL^{A} and BL^{B} to confirm pore assembly. The control also serves to assess the effect of Cy3-Cy5 proximity on the membrane surface. (b) Normalized bar graph comparing the FRET signal observed from a 1:1 stoichiometric ratio of assembly locked components, AL^{A} and BL^{B}, on the membrane surface to FRET signal observed from the direct assembly of A+B in a 1:1 stoichiometric ratio. Each trace is an average of three independent repeats, the standard deviation shown as the error bars in the bar chart.

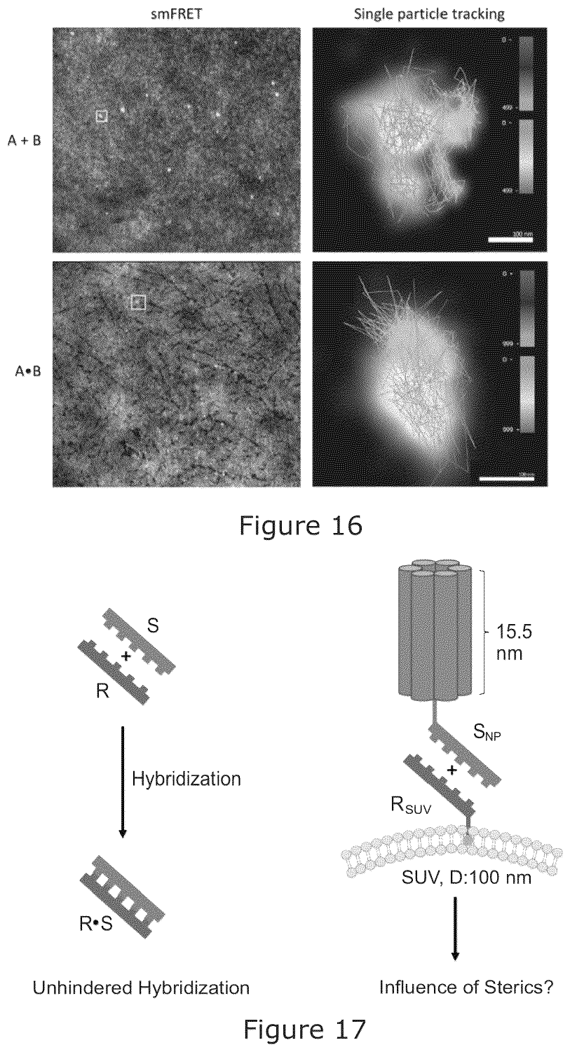

FIG. 16 shows smFRET and single particle tracking confirm A+B assembly at the membrane surface. Single-molecule FRET (smFRET) and single particle tracking images confirm nanostructure assembly from the monomer component A and B (top) in comparison to a control pre-mixed A·B (bottom). Bright green spots indicate the formation of A·B in the smFRET images (e.g., grey boxes). A carries a Cy3 fluorophore at the 5′ end of strand A₁ and B carries a Cy5 fluorophore at the 5′ end of strand B₁. Pore assembly from A and B was monitored on the supported lipid bilayer membrane at 62.5 μM. Single particle tracking experiments were monitored for 500 and 1000 s for A+B and pre-mixed A·B, respectively.

FIG. 17 shows a schematic illustration of the model system used to investigate the effect of sterics on DNA hybridization. Schematic illustrating the model system used to probe the influence of sterics on DNA hybridization on membrane surfaces. The simplest of the four conditions, hybridization of the receptor (R) and the ligand, (S) is shown on the left. The most sterically hindered of the four conditions, hybridization of the vesicle anchored receptor (R_{SUV}) and the ligand attached to the nanopore, (S_{NP}) is shown on the right. The cholesterol lipid anchors are shown in orange inserted into a grey lipid bilayer. The SUVs used were composed of DOPC and DOPE in a 7:3 mole ratio and extruded to be ˜100 nm in diameter. Only two of the four conditions are shown for clarity.

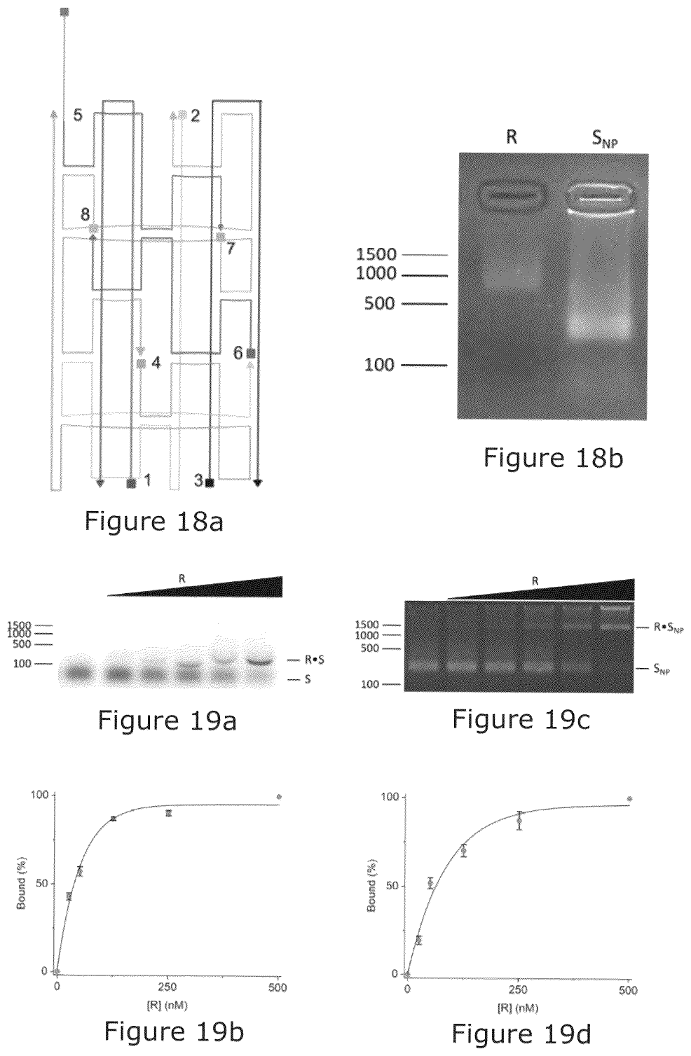

FIGS. 18a-18b show structure and assembly confirmation of S_{NP}. (a) 2D strand map of S_{NP}. All strands shown are numbered. Squares represent the 5′ end of DNA and circles represent the 3′ end of DNA. (b) 2% agarose gel confirming the presence of R and the assembly of S_{NP}.

FIGS. 19a-19d show the results of a gel electrophoretic mobility shift assay to assess the binding of the receptor (R) to the ssDNA ligand (S) or nanopore-bound ligand (S_{NP}). (a) A 3% agarose gel shift assay. Increasing concentrations of R were added to S resulting in the formation of the R·S dsDNA complex, which manifests as a more slowly migrating band. S carries a Cy5 fluorophore to enable imaging at low concentrations. (b) Binding curve of R·S complexation displayed as percent bound. The data were fit with a Langmuir binding curve and represent averages and standard deviations from 3 independent experiments. (c) A 2% agarose gel shift assay measuring the formation of the R·S_{NP} complex. For improved separation, R modified with a cholesterol lipid anchor was sued. While the S_{NP} is significantly more massive than R, the cholesterol modification interacts with the gel matrix resulting in significantly reduced R·S_{NP} mobility. (d) Binding curve of R·S_{NP} complexation displayed as percent bound. The data were fit with a Langmuir binding curve and represent averages and standard deviations from 3 independent experiments. All gels were run in 1×TAE buffer at 60 V for 60 min. The major markers of a 100 bp DNA ladder are indicated on the left.

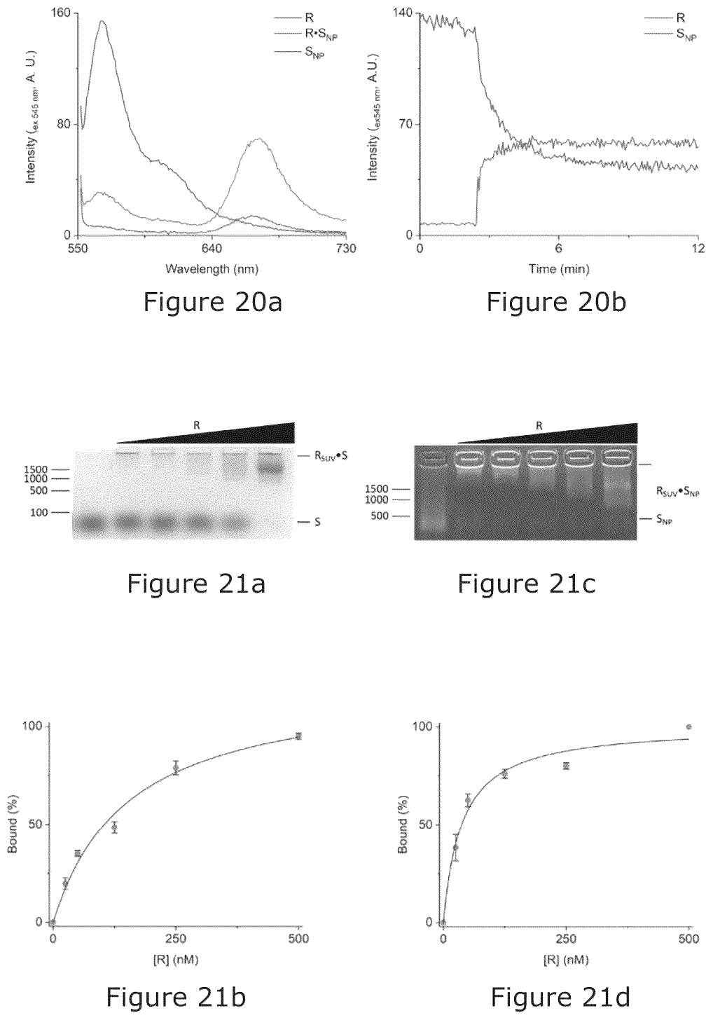

FIGS. 20a-20b show the results of a fluorescence analysis of the interaction of R and S_{NP} labeled with the FRET pair, Cy3 and Cy5, respectively. (a) Ensemble fluorescence measurement of the hybridization of R (black) and S_{NP} (blue) resulting in complex R·S_{NP} (red confirmed via FRET. (b) Kinetic fluorescence measurement of the change in intensity of ^{Cy3}R and ^{Cy5}S_{NP} as a result of hybridization overtime. Measured in a 1:1 stoichiometric ratio of ^{Cy3}R:^{Cy5}S_{NP}

FIGS. 21a-21d show results of a gel electrophoretic mobility shift assay to assess the binding of the ssDNA ligand (S) or nanopore-bound ligand (S_{NP}) to the receptor (R), which is tethered to the surface of SUVs. (a) A 3% agarose gel shift assay. Addition of increasing concentrations of R results in the formation of the R_{SUV}·S membrane-bound dsDNA complex, which is unable to migrate into the gel matrix and remains in the well. S carries a Cy5 fluorophore to improve resolution at low concentrations. The gel was imaged using a fluorescence gel scanner. (b) Binding curve of R_{SUV}·S complexation displayed as percent bound. The data are fit with a Langmuir binding curve and represent averages and standard deviations from 3 independent experiments. (c) A 2% agarose gel shift assay: increasing the concentration of R results in the formation of the R_{SUV}·S_{NP} membrane-bound complex, which is unable to migrate into the gel matrix and remains in the well. (d) Binding curve of R_{SUV}·S_{NP} complexation displayed as percent bound. The data were fit with a Langmuir binding curve and represent averages and standard deviations from 3 independent experiments. All gels were run in 1×TAE buffer at 60 V for 60 min. The major markers of a 100 bp DNA ladder are indicated on the left.

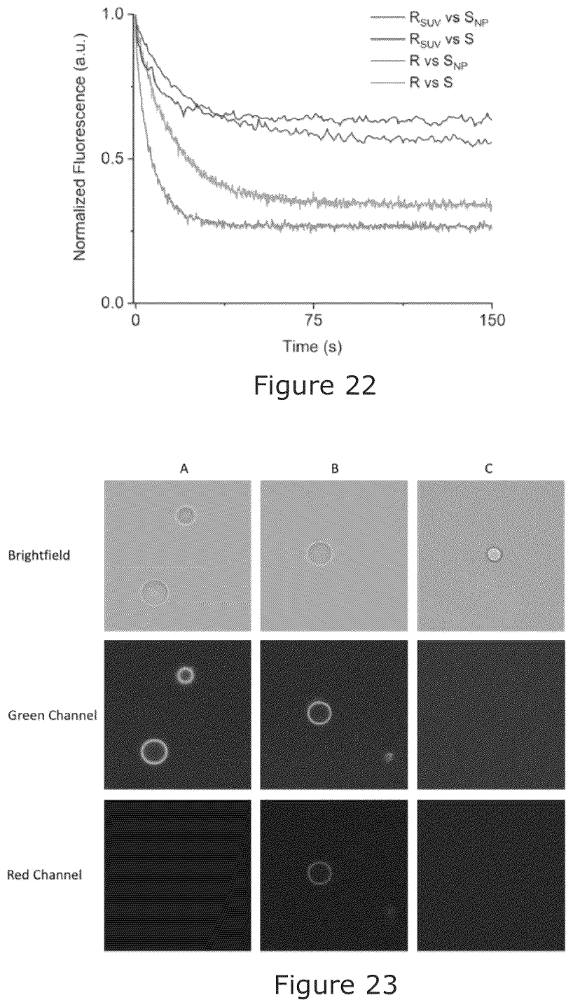

FIG. 22 is a graph showing FRET kinetic analysis of hybridization between R and S (or S_{NP}) in solution and on the membrane surface. The fluorescence signal was normalized as F/F₀ from the point of S or S_{NP} addition. For R+S (bottom line) and R+S_{NP} (second from bottom line), R was added at 1.25 nM and S and S_{NP} were added in a 100-fold excess at 125 nM. R_{SUV} vs S (top line at 150 s) and R_{SUV} vs S_{NP} (second from top line at 150 s), R was added at 5 to nM and S and S_{NP} were added in a 20-fold excess at 100 nM. SUVs (100 nm) were added at a concentration of 22.2 μM for an R:SUV loading of 225:1 or a lipid:R ratio of 1167:1.

FIG. 23 shows Confocal microscopy images of hybridization between S_{NP} and R on the surface of GUVs. Confocal laser scanning microscopy images of the binding of ^{Cy3}R_{GUV} and ^{Cy5}S_{NP} on the surface of giant unilamellar vesicles (GUVs) composed of POPC lipids. Three channels are shown: the brightfield in the top row confirms the presence of GUV's, the second row shows the green channel exciting the Cy3 fluorophore on R, the third row shows the red channel exciting the Cy5 on S_{NP}. (a) Fluorescent green halos around the GUVs confirm insertion and tethering of ^{Cy3}R into the bilayer. (b) Both green and red fluorescent halos around the GUVs reaffirm (A) as well as successful hybridisation of Cy5S_{NP} with ^{Cy3}RGUV. (c) The absence of any fluorescent halos confirms that without the presence of Cy3R on the membrane surface Cy5S_{NP} does not interact with the GUVs.

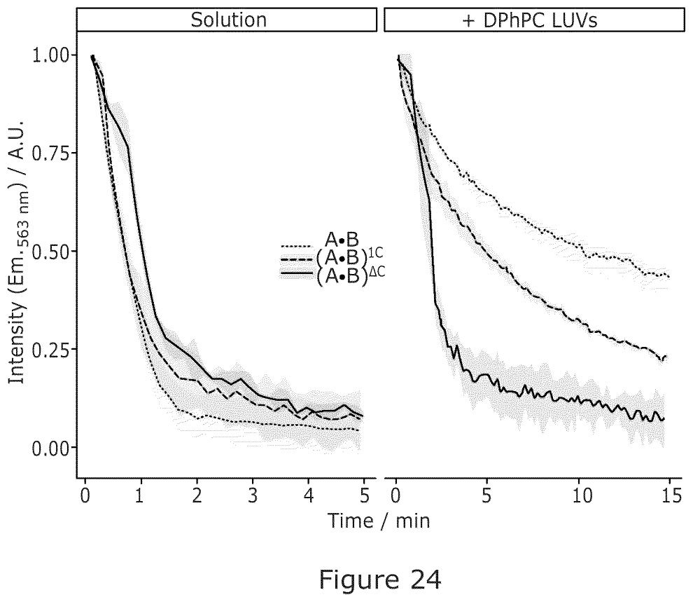

FIG. 24 shows the results of a digestion assay to confirm nanopore insertion. In solution, both (A·B)^{1C} and A·B are fully and rapidly digested by the BAL-31 DNA digestion enzyme. When (A·B)^{1C} and A·B are first incubated with LUVs composed of DPhPC, digestion is slowed in both instances likely because of steric hindrance from the vesicles. Digestion is significantly inhibited by the insertion of A·B into the lipid bilayer making the strand nicks of the DNA inaccessible. In the case of (A·B)^{1C}, access to the nicks is reduced by the presence of the membrane, but only on the side with the cholesterol lipid anchor.

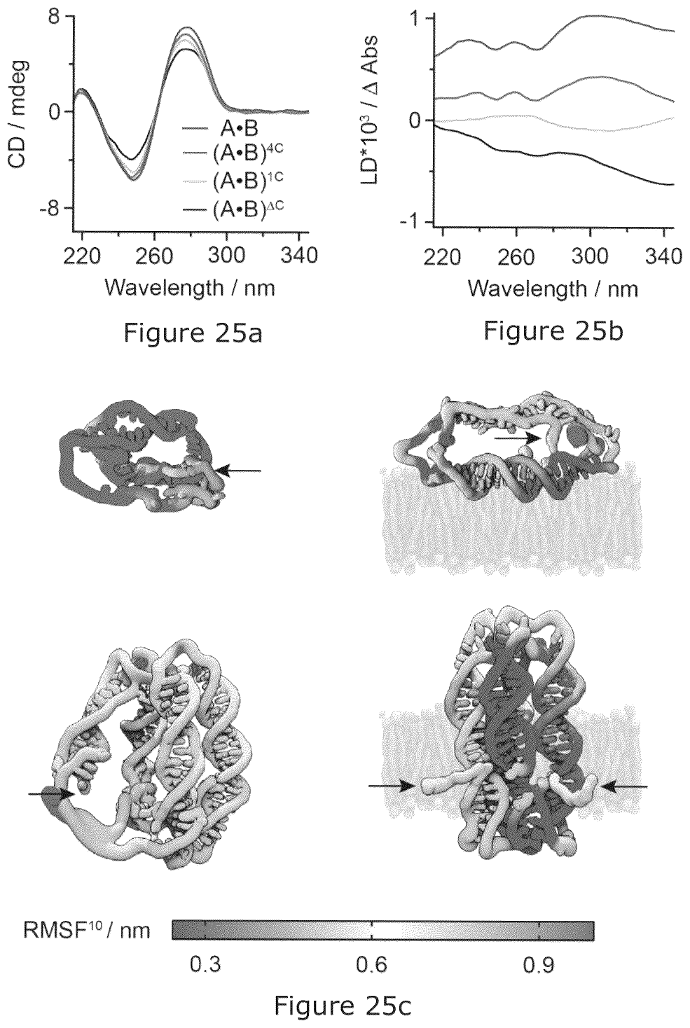

FIGS. 25a-25c show stability and dynamics of membrane tethered and membrane spanning DNA nanostructures. (a) Representative CD spectra of A·B (dark grey) and controls (A·B)^{4C} (lighter grey), (A·B)^{1C} (grey), and (A·B)^{ΔC} (black). (b) Representative LD spectra of A·B (dark grey), (A·B)^{4C} (mid grey), (A·B)^{1C} (light grey), and (A·B)^{ΔC} (black) in the presence of SUVs composed of POPC. (c) Representative snapshots of the DNA nanostructures from four simulated trajectories. Component A in solution (top left) and tethered to a membrane (top right), and A·B in solution (bottom left) and spanning a membrane (bottom right). The simulated POPC bilayer is shown in grey, and solvent atoms are omitted for clarity. DNA nanostructures are shaded by the per-residue RMSF¹⁰ to indicate regions of structural flexibility (left to right on scale). The location of the hydrophobic membrane anchors is indicated with black arrows.

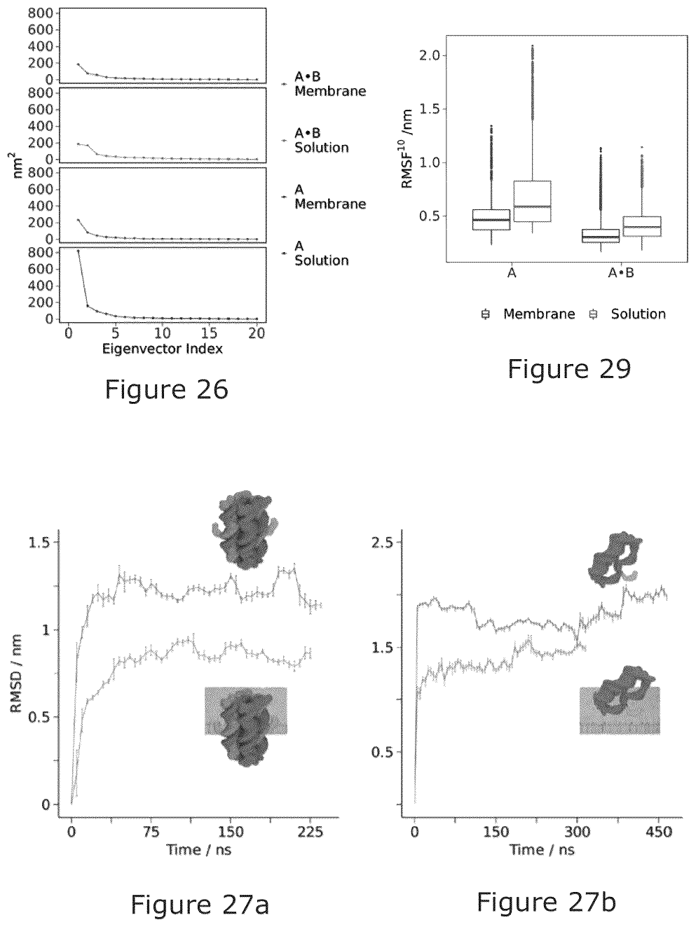

FIG. 26 shows Eigenvector index showing slowest modes of simulated trajectories. Eigenvector index was generated through Principle Component Analysis using gmx_covar and gmx_anaeig, demonstrating the first ten slowest modes describe over 90% of the structural dynamics of the DNA heavy atoms.

FIGS. 27a-27b show time dependent Root Mean Square Deviation (RMSD) of DNA heavy atoms for the 4 simulated trajectories. (a) RMSD for the nanostructure A·B in a 1 M KCl solution (top) or embedded within a POPC bilayer (bottom). (b) RMSD for component A in a 1 M KCl solution (top) or tethered to a POPC bilayer (bottom). The RMSD was calculated using initial idealised structures composed of rigid B-form DNA helices. The data is plotted as a 10 ns running average. Error bars represent the standard deviation

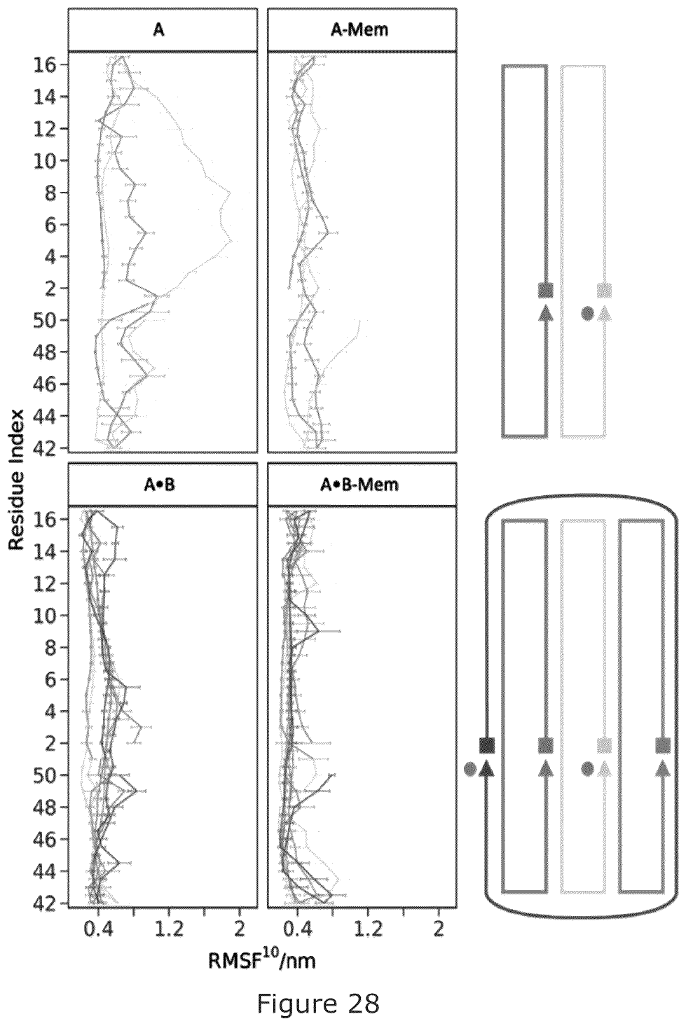

FIG. 28 shows Per-residue Root Mean Square Fluctuations¹⁰ (RMSF¹⁰) of the DNA nanostructures from the four simulated trajectories. The residue index on the Y axis has been ordered to match that of the associated strand maps map (Top: A; Bottom: A·B). Squares represent the 5′ and triangles the 3′ end of each DNA strand. The grey circle indicates a cholesterol anchor modification attached to the 3′ end of a DNA strand via tri(ethylene) glycol (TEG) linker.

FIG. 29 is a Boxplot of RMSF¹⁰ showing the values for Component A and nanostructure A·B in solution and interacting with membranes. The boxplot shows the RMSF¹⁰ values shown as the arithmetic average of the DNA heavy atoms of each of the four simulated trajectories

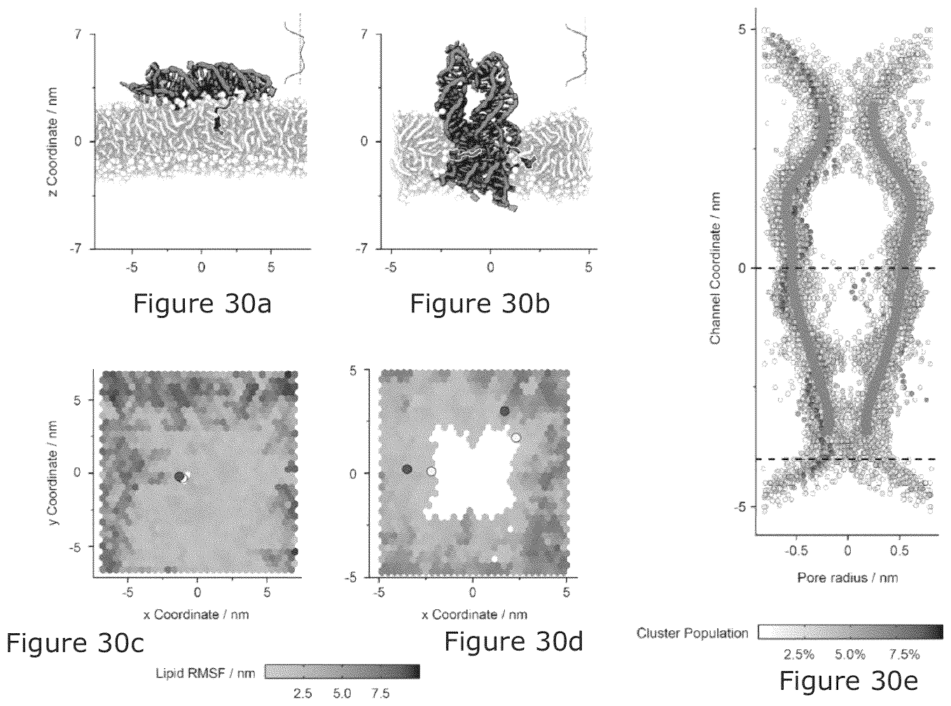

FIG. 30a-30e shows membrane anchoring of component A and nanopore A·B affects lipid bilayer as analyzed with molecular dynamics simulations. (a, b) Representative snapshots of the equilibrated regions of trajectories of (a) membrane-tethered component A and (b) membrane-spanning A·B. The insets show the initial configurations. The DNA is colored purple, and the TEG-cholesterol linkers are gold. The POPC bilayer is colored white (headgroups) and grey (lipid tails). Solvent is omitted for clarity. The average bilayer density from A and A·B trajectories are plotted to the upper-right of each snapshot, with a dashed line indicating the core density of the membrane lacking a transmembrane nanopore (0.425 g/mol/A³). (c,d) Top-down views corresponding to panels (a) and (b) respectively, with DNA omitted for clarity. Each hexagon is colored by the per-molecule-lipid RMSF (black=low, white=high). The locations of the cholesterol lipid anchors are indicated at the start (orange) and end (yellow) of the trajectory. (e) Simulated channel lumen mapping using HOLE analysis on a series of clustered snapshots of the A·B channel in a POPC bilayer in 1 M KCl. The proportion of the trajectory represented by each cluster is indicated by the transparency of the points, and a trend line has been plotted to estimate the average channel shape. The two black dashed lines represent the approximate positions of the bilayer headgroups compared to the nanopore channel coordinate in the most populated cluster.

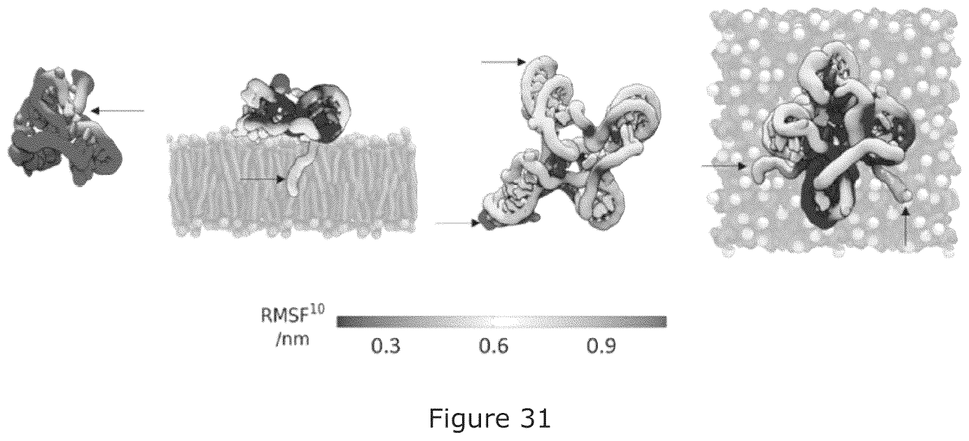

FIG. 31 shows top-down images showing representative structures for the four simulated trajectories, coloured by per-residue RMSF¹⁰. From left to right; the A subunit in solution, the A subunit tethered to a POPC bilayer, the A·B nanopore in solution and the A·B nanopore embedded in a POPC bilayer. For clarity the solvent has been omitted and the bilayer has been made transparent. The location of the hydrophobic cholesterol anchors has been indicated with a black arrow.

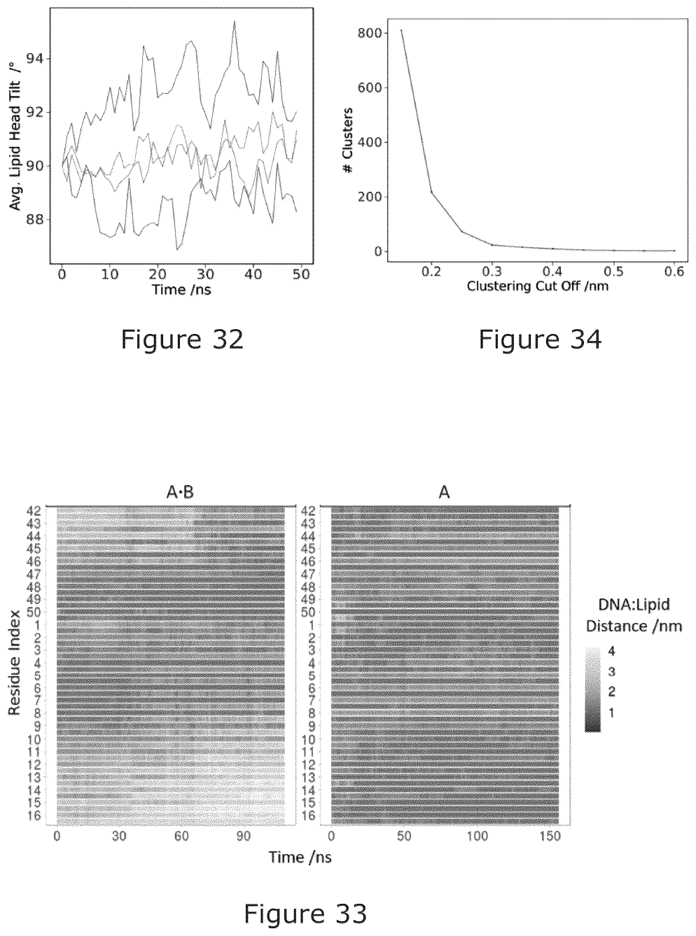

FIG. 32 shows a graph indicating the average angle of the lipid head groups relative to the bilayer normal. Insertion of the A·B nanostructure results in rapid formation of a toroidal lipid. The headgroups from the trajectory with the membrane spanning A·B DNA nanopore are shown in red and the headgroups from the trajectory with the membrane tethered Component A are shown in blue. The lipids were split by leaflet, and lipids further than 4 nm from the DNA structures were discarded, and the angle calculated with gmx_gangle and time averaged to give an indication of the direction of motion. The initial region of the NvT equilibration trajectory had to be used to capture this motion, as it mostly occurred within the first 20 ns of the simulation.

FIG. 33 shows the output of the evolution of the simulated per residue distance between the lipid bilayer and DNA nanostructure, A·B, and component A. Calculated using gmx_mindist over the initial NvT equilibration trajectories to demonstrate the DNA nanopore induced lipid remodelling.

FIG. 34 shows the analysis of different clustering cut-off values for the clustered lumen analysis of the membrane embedded DNA nanostructure pore, A·B. Clustering was performed using the gromos method (see examples) on the NpT region of the trajectory, after discarding the initial 10% of the trajectory. The final clustering cut off used was 0.25 nm, selected to ensure there were no more than 50 clusters, and >10 contained 90% of the trajectory and fewer than 3 clusters containing a single species.

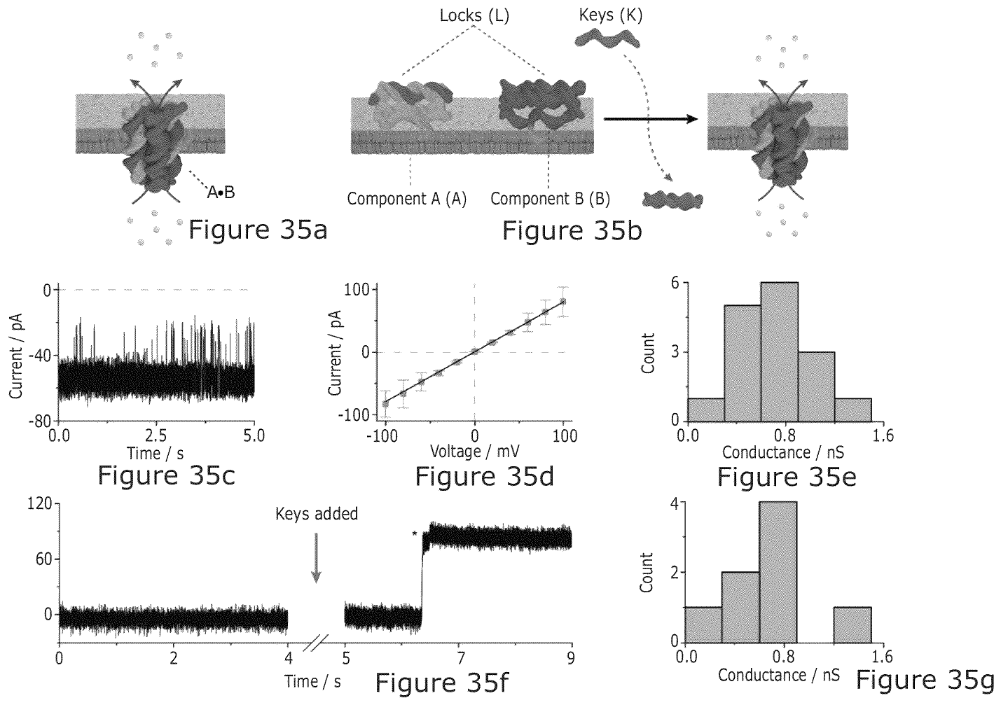

FIGS. 35a-35g show the characterization of the conductance properties of the nanostructure pore A·B. (a) Schematic illustration of a pre-assembled A·B inserted into a planar lipid bilayer. (b) Schematic illustration of triggered assembly at the membrane interface, induced by addition of key strands. (c) Representative ionic current traces from a single A·B nanopore inserted in a planar lipid bilayer composed of DPhPC lipids in 1 M KCl, 10 mM HEPES, pH 7.4 at a membrane potential of −50 mV. (d) Current-voltage relationship for voltages ranging from −100 mV to +100 mV at 20 mV steps, showing ohmic properties of the A·B channel and displaying averages±SEM from 16 independent pore insertions. (e) Histogram of channel conductance obtained from 16 independent single-channel recordings at membrane potentials ranging from +20 mV to +50 mV. (f) Representative ionic current trace showing how addition of the key strands to membranes containing AL^{A} and BL^{B} results in the formation of nanopore A·B at +90 mV. (g) Histogram of channel conductance obtained from 8 independent single-channel recordings of trigger assembled nanopores at membrane potentials of 20 mV.

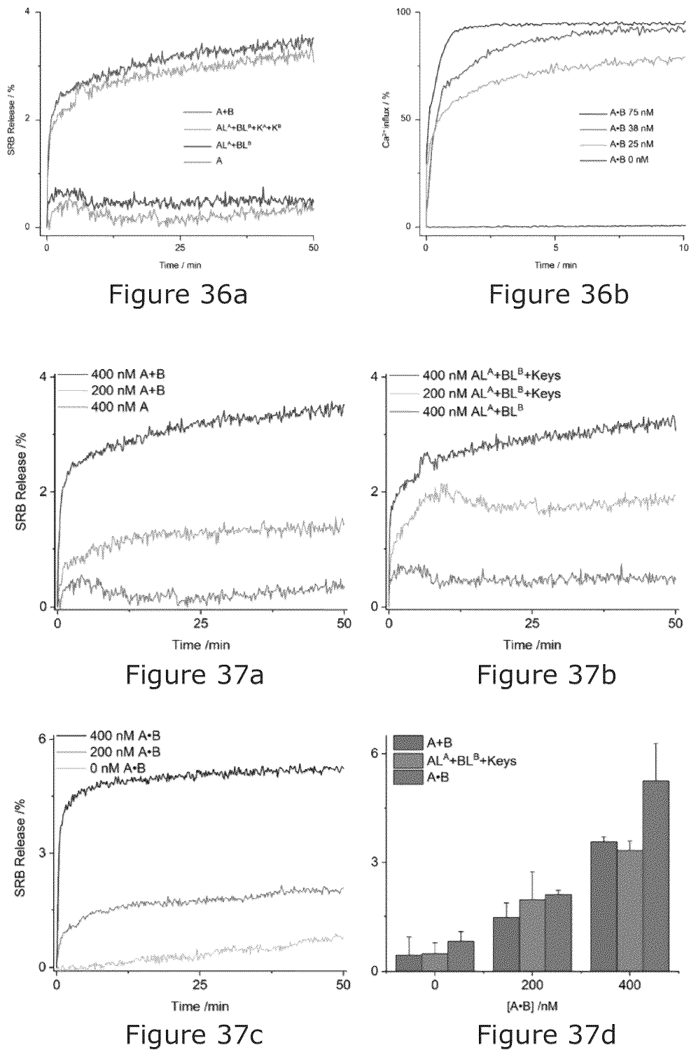

FIGS. 36a-36b show the graphs indicating that the transport-active A·B channels are formed by assembly from inactive components. (a) Kinetic fluorescence traces for sulforhodamine B (SRB) efflux mediated by direct assembly of A and B, and triggered assembly of A·B from inactive subunits AL^{A} and BL^{B}. The fluorescence signal originates from SRB that is contact-quenched at high concentrations within DPhPC SUVs but regains its emission when released at low concentrations into the ambient. A signal of 100% is obtained by rupturing the SUVs by addition of detergent. Each trace is the average of three independent repeats obtained at 400 nM. (b) Kinetic fluorescence traces on Ca²⁺ influx across membrane-inserted (A·B)^{4C} into POPC SUVs containing the Ca²⁺ sensitive dye Fura-2. The number of repeats for traces at 75 nM, 38 nM, 25 nM, and 0 nM are 3, 2, 1 and 3, respectively. Maximum influx was recorded following addition of a detergent to rupture the vesicles.

FIGS. 37a-37d show graphs of A·B concentration dependent SRB release from SUVs. (a) Concentration dependence when A·B was formed from unlocked components A+B (b), from locked components ALA+BLB upon addition of keys, (c) and pre-folded A·B. (d) Bar chart of net fluorescence increase summarizing the results from (a)-(c). 0 nM A·B represents the controls: 400 nM A only, 400 nM (ALA+BLB) and buffer. The data represent averages and standard deviations from three independent repeats.

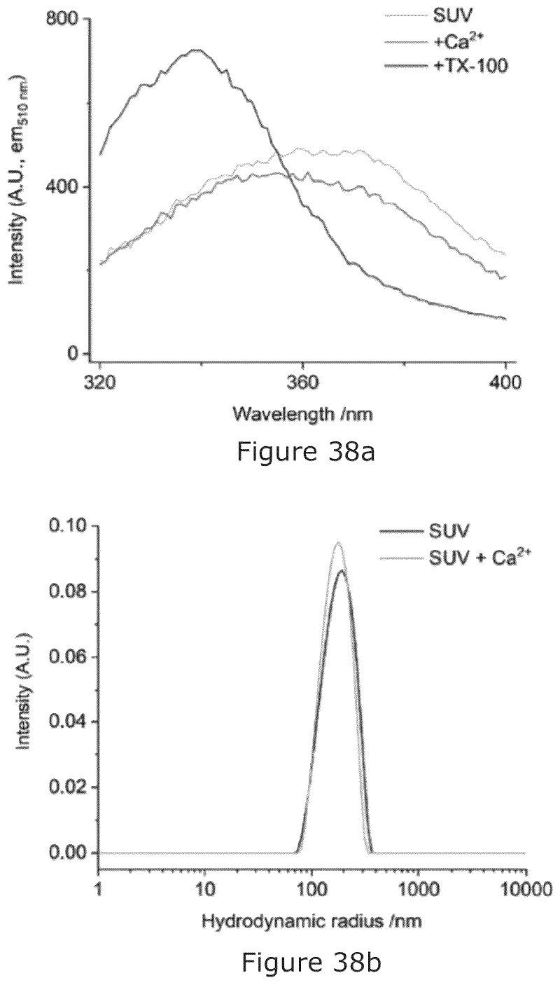

FIGS. 38a-38b show graphs indicating characterization of Fura-2-encapsulated vesicles for a Ca²⁺ influx assay. At low Ca²⁺ concentrations Fura-2 excites at 380 nm. As the concentration of Ca²⁺ increases, the excitation maximum shifts to 340 nm. The emission wavelength remains constant at 510 nm. Ca²⁺ binding can thus be assessed by monitoring the ratio of the excitation intensity at 340/380 nm. (a) Fluorescence scan of purified SUVs with encapsulated Fura-2 (100 μM) demonstrating the absence of Ca²⁺ in the vesicles and a low 340/380 ratio (0.95). Addition of 250 μM CaCl₂ results in a mild increase of the 340/380 ratio (1.13) consistent with some unpurified dye and confirms vesicles impermeability to both Fura-2 and Ca²⁺. Upon addition of a detergent (Triton X-100), the vesicles are lysed removing the barrier between Fura-2 and Ca²⁺ and resulting in maximum binding indicated by a significant increase in the 340/380 ratio (5.62). (B) Dynamic light scattering analysis of vesicles formed from POPC with encapsulated Fura-2 (100 μM) in the absence and presence of Ca²⁺. In the absence of Ca²⁺, SUVs had an average diameter of 186 nm, while in the presence of Ca²⁺, SUVs had an average diameter of 178 nm consistent with Ca²⁺ induced membrane compression.

### DETAILED DESCRIPTION OF THE INVENTION

Prior to setting forth the invention, a number of definitions are provided that will assist in the understanding of the invention.

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN:1934-3647); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, pages 2-662 (2011); Synthetic Biology, Part B, Computer Aided Design and DNA Assembly, Methods in Enzymology, Edited by Christopher Voigt, Volume 498, Pages 2-500 (2011. Each of these general texts is herein incorporated by reference.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘modular’ as used herein refers to the use of one or more units, or components, to design or construct a whole or part of a larger complex nanostructure. In the context of the present invention it refers to the use of individual component modules, sub-units or building blocks to construct a nanostructure, suitably a nanostructure configured to penetrate or traverse a semifluid or lipid bilayer membrane. The modules may be each the same or the modules may be different. To form the nanostructure, the individual modules are constructed so as to include assembly interface regions that facilitate assembly into a larger nanostructure complex via complementary base pairing at the assembly interfaces.

The modular design of a nanostructure may comprise a frame or framework of modules, and additional, typically smaller, sub-modules that connect, or support the frame, acting as struts or bracing members.

The modular components or sub-modules are comprised of nucleic acids, typically DNA, RNA and synthetic nucleic acids or analogues thereof (e.g. LNA or PNA). Each individual unit may be assembled by DNA/RNA origami techniques described elsewhere herein using suitably selected scaffold and staple strands in order to create a higher order structure—e.g., a secondary structure having defined geometric parameters. Such secondary structure may include the formation of A-, B- or Z-form double helices (duplex), triplex, quadruplex, hairpin loops, and trefoil structures as well as combinations of such structures.

The term ‘nucleic acid’ as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, and are typically manufactured synthetically, but may also be isolated from natural sources. Nucleic acids may further include modified DNA or RNA, for example DNA or RNA that has been methylated or that has been subject to chemical modification, for example 5′-capping with 7-methylguanosine or analogues thereof, 3′-processing such as cleavage and polyadenylation, and splicing, or labelling with fluorophores or other compounds. Nucleic acids may also include synthetic nucleic acids (XNA) or nucleic acid analogues, such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Hence, where the terms ‘DNA’ and ‘RNA’ are used herein it should be understood that these terms are not limited to only include naturally occurring nucleotides. Sizes of nucleic acids, also referred to herein as ‘polynucleotides’ are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 100 nucleotides in length are typically called ‘oligonucleotides’.

As used herein, the terms ‘3′’ (‘3 prime’) and ‘5′’ (‘5 prime’) take their usual meanings in the art, i.e. to distinguish the ends of polynucleotides. A polynucleotide has a 5′ and a 3′ end and polynucleotide sequences are conventionally written in a 5′ to 3′ direction. The term ‘complements of a polynucleotide molecule’ denotes a polynucleotide molecule having a complementary base sequence and reverse orientation as compared to a reference sequence.

The term ‘duplex’ is used herein refers to double-stranded nucleic acid hybridised molecules, such as DNA (dsDNA), meaning that the nucleotides of two complimentary DNA sequences have bonded together and then coiled to form a double helix (assuming A-, B- or Z-form), or also single-stranded RNA (ssRNA) that has annealed to a complimentary DNA sequence to generate an RNA-DNA hybrid (RDH) duplex. An RDH nanostructure may comprise a single RNA scaffold sequence with multiple shorter hybridised DNA sequences (e.g. DNA oligonucleotides) acting as staples forming a series of RDH duplexes along the length of the RNA scaffold thereby defining higher order structures.

According to the present invention, homology to the nucleic acid sequences described herein is not limited simply to 100%, 99%, 98%, 97%, 95% or even 90% sequence identity. Many nucleic acid sequences can demonstrate biochemical equivalence to each other despite having apparently low sequence identity. In the present invention homologous nucleic acid sequences are considered to be those that will hybridise to each other under conditions of low stringency (Sambrook J. et al, Molecular Cloning: a Laboratory Manual, Cold Spring Harbor Press, Cold Spring Harbor, NY). However, it may be desired in some cases to distinguish between two sequences which can hybridise to each other but contain some mismatches—an “inexact match”, “imperfect match”, or “inexact complementarity”- and two sequences which can hybridise to each other with no mismatches—an “exact match”, “perfect match”, or “exact complementarity”. Further, possible degrees of mismatch are considered.

As used herein, the term ‘nanostructure’ refers to a geometrically predefined or ‘predesigned’ two or three dimensional molecular structure typically comprised from a biopolymer, suitably a naturally or non-naturally occurring nucleic acid, which structure has at least one dimension or an aspect of its geometry that is within the nanoscale (i.e. 10⁻⁹ metres). Nanoscale structures suitably have dimensions or geometry of less than around 100 nm, typically less than around 50 nm, and most suitably around 20 nm. Nanoscale structures suitably possess dimensions or geometry greater than around 0.1 nm, typically greater than around 1 nm, and optionally greater than around 2 nm. Assembly of nucleic acid nanostructures may occur spontaneously in solution, such as by heating and cooling a mixture of DNA strands of preselected sequences, or may require presence of additional co-factors including, but not limited to, nucleic acid scaffolds, nucleic acid aptamers, nucleic acid staples, co-enzymes, and molecular chaperones. Where desired nanostructures result from one or more predesigned spontaneously self-folding nucleic acid molecules, such as DNA or RNA, this is typically referred to as nucleic acid ‘origami’. Rational design and folding of DNA to create two dimensional or three-dimensional nanoscale structures and shapes is known in the art (e.g. Rothemund (2006) Nature 440, 297-302). The term ‘geometrically predefined’ is used to mean that the geometry of the nanostructure is predefined such that upon assembly the nanostructure conforms to the desired shape and configuration intended by the designer. By way of example, selection of the scaffold and staple sequences is such that the rational design of the nanostructure is assured repeatedly upon completion of hybridisation.

In the classical scaffold-and-staple approach, one or more long biogenic scaffold strand component(s) is folded into a defined nucleic acid nanostructure optionally with a staple component consisting of a number of shorter synthetic staple oligonucleotides. Classical DNA nanostructures are formed of bundles of parallel aligned DNA duplexes that are arranged into bundles or barrel shapes that can enclose a central pore channel and puncture a semifluid membrane. Suitably, certain scaffold structures may be based off all or a part of an M13 or phiX174 sequences, with which a plurality of smaller staple and linker sequences configured to achieve the desired three-dimensional nanostructural geometry. In embodiments of the present invention alternative scaffolds may be utilised and may comprise artificial, or non-naturally occurring, sequences that are designed specifically for the task of nanostructural modular assembly. Typically, such sequences will be non-repetitive and with base selection that is optimised to facilitate nucleic acid hybridisation between component modules under conditions that favour nanostructure assembly.

The nucleic acid sequences that form the nanostructures will typically be manufactured synthetically, although they may also be obtained by conventional recombinant nucleic acid techniques. DNA constructs comprising the required sequences may be comprised within vectors grown within a microbial host organism (such as E. coli). This would allow for large quantities of DNA or RNA to be prepared within a bioreactor and then harvested using conventional techniques. The vectors may be isolated, purified to remove extraneous material, with the desired DNA sequences excised by restriction endonucleases and isolated, such as by using chromatographic or electrophoretic separation.

As used herein the term ‘hydrophobic’ refers to a molecule having apolar character including organic molecules and polymers. Examples are saturated or unsaturated hydrocarbons. The molecule may have amphipathic properties.

As used herein, the term ‘hydrophobically-modified’ relates to the modification (joining, bonding or otherwise linking) of a polynucleotide strand with one or more hydrophobic moieties. A ‘hydrophobic moiety’ as defined herein is a hydrophobic organic molecule and may be synonymous with the term ‘lipophilic’ indicating the molecule has an affinity for lipids and particularly the lipid core of a membrane bilayer. The hydrophobic moiety may be any moiety comprising non-polar or low polarity aliphatic, aliphatic-aromatic or aromatic chains. Suitably, the hydrophobic moieties utilised in the present invention encompass molecules such as long chain carbocyclic molecules, polymers, block co-polymers, and lipids. The term ‘lipids’ as defined herein relates to fatty acids and their derivatives (including tri-, di-, monoglycerides, and phospholipids), as well as sterol-containing metabolites such as cholesterol. The hydrophobic moieties comprised within the embodiments of the present invention are capable of forming non-covalent attractive interactions with amphipathic semifluid membranes or phospholipid bilayers, such as the lipid-based membranes of cells and act as membrane anchors for the nanostructure. According to certain embodiments of the present invention suitable hydrophobic moieties, such as lipid molecules, possessing membrane anchoring properties may include sterols (including cholesterol, derivatives of cholesterol, phytosterol, ergosterol and bile acid), alkylated phenols (including methylated phenols and tocopherols), flavones (including flavanone containing compounds such as 6-hydroxyflavone), saturated and unsaturated fatty acids (including derivatives such as lauric, oleic, linoleic and palmitic acids), and synthetic lipid molecules (including dodecyl-beta-D-glucoside). The anchors for the polymer membrane may be the same as for lipid bilayers or they may be different. The specific hydrophobic moiety anchor may be selected based on the binding performance of the membrane chosen.

In embodiments of the invention the disclosed nanostructures may comprise one or more hydrophobic or lipophilic anchors that act to attach or connect or anchor the hydrophilic nucleic acid nanostructure to a generally hydrophobic membrane such as a semifluid or lipid bilayer of a vesicle. The lipid anchors are attached to the nanostructure or comprised within modules that form part of overall the nanostructure. Suitably attachment is via oligonucleotides that carry the lipid anchor, suitably cholesterol, at the 5′ or 3′ terminus. Polynucleotides or oligonucleotides may be functionalized using a modified phosphoramidite in the strand synthesis reaction, which is easily compatible for the addition of reactive groups, such as cholesterol and lipids, or attachment groups including thiol and biotin. Enzymic modification using a terminal transferase can also be used to incorporate an oligonucleotide, which incorporates a modification such as an anchor, to the 3′ of a single stranded nucleic acid (e.g. ssDNA). These lipid modified anchor strands may hybridize via ‘adaptor’ oligonucleotides to corresponding sections of the nucleic acid sequence forming the scaffold section of the nanostructure. Alternatively, the lipid anchors are assembled with the nanostructure using lipid-modified oligonucleotides that contribute as either the scaffold or staple strands. A combination of approaches to anchoring using two or more membrane anchors may also be adopted wherein anchors are incorporated into one or all of a scaffold strand, a staple strand and an adaptor oligonucleotide. Cholesterol has been found to be a particularly suitable lipid for use as an anchor in the present invention (see FIG. 1). The use of other lipids as anchors is contemplated, although it may be expected that there is a particular preference for a particular lipid, and a given number of membrane anchors, for a given membrane chemistry.

In an alternative embodiment of the invention, the hydrophobic modification is comprised within one or more synthetic nucleic acids (XNAs) incorporated into the nanostructure structure itself.

A nanostructure according to an embodiment of the present invention is able to associate with, or bind to, a cell a membrane or to a microsomal or exosomal structure within the body of a subject. In an embodiment of the invention, the nanostructure associates with a membrane via insertion of a least one associated hydrophobic anchor moiety into the membrane bilayer. According to this embodiment of the invention a majority of the nanostructure is localised to an outer surface of the membrane but does not penetrate or puncture the membrane in the manner of a membrane-spanning nanopore.

Suitably the nanostructures of the invention comprise one or more polynucleotide strands that provide a functional scaffold component, wherein the polynucleotide strands comprised within the scaffold component include a polynucleotide backbone; and a plurality of polynucleotide strands that provide a plurality of functional staple components. The scaffold strand(s) cooperate with and hybridise to themselves or the plurality of staple polynucleotide strands—e.g. via appropriate Watson-Crick base pairing hybridisation—in order to form a three-dimensional configuration of the nanostructure, which is termed the secondary structure.

A nanostructure according to an embodiment of the invention may comprise a nucleic acid nanostructure such as a nanobarrel. Hence, the nucleic acid may be formed into a bundle, or a series of modules comprised of bundles, that cooperate to define the desired geometry of nanostructure. The geometry may comprise a combination of secondary structural motifs and more open unstructured regions. The geometry may change from pre-assembly to post-assembly.

The nanostructures of all configurations of the present invention may be configures via the ‘scaffold-and-staple’ approach. In this important route to nucleic acid nanostructures, DNA or RNA is utilized as a building material in order to make nanoscale three dimensional shapes. The arrangement of complex nanostructures from a plurality of un-hybridized linear molecules is typically referred to as ‘nucleic acid origami’. The nucleic acid origami process generally involves the folding of the one or more elongate, ‘scaffold’ strands into a particular shape via self-hybridisation and/or by using a plurality of rationally designed ‘staple’ oligonucleotide strands. The scaffold strand can have any sufficiently non-repetitive sequence. The sequences of the staple strands are designed such that they include sequences that hybridize to particular defined portions, or regions, of the scaffold strands and, in doing so, these two components cooperatively force the scaffold strands to assume a particular structural configuration. Staple strands are typically made from DNA but may also comprise RNA, or other synthetic nucleic acids as described above. Methods useful in the making of DNA origami structures can be found, for example, in Rothemund, P. W., Nature 440:297-302 (2006); Douglas et al, Nature 459:414-418 (2009); Dietz et al, Science 325:725-730 (2009); and U.S. Pat. App. Pub. Nos. 2007/0117109, 2008/0287668, 2010/0069621 and 2010/0216978, each of which is incorporated by reference in its entirety. Staple sequence design can be facilitated using, for example, CaDNAno software, available at http://www.cadnano.org or the DAEDALUS online platform, available at http://daedalus-dna-origami.org.

In embodiments of the invention the staple and/or scaffold components further comprise a plurality of hydrophobic/lipophilic membrane anchor molecules that are attached thereto. The hydrophobic anchors (or portions of the sequence) facilitate association the nanostructure components with a semifluid membrane pre- or post-assembly. During or after the process of assembly into a complete nanostructure the presence of the membrane anchor molecules facilitates the penetration of the membrane by the nanostructure and helps stabilise the nanostructure within the membrane. Typically, the membrane anchor molecule may be tethered to the nanostructure components at a position that is substantially equatorial—e.g. in a location that is along the midline of the nanostructure when embedded within the membrane—such that the anchor molecule extends radially outwardly from the nanostructure into the surrounding semifluid membrane. Each individual component module may comprise at least one, two, three or more membrane anchors depending upon the size and stability requirements of the module and ultimately the fully assembled membrane-spanning nanostructure.

According to one embodiment of the present invention a nucleic acid nanostructure is provided that comprises a plurality of component modules. Suitably, each module comprises a nucleic acid sequence (e.g. DNA) and at least one attached membrane anchor molecule. The nucleic acid sequence comprises at least a portion/region of double helix that serves to define a secondary structural element having a defined length as well as a level of structural rigidity. The secondary structure may comprise one or more nucleic acid duplex bundles. In a specific embodiment of the invention, the duplex bundles may be oriented 5′ to 3′ substantially perpendicularly to the planar axis of the semifluid membrane. In a further embodiment of the invention, the duplex bundles may be oriented 5′ to 3′ substantially co-axially to the planar axis of the semifluid membrane. In some nanostructures there may be a combination of modules comprising both orientations in combination depending on the requirements of the resultant nanostructure.

The nucleic acid sequences also comprise at least a portion of single stranded sequence that defines an assembly interface. The assembly interface may be exposed, which facilitates spontaneous assembly of the nanostructure in solution or when complementary component modules are co-located on a membrane surface. Upon assembly the nanostructure shifts conformation such that it becomes able to penetrate and span the semifluid membrane.

In a further embodiment, a plurality of single stranded nucleic acid lock sequences that are capable of hybridising with the single stranded sequence of the assembly interface are provided. The lock sequences act to inhibit spontaneous assembly of the nanostructure either in solution or when the component modules are applied to the membrane surface. The lock sequences may be displaced or induced to disassociate from the assembly interface sequences upon addition of an external stimulus—e.g. via a trigger mechanism—which then permits spontaneous assembly of the nanostructure and consequent penetration of the membrane. Hence, this controlled initiation of a three-dimensional nanostructure from a plurality of dissociated component modules in response to a predefined stimulus is referred to as a‘triggered’ assembly The external stimulus may comprise a trigger molecule selected from one or more of the group consisting of: a nucleic acid sequence; an aptamer; a small molecule; an antibody or an antibody fragment; an antibody mimetic; a peptide; a polypeptide; a polysaccharide; and an oligosaccharide. In a specific embodiment the external stimulus comprises a nucleic acid sequence that hybridises to all or a part of a lock sequence, or to a portion of the lock sequence sufficient to induce dissociation from the assembly interface sequence of a component module.

In embodiments of the invention the lock sequence may hybridise imperfectly to the assembly interface sequence, for example, with one or more base pair mismatches. In an alternative or additional embodiment, the lock sequence may comprise one or more moieties that are attached and cause a level of steric hindrance. In both such embodiments the energetics may favour disassociation of the lock sequence in the presence of an external stimulus, such as a perfectly matched trigger nucleic acid sequence. In further embodiments of the invention the lock sequence may comprise or be linked to an aptamer that binds to a trigger molecule as described above. Upon binding of the trigger molecule the aptamer may undergo a conformational change that results in disassociation of the lock sequence from the assembly interface sequence.

Hence, in embodiments the nanostructures of the present invention are formed or constructed from a plurality of discrete component modules that cooperate spontaneously or in response to an external stimulus to form a complete membrane spanning complex. In embodiments, the nanostructure may be formed of an arrangement of modules that forms a basic frame or framework. In embodiments, the modules of the frame are supported by additional, typically smaller, sub-modules that connect and support the structure of the frame. In embodiments of the present invention the modules and sub-modules may comprise a plurality of substantially similar scaffold and staple nucleic acid structures that are assembled in the same way, and which associate to form a repeating structural motif.

While any arrangement of the modules is contemplated, suitably, following assembly the modules may be arranged to form a range of membrane spanning nanostructures having a polygonal cross-section. The component modules are arranged such that they sit side by side thereby defining the geometric configuration of the overall nanostructure. The modules may have tuneable side length (a side length in this context being defined as the longest dimension of the module), which when chosen with an appropriate final overall shape, allows for different sized and/or shaped nanostructures to be prepared. For example, the nanostructures defined by the assembly of component modules may include a range of three-dimensional geometric shapes, suitably selected from regular or irregular polyhedrons, with a cross section defining annular or solid shapes such as a circle, an oval, a triangle, a quadrilateral (e.g. a square, a rectangle or a trapezoid), a pentagon, a hexagon, a heptagon, an octagon and so on. As disclosed herein, in specific embodiments, the nanostructures resemble nanobarrels or helical bundles. However, it will be appreciated that the geometry of the nanostructure may be selected to accommodate a range of factors. These factors may be dependent upon inherent properties of the membrane thickness, such as the length required to span the membrane; or may be defined by stability or functionality factors such as the desired diameter of an enclosed lumen in the case of a membrane-spanning nanopore.

Typically, a side length or maximum dimension of the nanostructures, or modules that are comprised within the nanostructures, is in the order of between 5 nm and 50 nm. Suitably, the side length of the modules may be at least 2 nm, 3 nm, 4 nm, 5 nm, 6 nm, 7 nm, 8 nm, 9 nm or 10 nm, Suitably the side length of the modules may be at most around 100 nm, 50 nm, 40 nm, 30 nm, 20 nm and 10 nm. The sizing of component sub-modules may be determined by the number of modules required for the complete assembly, or the relative spacing between the modules which is turn is determined by the shape of the nanostructure and the size and number of modules employed. Suitably, the side length of a component module may be at least 0.5 nm, 1 nm, 1.5 nm, 2 nm, 2.5 nm, 3 nm, 3.5 nm, 4 nm or 5 nm, Suitably the side length of a component module may be at most 20 nm, 10 nm, 9 nm, 8 nm, 7.5 nm, 7 nm, 6 nm or 5 nm.

In accordance with an embodiment of the present application, the nanostructure defines at least one lumen that extends along a central axis of the nanostructure thereby defining at least a first and a second opening. Hence, the assembled nanostructure may be a membrane-spanning nanopore. The first and second openings may be referred to as apertures (e.g. first and second apertures) and permit fluid communication through the lumen—or central channel—of the pore. Suitably the first aperture is located on the cis side of the nanopore and the second aperture on the trans side. When the nanopore is embedded within a membrane the fluid communication through the pore permits a measurable flow of electrically charged ions to pass through the pore from cis to trans or vice versa—i.e. a measurable electrical current can pass across the membrane via the lumen of the nanopore. It will be appreciated that in measurable current will not pass across the membrane until the nanostructure has assembled and penetrated the membrane. In this way, sensing or drug delivery functionality is controlled by the ability of the nanostructure to assemble from its component modules. In embodiments of the invention where assembly is controlled by an external stimulus, the sensing or drug delivery capability is consequently subject to the presence of the stimulus.

The three-dimensional configuration of an assembled nanopore of the present invention defines at least one channel, suitably a single channel that spans the membrane, the channel having a lumen that has a minimum internal width of at least about 0.2 nm, suitably 0.5 nm, optionally 0.75 nm. Fully assembled nanopores of the present invention typically may have a single channel located at least substantially centrally in the pore structure when viewed perpendicular to the plane of the membrane in which the pore is intended to reside. The channel defines the lumen that passes through the nanopore which is perpendicular to the planar axis defined by the membrane. The minimum opening, or aperture, of the channel in this cross-section (e.g. the minimum constriction) is suitable to facilitate a close-fitting interaction with a folded protein or other analyte in solution, or to allow passage of a small molecule there-through. Typically, the minimum opening of the lumen is at least 1 nm, 2 nm, 3 nm, 5 nm, 7.5 nm, 8 nm, 8.5 nm, 9 nm, 9.5 nm, 10 nm, 11 nm, 12 nm, 13 nm, 14 nm, or 15 nm or more. Suitably the lumen is between around 1 nm and around 20 nm in width. Suitably, the maximum opening of the channel (i.e. minimum constriction) is at most 200 nm, 150 nm, 100 nm, 75 nm, 50 nm, 40 nm, 30 nm, 20 nm, 18 nm, 15 nm, 12 nm, or 10 nm.

### Use of the Nanostructures in Nanopore Sensing Applications

In one embodiment of the invention, a signal readout is generated via measurement of an ionic electrical current that flows through a fully assembled nanopore from the first side to a second side of the membrane (e.g. cis to trans, or trans to cis), by way of a gradient of soluble ions present in the solution. The flow of this electrical current is measurable over a given period of time. This permits the nanostructures of the invention to find utility in nanopore sensing devices such as those used in in ‘chip-based’ nanopore sequencing and analytical sensor applications. For example, devices such as the MinION® system sold by Oxford Nanopore Technologies®; the GS FLX+® and the GS Junior® System sold by Roche®; the HiSeq®, Genome Analyzer IIx®, MiSeq® and the HiScanSQ® systems sold by Illumina®; the Ion PGM® System and the Ion Proton System® sold by Life Technologies; the CEQ® system sold by Beckman Coulter®; and the PacBio RS® and the SMRT® system sold by Pacific Biosciences®. It will also be appreciated that alternative readouts may exist for identifying when an analyte molecule is located optimally within the pore lumen. For example, alternative detection modalities based on field-effect transistors (FET), quantum tunnelling and optical methods such as fluorescence and plasmonic sensing may be utilised. For instance, a combination of a solid-state FET nanopore with an adjacent nanoribbon, nanotube, or nanowire, allows for sensing analyte molecules that interact with the lumen of the pore thereby disrupting the local electrical ionic current passing through the pore. In an alternative embodiment, transverse electrical measurements across the membrane of voltage, current or impedance may be made in order to generate a detectable signal readout in the presence of analyte. It will be appreciated that whichever readout technology is adopted, the trigger-based assembly mechanism prevents leakage of current in the absence of the stimulus and may, therefore, serve to reduce background noise and/or spurious results.

Non-naturally occurring amphiphiles and amphiphiles which form a semifluid amphiphilic membrane layer are known in the art and include, for example, block copolymers (Gonzalez-Perez et al., Langmuir, 2009, 25, 10447-10450). The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane may be chosen one of the membranes disclosed in PCT/GB2013/052767, hereby incorporated by reference in its entirety. The amphiphilic molecules may be chemically-modified or functionalised to facilitate coupling an insertion of the nanostructure. The membrane can comprise both a lipid and an amphiphilic polymer such as disclosed in PCT/US2016/040665.

Polymer-based semifluid membranes may be formed of any suitable material. Typically, synthetic membranes are composed of amphiphilic synthetic block copolymers. Examples of hydrophilic block copolymers are poly(ethylene glycol) (PEG/PEO) or poly(2-methyloxazoline), while examples of hydrophobic blocks are polydimethylsiloxane (PDMS), poly(caprolactone (PCL), poly(lactide) (PLA), or poly(methyl methacrylate) (PMMA). In embodiments, the polymer membrane used may be formed from the amphiphilic block copolymer poly 2-(methacryloyloxy)ethyl phosphorylcholine-b-disisopropylamino) ethyl methacrylate (PMPC-b-PDPA). The membrane is typically planar, although in certain embodiments it may be curved or shaped. Amphiphilic membrane layers may also be supported. Suitably the membrane is a lipid bilayer or monolayer. Methods for forming lipid bilayers are known in the art such as disclosed in International Application Number PCT/GB2008/000563. Lipid bilayers are commonly formed by the method of Montal and Mueller (Proc. Natl. Acad. Sci. USA., 1972; 69: 3561-3566).

In specific embodiments, one or more of the component modules of the nanostructure may further comprise one or more binding moieties which may be comprised of an affinity binding component or a molecule that is able to bind to an affinity binding molecule, such as an antigen. A binding moiety is suitably located on a component module proximate to the lumen aperture or within the lumen of a nanopore when fully assembled. This facilitates binding to an analyte within the vicinity of the first aperture. Alternatively, the affinity binding moiety may respond to an external stimulus and initiate the described process of nanostructure assembly. In an alternative embodiment the binding moiety is tethered to the fully assembled nanopore at a location that allows the binding moiety to project fully or partially outside of the lumen. Upon binding of an analyte, the flow of an electrical current through the lumen of the nanostructure, is obstructed causing a blockade of all or a major portion of current passing through the lumen. The level of signal readout—such as current blockade—can be measured in order to identify that the analyte has been detected. The one or more binding moieties may be attached to the nanostructure prior to assembly or after assembly and insertion into the membrane via a covalent or non-covalent linkage, such as via avidin-biotin or His-tag type interaction.

The affinity binding moiety may comprise a polynucleotide or a polypeptide that is capable of binding to an analyte, that may serve as an external stimulus, present in a solution that surrounds the component modules (prior to assembly) or the membrane-embedded fully assembled nanopore. Where the binding molecule comprises a polypeptide or polynucleotide that is tethered to a component module or to the nanopore, either within the lumen, an aperture or proximate to the cis or trans side of the nanopore, it may be selected from one of the group consisting of:
I. an enzyme—including a polymerase, a helicase, a gyrase, and a telomerase, as well as nucleic acid binding sub domains or derivatives thereof
II. synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. synthetic or naturally derived affinity binding nucleic acids and nucleic acid analogues, including oligonucleotide probes, aptamers and ribozymes;
V. naturally occurring or synthetic small molecules, including drugs, fluorophores, metabolites and chemokines;
VI. an antigen or antigenic fragment; and
VII. signalling molecules and/or polypeptide receptors thereof, including binding domains of receptors, and receptor complexes.

Alternatively, the affinity binding molecule may comprise a small molecule, a lipid group, a polysaccharide group, a polymer or any other molecule naturally-occurring or synthetic molecule that is capable of effecting a specific affinity binding interaction with an analyte in solution.

The assembled nanopore structures of the present invention are suited to use in sensor applications that allow for the detection of a diverse range of potential analytes that may exist in a solution that is under test. Exemplary analytes may include:
peptides/polypeptides/proteins—folded, partially/completely unfolded;
enzymes;
protein/nucleic acid constructs;
molecules defined by size;
macromolecules within specified size ranges, for example, in ranges selected from: 1-10 kD, 1-50 kD, 1-100 kD, 10-50 kD, 10-100 kD, 20-50 kD, and 20-100 kD;
multi-protein complexes;
antigens or their antibodies;
glycoproteins;
carbohydrates;
biopolymers;
toxins;
metabolites and by products thereof;
cytokines;
nucleic acids.

The nanopore structures of the present invention may be incorporated within a plurality of improved devices and sensors. Such devices and sensors are useful in applications requiring to sensing and characterization of a variety of materials and analytes. By way of non-limiting example, particularly useful applications, including genome sequencing, protein sequencing, other biomolecular sequencing, and detection of ions, molecules, chemicals, small molecules, biomolecules, metal atoms, contaminants, polymers, nanoparticles etc. Such detecting and characterizing can, in turn, be used to diagnose diseases, in drug development, to identify contamination or adulteration or food or water supplies, and in quality control and standardization.

According to exemplary embodiments of the present invention, a sensor device typically comprises a substrate that includes a membrane into which one or more assembled nanopores are embedded, or onto which one or more component modules are anchored prior to exposure to an assembly trigger. The substrate is placed to facilitate contact with a fluid (optionally an electrolytic solution) which comprises an analyte, or an assembly trigger molecule. At least one, and optionally a plurality of, device(s) are positioned relative to the substrate, wherein a given device generates a signal (e.g. mechanical, electrical, and/or optical) in response to detecting binding to and/or passage through the nanopore(s) of one or one or more analytes. The plurality of devices can be greater than 2 and as many as 100, or as many as 20, or as many as 10, or between 2 and 8 devices. Each device may be selected from one or more of the group consisting of: a field effect sensor; a plasmonic sensor; a laser based sensor; an interferometric sensor; a wave-guide sensor; a cantilever sensor; an acoustic sensor; a quartz crystal microbalance (QCM) sensor; an ultrasonic sensor; a mechanical sensor; a thermal sensor; an optical dye based sensor; a fluorometric sensor; a calorimetric sensor; a luminometric sensor; a graphene sensor; a quantum dot sensor; a quantum-well sensor; a photoelectric sensor; a 2D material sensor; a nanotube or nanowire sensor; an enzymatic sensor; an electrochemical sensor, including a FET or BioFET sensor; a potentiometric sensor; a conductometric sensor; a capacitive sensors; and an electron-spin sensor. The devices may cooperate in the form of arrays allowing for multiplexed testing of multiple analytes. The sensor devices may further comprise special purpose hardware and systems (e.g., circuitry, processors, memory, GUIs etc.) that perform the specified functions or acts, or combinations of special purpose hardware and computer instructions, in order to render a functioning sensor device capable of providing a meaningful readout to a user.

Hence, according to embodiments of the invention suitably configured nanopore devices may enable a variety of different types of sensor measurements to be made. Typically, electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with or based upon electrical measurements (Soni G V et al., Rev Sci Instrum. 2010 January; 81(1):014301), for instance, via conversion of ionic current into a fluorescent signal from an indicator dye (e.g. Fluo-8) arising from Ca2+ flux through a nanopore (Huang et al. Nat Nanotechnol. 2015 November; 10(11): 986-991). As mentioned previously, the measurement may be a transmembrane current or voltage measurement such as measurement of ionic current flowing through the nanopore. Alternatively, the signal may be obtained from measurement of a change in transverse membrane current, voltage and/or impedance value over time.

### Use of Nanostructures in Drug Delivery

The nanostructures described in embodiments of the present invention may also find utility as membrane-embedded synthetic molecular gates that open in response to a specific stimulus. The stimulus may allow for a defined molecular recognition system to be incorporated into a vesicle/liposomal based drug or imaging substance delivery platform. Hence, the synthetic molecular gates are comprised of fully assembled nanopores that regulate flux of bioactive or otherwise detectable substances across a vesicular/liposomal membrane. Bioactive substances, referred to as ‘pharmaceutical agents’, may include small molecules, such as drug compounds; or biologicals such as coding or non-coding mRNA, siRNA, aptamers, monoclonal or polyclonal antibodies or fragments and mimetics thereof. Controllable delivery of therapeutic drugs to cellular environments in response to a biologically relevant exogenous trigger is described in Lanphere et al. (2021) Ang. Chemie, 60(4): 1903-1908. Imaging substances may include substances useful in nuclear medicine and/or clinical diagnosis, such as radionuclides of technetium, thallium, gallium, iodine, and/or xenon. By way of example, bioimaging substances may be used for more basic research including in vivo imaging with fluorescent probe-labeled biomolecules. Different imaging materials are suitably used for visualizing target molecules, cells, tissues, and organs in different modalities depending upon diagnostic or clinical need. The imaging substance may be comprised within a companion diagnostic composition that provides information that is essential for the safe and effective use of a corresponding therapeutic product. For example, the imaging substance may comprise a radioisotope or fluorescently labelled antibody, antibody fragment of mimetic thereof.

When administered to a subject, a therapeutic component is suitably administered as part of the in vivo delivery composition and may further comprise a pharmaceutically acceptable vehicle in order to create a pharmaceutical composition. Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. Pharmaceutical compositions, if desired, can also contain minor amounts of wetting or emulsifying agents, or buffering agents.

Medicaments and pharmaceutical compositions of embodiments of the invention can take the form of liquids, solutions, suspensions, gels, modified-release formulations (such as slow or sustained-release), emulsions, capsules (for example, capsules containing liquids or gels), liposomes, microparticles, nanoparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

For any compound or composition described herein, the therapeutically effective amount can be initially determined from in vitro cell culture assays. Target concentrations will be those concentrations of active component(s) that are capable of achieving the methods described herein, as measured using the methods described herein or known in the art.

As is well known in the art, therapeutically effective amounts for use in human subjects can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

It is contemplated that embodiments of the invention may include compositions formulated for use in medicine. As such, the composition of the invention may be suspended in a biocompatible solution to form a composition that can be targeted to a location on a cell, within a tissue or within the body of a patient or animal (i.e. the composition can be used in vitro, ex vivo or in vivo). Suitably, the biocompatible solution may be phosphate buffered saline or any other pharmaceutically acceptable carrier solution. One or more additional pharmaceutically acceptable carriers (such as diluents, adjuvants, excipients or vehicles) may be combined with the composition of the invention in a pharmaceutical composition. Suitable pharmaceutical carriers are described in ‘Remington's Pharmaceutical Sciences’ by E. W. Martin. Pharmaceutical formulations and compositions of the invention are formulated to conform to regulatory standards and can be administered orally, intravenously, topically, intratumorally, or subcutaneously, or via other standard routes. Administration can be systemic or local or intranasal or intrathecal. In particular, particular compositions according to the invention can be administered intravenously, intralesionally, intratumorally, subcutaneously, intra-muscularly, intranasally, intrathecally, intra-arterially and/or through inhalation.

The invention is further illustrated by the following non-limiting examples.

### Example

This example demonstrates the formation of a membrane-spanning nanopore using an exogenous trigger/stimulus to initiate its assembly in situ from two independent component modules.

Controlled assembly of non-spanning subunits into a barrel-like nanopore is functionally complex and offers a clear turn-on signal that avoids the leakage seen with lidded nanopores. Novel trigger-assembled pores also add scientific breadth by integrating several fundamental processes that underpin their formation: (i) molecular recognition—between the trigger and the pore subunits to activate them for interaction, (ii) conformational changes—of pore subunits at the membrane to prime them for interaction, and (iii) molecular assembly—of the activated subunits to form a unitary nanostructure that defines a membrane spanning pore channel. By integrating the three processes, synthetic triggerable nucleic acid nanostructural pores are hereby provided that mimic the actions of many dedicated naturally occurring membrane proteins that have evolved to carry out these tasks.

DNA nanotechnology provides a versatile route for biomimetic design. DNA nanotechnology offers high structural precision, tuneability and dynamic-nanomechanical control along with chemical modifications for expanding functional interactions with biomolecules, including bilayers. Building on these strengths, rational design with DNA has yielded barrel-like membrane pores with tune-able lumen diameters. The structural dynamics of DNA nanopores and their molecular interaction with the bilayer membranes has been studied with molecular dynamics (MD) simulations complementing computational studies on biological pores. Design with DNA has also led to pores that unblock the channel lumen in response to stimuli, such as oligonucleotides, proteins, or temperature, and controllably capped nanotubes. However, DNA membrane nanopores with an in situ controlled assembly have not been built so far. These DNA structures can be used for sensing, cell biological research or drug delivery as described previously.

Here the inventors enlist DNA nanotechnology to construct a functionally advanced membrane pore that assembles from two unique subunits after triggered activation (FIG. 1). The controlled formation integrates the processes of molecular recognition between the triggers and the inactive subunits, the repositioning of the activated subunits within the membrane, and their assembly into a functional channel (FIG. 1). The affinity and kinetics of pore formation are determined to reveal any influence of the lipid bilayer on the pathway. Furthermore, molecular dynamics simulations explore the structural dynamic changes associated with repositioning of the membrane-bound DNA components during assembly. Finally, fluorophore and ion flux are assayed to probe the efficiency of transport across the assembled DNA channel.

### Results

DNA nanopore design. A DNA nanopore capable of assembling from constituent components at the membrane interface (FIG. 1) and in solution was designed. The nucleic acid sequences are set out in Table 6 and various assemblies described in Table 7. The nanopore, denoted A·B, consists of a bundle of four DNA helices and assembles from components A and B. Each component is made from two DNA strands that form a central duplex, a ssDNA loop and two ssDNA arms (FIG. 1, FIG. 2 and FIG. 3). The pore assembles from the components by hybridization between the ssDNA arms of one and the loop of the other component, thereby forming two additional duplexes. The resulting pore's nominal dimensions are 7.5 nm in height and 5.1 nm in outer width. The inner channel lumen is up to 0.8 nm wide.

To control pore formation, the components can be rendered assembly-inactive with two lock strands, L^{A} and L^{B}. In the inactive components AL^{A} and BL^{B}, the lock strands sequester the ssDNA arms in a second duplex (FIG. 1, FIG. 3), thus rendering them incapable of binding the other component. However, the lock strands feature a 10-nucleotide overhang which allows for their selective removal by addition of a key strand via toehold-mediated-strand displacement (FIG. 1, FIG. 3)⁵⁴ Addition of key strands, K^{A} and K^{B}, sequesters the lock strands and reveals an assembly interface that restores the ability of components A and B to cooperate to form a pore (FIG. 1). To facilitate membrane binding, each component carries a lipophilic cholesterol anchor. After pore assembly, the cholesterol anchors are symmetrically positioned on opposite sides of the pore (FIG. 1, FIG. 3).

A·B assembles directly or via triggered activation. Direct pore assembly was first assessed in solution. Using gel electrophoresis as a read-out, isolated components A and B appeared as fast migrating single bands (FIG. 4a, lanes 1-2), implying a homogenous population. By comparison, mixing of A and B (FIG. 4a, lane 3) resulted in a significant band shift, indicating the formation of the larger, assembled 4-duplex pore A·B (FIG. 4a, lane 3). The lack of other significant bands suggests that pore assembly was quantitative.

Pore formation also proceeded via triggered assembly in solution. The two components with locks, AL^{A} and BL^{B} (FIG. 4a, lanes 4-5), migrated higher than components A and B without locks, as expected for constructs of greater masses. In further agreement, when mixed, the assembly-locked components AL^{A} and BL^{B} migrated without additional bands that would indicate interaction (FIG. 4a, lane 6). Addition of stoichiometric amounts of the keys K^{A} and K^{B}, however, resulted in the complete removal of AL^{A} and BL^{B} bands and the concomitant formation of a higher migrating A·B band (FIG. 4a, lane 7) that matched the one from direct pore formation. The assembly by-products, duplexes L^{A}K^{A} and L^{B}KB, migrate to the bottom of the gel (FIG. 4a, lane 7). Pore assembly was further demonstrated with cholesterol-free components A^{ΔC}, B^{ΔC} and the corresponding locked components A^{ΔC}L^{A} and B^{ΔC}L^{B} (FIG. 5, FIG. 6) The data show that pore formation in solution is not influenced by the absence or presence of the cholesterol tags. The equivalence of the pore formed by direct assembly compared to an annealed control was also confirmed with UV-thermal melting analysis. The melting profiles of A^{ΔC}+B^{ΔC} and (A·B)^{ΔC} were very close (FIG. 7a) resulting in identical melting temperatures (Tm) of 63.1±0.1° C. and 63.1±0.2° C., respectively (n=3, FIG. 7b).

Following successful confirmation of triggered pore assembly, the effect of varying the key concentration on assembly was investigated. Addition of the key, K^{A}, to the corresponding assembly-locked component, A^{ΔC}L^{A}, led to the expected component unlocking with no other interactions even at a 10-fold excess of the key (FIG. 6a). When the noncomplimentary key, K^{B}, was added to A^{ΔC}L^{A}, the formation of the ternary complex A^{ΔC}L^{A}K^{B} (FIG. 6b) was observed. In this complex, K^{B} is bound to component A^{ΔC} given their partial sequence complementarities. This is an implicit consequence of designing a trigger-assembled duplex bundle. The same is true for the binding of K^{A} and B^{ΔC}L^{B}. In a further experiment, both keys were added at increasing concentrations to both assembly-locked components (FIG. 6c). As previously demonstrated, a 1:1 ratio of key:assembly-locked component led to quantitative pore formation. However, when an excess of both keys was added, pore assembly was inhibited. The side products were the ternary complexes A^{ΔC}L^{A}K^{B} and B^{ΔC}L^{B}K^{A} where the bound mismatched key blocks assembly to the pore. The experiment also led to the formation of a new complex which ran slightly above the band for the folded pore (A·B)^{ΔC} (FIG. 6c). The new complex is an incompletely folded, open barrel-like structure in which bound keys prevent assembly into the 4-duplex pore (FIG. 6c). This effect can be addressed by making accommodations for the key design or nature of the trigger stimulus in subsequent pore arrangements.

Affinity and kinetics of pore assembly in solution. After confirming pore assembly, the equilibrium dissociation constant, K_{d}, and the kinetic rate constant, kₒₙ was determined. An electrophoretic mobility shift assay (EMSA) was used to derive K_{d}. Visually tracking pore formation over a range of ratios of A^{ΔC}:B^{ΔC} resulted in the expected binding profile (FIG. 8a,b) and 1:1 binding stoichiometry. Plotting the gel band intensities (FIG. 8a, b) yielded a Langmuir-derived Kd of 154±23 nM (n=3).

As the EMSA derived Kd may be influenced by the limited sensitivity of ethidium bromide staining, the more sensitive detection method of Förster resonance energy transfer (FRET) was used. For this analysis, components A^{ΔC} and B^{ΔC} were labeled with FRET donor dye Cy3 and acceptor dye Cy5, respectively. Component mixing led to the expected FRET signal when the dyes are proximal upon pore formation. In particular, Cy3 emission at 563 nm was reduced and the Cy5 emission at 670 nm was increased (FIG. 14b). Quantitative analysis of the fluorescence signals after titrating ^{Cy5}B^{ΔC} over constant ^{Cy3}A^{ΔC} using the same ratios as used for EMSA but at an 2.5-fold lower concentration yielded a K_{d} of 53.1±5.4 nM (n=3, FIG. 9a,b).

The FRET derived K_{d} was corroborated using dual-color fluorescence cross-correlation spectroscopy (FCCS). Using a 10-fold lower concentration range than in FRET, FCCS measurements led to a Kd of 62.2±12.5 nM (n=3, FIG. 10a) which is within error of the Kd obtained from FRET (Table 1). Dropping the concentrations of the labeled components further 5-fold did not lead to saturation in binding (FIG. 10a). In further analysis, FCCS did not indicate any significant difference between the binding strength of either component; when the concentration of B^{ΔC} was held constant while A^{ΔC} was varied, the K_{d} was 71.7±7.7 nM (n=2)(FIG. 10b) which is within error of the other K_{d}. The K_{d} is more than order of magnitude higher than the affinity obtained for other DNA duplexes of comparable length. This likely reflects the molecular difference between sterically restrained duplex formation within the DNA nanopore, and binding of two conformationally unlocked single stranded DNA strands.

TABLE 1
Summary of equilibrium and kinetic data for the assembly of nanopore
A · B from components A and B in solution (A^{ΔC} + B^{ΔC}), at
the membrane interface (A-SUV + B^{ΔC}) and on the membrane
surface (A-SUV + B) using Förster resonance energy transfer (FRET).

| Parameter | Condition | Avg. ± Std. Dev.^{a} |
|---|---|---|
| K_{d} (M) | A^{ΔC} vs B^{ΔC} | 5.3 ± 0.5 × 10⁻⁸ |
| | A-SUV vs B^{ΔC} | 5.6 ± 0.2 × 10⁻⁸ |
| | A-SUV vs B | 5.6 ± 0.6 × 10⁻⁸ |
| kₒₙ (M⁻¹s⁻¹) | A^{ΔC} + B^{ΔC} | 11.9 ± 2.8 × 10³ |
| | A-SUV + B^{ΔC} | 5.9 ± 0.6 × 10³ |
| | A-SUV + B | ^{b} 1.9 ± 0.3 × 10³ |
| k_{off} (s⁻¹) | A^{ΔC} + B^{ΔC} | 6.3 ± 1.9 × 10⁻⁴ |
| | A-SUV + B^{ΔC} | 3.1 ± 0.4 × 10⁻⁴ |
| | A-SUV + B | ^{b} 1.1 ± 0.2 × 10⁻⁴ |

After determining K_{d}, the kinetic rate constant, kₒₙ of pore assembly was measured. Using EMSA, we examined whether the kinetics of triggered assembly are different to direct assembly. The kₒₙ obtained for direct (4.5±0.4×10³ M⁻¹ s⁻¹, n=3, FIG. 4b, FIG. 11a,b) and triggered assembly (4.7±0.5×10³ M⁻¹s⁻¹, n=3. FIG. 4b, insert; FIG. 11c,d) were within error. This indicates that the triggering mechanism is not rate-limiting. ^{a} Averages and standard deviations were obtained from at least three independent repeats. ^{b} kₒₙ derived from kinetic trace with initial fluorescence drop removed

We confirmed the EMSA-derived kinetic data with a FRET-based continuous kinetic assay. The time-dependence of the FRET signal after component mixing yielded a kₒₙ of 11.9±2.8×10³ M⁻¹ s⁻¹ (n=3, Table 1, FIG. 12), which is close to the EMSA-derived value. The rate constants at around 10³ M⁻¹ s⁻¹ are two-three orders of magnitude slower than typical DNA duplex hybridization. The slower kinetics are likely a result of slow nucleation due to the presence of additional sequences of the non-target arms and loops and slow zippering due to significant secondary structure resulting from arm and loop interactions.

Pore assembly at the membrane interface. After characterizing pore formation in solution, we investigated pore assembly at the membrane interface. We first incubated cholesterol-tagged ^{Cy3}A with giant unilamellar vesicles (GUVs), then added the non-cholesterol modified ^{Cy5}B^{ΔC} and detected the lipid-anchor-mediated membrane tethering using confocal microscopy. Overlapping Cy3 and Cy5 fluorescent halos around the GUVs suggest that the two components assembled into pore (A·B)^{1C} at the membrane interface (FIG. 14a and FIG. 13).

To obtain quantitative information on pore assembly at the membrane, we used a FRET assay using small unilamellar vesicles (SUVs). As a baseline, we first added ^{Cy3}A to SUVs and acquired a Cy3 emission spectrum (FIG. 3b, pink, FIG. 9). Adding the second component, ^{Cy5}B, to membrane-anchored ^{Cy3}A in a 1:1 ratio resulted in a FRET-induced decrease in Cy3 and an increase in Cy5 emissions, respectively, implying pore assembly (FIG. 14b, purple). The emission spectrum overlapped with a control trace for an A·B pore that was pre-annealed before addition to SUVs (FIG. 14b, dark purple, FIG. 9). The spectral equivalence indicates quantitative assembly on the membrane surface. Part of the change in fluorescence is likely caused by the proximity of the fluorophores when ^{Cy3}A and ^{Cy5}B are tethered to the membrane, but not assembled. This contribution can be seen by a weak FRET change upon adding assembly inactive ^{Cy3}ALA and ^{Cy5}BL^{B} in a 1:1 ratio to the SUVs, which resulted in a FRET change equivalent to 32.0±3.9% assembly (n=3, FIG. 15a). By contrast, A+B resulted in a signal change of 96.3±6.6% relative to the pre-annealed control confirming pore assembly (FIG. 14c, FIG. 15b).

To additionally probe for insertion of A·B pores into SUV membranes, the melting profiles were analyzed, as bilayer insertion is known to confer increased stability to DNA pores and a higher Tₘ.^{32, 65} The Tₘ values for A·B assembled on the membrane and pre-annealed prior to SUVs incubation were 3° C. higher than for the non-SUV sample (FIG. 6) implying membrane insertion.

Affinity and kinetics of pore assembly at membranes. The equilibrium dissociation constant, K_{d}, for pore formation at the bilayer interface was obtained by adding component ^{Cy5}B to ^{Cy3}A-anchored SUVs and measuring the change in FRET. Plotting the normalized FRET signal as a function of ^{Cy5}B concentration (FIG. 9e,f) led to a K_{d} of 55.8±6.2 nM (n=3, Table 1). This is within error of the K_{d} values for pore formation in solution as well as for assembly of cholesterol-free ^{Cy5}B^{ΔC} with SUV-bound ^{Cy3}A (Table 1, FIG. 9a-d). In agreement, yields for pore assembly obtained from FRET efficiency (E) calculations revealed that pore assembly occurs in high yield across all three conditions (Table 2). A similar equivalence of affinity values in pore formation was obtained using EMSA analysis (K_{d}=1.64±0.14×10-7 M, n=3 FIG. 8c,d).

TABLE 2
Summarized FRET efficiency (E) data for pore assembly derived from the
FRET pore assembly binding titrations and pore assembly kinetics. The
pre-annealed control for each of the three conditions was derived from
the FRET binding data and was used as a benchmark. Averages and standard
deviations were calculated from at least three independent repeats.

| | Pre-folded | | In situ yield: | | In situ yield: |
|---|---|---|---|---|---|
| Condition | control (E) | Binding (E) | binding (%) | Kinetics (E) | kinetics (%) |
| A^{ΔC} + B^{ΔC} | 0.27 ± 0.07 | 0.26 ± 0.07 | 96.3 ± 42.1 | 0.26 ± 0.02 | 95.6 ± 31.1 |
| A-SUV + B^{ΔC} | 0.28 ± 0.02 | 0.26 ± 0.03 | 94.8 ± 14.7 | 0.31 ± 0.05 | 113 ± 22.7 |
| A-SUV + B | 0.36 ± 0.03 | 0.34 ± 0.03 | 96.3 ± 13.4 | ^{a} 0.35 ± 0.02 | 98.7 ± 11.4 |
| | | | | ^{b} 0.31 ± 0.04 | 87.2 ± 15.4 |

^{a} derived from kinetic trace with initial drop included
^{b} derived from kinetic trace with initial drop removed

By contrast, kinetic FRET analysis of ^{Cy3}A and ^{Cy5}B assembly on the membrane (FIG. 12) revealed an association rate constant, kₒₙ, of 1.9±0.3×10³ M⁻¹ s⁻¹ (n=3), which is one order of magnitude slower than in solution (Table 1). Likely, pore formation on the membrane surface is sterically hindered by membrane anchoring. It is noted that the kₒₙ value is corrected for an initial sharp drop in Cy3 emission when ^{Cy5}B is mixed with vesicles carrying membrane-anchored ^{Cy3}A (FIG. 12a). It occurs most likely because the rapid binding of ^{Cy5}B to the membrane leads to close proximity to ^{Cy3}A and a weak FRET signal. As support of this interpretation, the extent of the rapid initial drop, obtained via normalized intensity (F/F₀), was 0.91±0.03 (FIG. 12a), which is close to the value of 0.92±0.04 (FIG. 15) for the previously discussed assembly-blocked AL^{A}+BL^{B}. The fast process of membrane binding occurs before the slower pore assembly.

The experimentally determined K_{d} and kₒₙ were used to calculate the dissociation rate constant, k_{off}, using equation (1) assuming a second-order system:

$${K}_{d} = \frac{{k}_{off}}{{k}_{on}} \qquad ( 1 )$$

The k_{off} for pore assembly at membranes is 1.94±0.53×10⁻⁴ s⁻¹, which is two to three orders of magnitude slower than typical values for simple DNA hybridization that range between 10⁻¹-10⁻³ s⁻¹. The lower k_{off} is plausible given the required multiple duplex dissociations to separate the pore into its two components. Other contributions come from the movement of the separated components against the lateral membrane pressure and repositioning of the separated pore components from a membrane spanning to tethering orientation. The quantitative kinetic analysis was complemented by visually tracking pore assembly on supported lipid bilayers using single molecule FRET (smFRET) and single particle tracking (FIG. 16). The analysis yielded a FRET efficiency of 0.39±0.3 very close to the values found for assembly on the vesicle membranes (Table 2).

Probing the influence of steric factors on hybridization. The kinetic analysis of A·B pore formation at membranes revealed that kₒₙ and k_{off} are strongly different to solution. The likely reason is that duplex formation and dissociation requires the DNA components to change their position from a membrane adhering to membrane spanning state, and back, respectively. This theory was corroborated with a model system where DNA hybridization is taking place outside the membrane and hence expected to be less influenced by steric factors. The model was based on DNA duplex hybridization of a 20-nt DNA strand, S, to a complementary strand, R (FIG. 17, FIG. 18, Tables 8 and 9). To probe for the influence of steric bulk, S is optionally carrying a six-duplex DNA barrel of 15.5×5.5 nm while R is optionally cholesterol-anchored to SUV membranes. Hybridization was assessed for all conditions using EMSA and FRET (FIG. 19, FIG. 20, FIG. 21, FIG. 22). In line with expectations, analysis of DNA hybridization by EMSA (FIG. 19 and FIG. 21) revealed that K_{d} is largely unaffected by the absence or presence of the nanobarrel (Table 3, FIG. 19, FIG. 21). Membrane anchoring led to weaker affinity, but not in the presence of the nanobarrel where the affinity was unaffected. Similarly, kₒₙ values were not largely influenced by bulk or membrane anchoring (Table 3, FIG. 22, FIG. 23). The values were also in line with literature studies on DNA duplex formation. This suggests that the slower kinetics for A·B pore assembly is to a large extent a consequence of the previously discussed slow nucleation and zippering steps. The duplex insertion into the bilayer also likely has an effect on the kinetics.

TABLE 3
Summary of thermodynamic and kinetic data obtained for the
four conditions of the model system. Assembly in solution for R vs S
and R and S_{NP} as well as at the membrane interface when R
is bound to SUVs composed of DOPC:DOPE in a 7:3 mole
ratio, R_{SUV} vs S; and R_{SUV} vs S_{NP}. Averages and standard
deviations were obtained from three independent repeats.

| Parameter | Condition | Avg. ± Std. Dev. |
|---|---|---|
| K_{d} | S vs R | 4.05 ± 0.36 × 10⁻⁸ |
| (M) | S_{NP} vs R | 8.01 ± 0.33 × 10⁻⁸ |
| | S vs R_{SUV} | 15.4 ± 2.36 × 10⁻⁸ |
| | S_{NP} vs R_{SUV} | 7.86 ± 1.91 × 10⁻⁸ |
| kₒₙ | S vs R | 10.7 ± 0.87 × 10⁵ |
| (M⁻¹s⁻¹) | S_{NP} vs R | 4.79 ± 0.24 × 10⁵ |
| | S vs R_{SUV} | 9.16 ± 0.89 × 10⁵ |
| | S_{NP} vs R_{SUV} | 3.88 ± 0.44 × 10⁵ |
| k_{off} | S vs R | 4.33 ± 0.53 × 10⁻² |
| (s⁻¹) | S_{NP} vs R | 3.84 ± 0.25 × 10⁻² |
| | S vs R_{SUV} | 14.1 ± 2.60 × 10⁻² |
| | S_{NP} vs R_{SUV} | 3.05 ± 0.82 × 10⁻² |

As a further insight from the DNA duplex model, the FRET-derived extent of assembly dropped by ˜40% at the membrane interface compared to solution (Table 4), consistent with previous reports. In contrast, not only were the yields of A·B pore assembly not significantly affected by the membrane, but all conditions are higher than the model system (Table 2). This high yield is attributed to the previously noted highly favorable pore formation (Tables 1 and 2).

TABLE 4
Summary of FRET efficiencies derived from the hybridization
kinetics between the four components of the model
system. Averages and standard deviations were calculated
from at least three independent repeats.

| | FRET Efficiency, E | Extent of reaction |
|---|---|---|
| Condition | Avg. ± Std. Dev. | (norm. % vs control) |
| Control | 0.80 | 100 |
| S vs R | 0.74 ± 0.01 | 92.6 ± 1.77 |
| S_{NP} vs R | 0.66 ± 0.05 | 82.8 ± 8.84 |
| S vs R_{SUV} | 0.37 ± 0.02 | 46.1 ± 3.54 |
| S_{NP} vs R_{SUV} | 0.45 ± 0.06 | 55.8 ± 10.6 |

Investigating the orientation of A·B at the membrane interface. We first probed the orientation of A·B relative to the bilayer membrane using a nuclease digestion assay (FIG. 24). In the assay, membrane-inserted DNA pores are partly protected from digestion by the nuclease compared to more sterically accessible DNA pores in solution. Indeed, solvated pores with zero, one and two cholesterols, (A·B)^{ΔC}, (A·B)^{1C} and A·B, respectively, were rapidly and completely digested (FIG. 24). By comparison, incubating pores with large unilamellar vesicles (LUVs) prior to nuclease incubation led to the anticipated a cholesterol-dependent reduction in both the rate and extent of digestion (FIG. 24). Non-cholesterol (A·B)^{ΔC} was unaffected as it does not bind to the sterically protecting membrane. In contrast, (A·B)^{1C} gained protection by LUVs (FIG. 24) as the single cholesterol is expected to tether the pore parallel to the membrane (FIG. 1, FIG. 3b) and render it less accessible to the nuclease. Finally, double cholesterol-tagged A·B experienced the biggest nuclease protection (FIG. 24) in line for a pore that is expected to span the lipid bilayer (FIG. 1, FIG. 3b). These measurements to do not inform about which percentage of (A·B) pores span the membrane.

To corroborate the cholesterol-dependent orientation of our DNA nanopore, dichroism spectroscopy was used. Using circular dichroism (CD) spectroscopy the helical-structure of the nanopore was ascertained. The CD spectra of A·B constructs with either 4, 1 or no cholesterols in the absence of membranes exhibited the characteristic signature for the expected B-form DNA with a negative peak at 245 nm and a positive peak at 280 nm (FIG. 25a). The lack of competing peaks in the CD spectra suggests that all constructs form typical B-type helical structures.

Linear dichroism (LD) was then used to probe the orientation of the A·B pore relative to SUV membranes. A positive peak at 260 nm in the LD spectra indicated that A·B with two cholesterol anchors (FIG. 25b, dark purple) was oriented perpendicular to the bilayer⁷³⁻⁷⁴ indicative of a membrane-spanning orientation. In further agreement, pore variant (A·B)^{4C} with four cholesterols yielded a similar spectrum (FIG. 25b, upper lines). The spectrum of control pore (A·B)^{ΔC} without cholesterol featured a flattened spectral line (FIG. 25b, bottom line) as expected for a lack of membrane interaction. The broad negative LD peak for (A·B)^{1C} with one cholesterol (FIG. 25b, grey) suggests a dynamic orientation parallel to the bilayer. The noisy nature of the spectra is assumed to be in part due to the effects of light scattering from the SUVs with diameters of ˜180 nm.

Molecular dynamics (MD) simulations provide insight into the structural dynamics of pore components and the pore. The membrane dependent interactions of component A and assembled A·B were further investigated using atomistic molecular dynamics. Insight from experimental data was used to inform the initial configurations of the simulated trajectories. Structural dynamics were investigated using the per-residue root mean square fluctuation (RMSF¹⁰) (FIG. 26, FIG. 27, FIG. 28). The baseline analysis of component A in solution yielded a highly labile structure, with an average regional fluctuation of 0.72±0.38 nm (FIG. 25c, top-left; FIG. 28). Addition of a lipid bilayer membrane, however, stabilized component A to an average region fluctuation of 0.49±0.17 nm (FIG. 25c, top-right; FIG. 28). The stabilization is likely caused by cholesterol tag-mediated proximity of the DNA nanostructure to the membrane resulting in electrostatic interactions between the DNA duplex and lipid headgroups (FIG. 29).

In comparison to component A, pore A·B in solution was significantly more stable yet remained dynamic with an average regional RMSF of 0.42±0.14 nm (FIG. 25c, FIG. 27, FIG. 28). The simulations also indicate that the A·B structure is linked via terminal crossovers, and nicks in each duplex are a hinge-point (FIG. 25c, FIG. 2). The TEG-cholesterol groups were highly mobile, coiling inside the attached or neighboring duplex, consistent with previous reports. By contrast, membrane interaction stabilized the A·B pore resulting in a lowered regional averaged RMSF of 0.35±0.15 nm and a compact pore geometry (FIG. 25c, FIG. 28). As a likely reason for stabilization, the lipid bilayer reduces the structural flexibility of the pore's central lumen and strand breaks.

Increased stabilization was also found for a duplex (RMSF of 0.2-0.3 nm) positioned between two cholesterol-modified duplexes (RMSF of 0.3-04 nm) (FIG. 25c, FIG. 27). This stabilization is likely due to linker-cholesterol groups which are positioned close to the unmodified duplexes and thereby reduce the dynamics of the surrounding phospholipid molecules.

The membrane-induced changes from a globular structure to a compact pore were corroborated by comparison with the average intra-fluorophore distance of the Cy3-Cy5 on A·B (Table 5), which is a useful proxy for pore diameter. The Cy3-Cy5 distances were derived from the FRET efficiency as described.⁷⁸ The experimental data support the membrane-induced compression of the inserted pore as the intra-fluorophore distances for the non-membrane-spanning pore (A·B)^{ΔC} at 7.10±0.50 nm dropped slightly for an A·B pore within SUVs to 6.63±0.15 nm. In agreement, control pore (A·B)^{1C} in a membrane-tethered, but not compressing state, remained at the solution-phase distance of 7.05±0.14 nm.

TABLE 5
Calculated Cy3-Cy5 distances as an approximation for the average
diameter of the nanopore, A · B, in each condition. Distances
were calculated using the FRET efficiencies derived from the pre-folded
controls in Table S3 when R₀ = 5.6 or 6 nm for Cy3-Cy5.

Cy3-Cy5 Distance /nm

| Condition | R₀ = 5.6 | R₀ = 6.0 |
|---|---|---|
| (A · B)^{ΔC} | 6.63 ± 0.47 | 7.10 ± 0.50 |
| (A · B)^{1C}-SUV | 6.58 ± 0.13 | 7.05 ± 0.14 |
| A · B-SUV | 6.19 ± 0.14 | 6.63 ± 0.15 |

Membrane-pore-interactions alter the lipid bilayer structure and dynamics. MD simulations were also used to assess if and to what extent membrane-interacting component A and pore A·B altered the lipid bilayer structure and dynamics. Following the simulations, tethering of component A to the membrane resulted in minimal structural changes to the bilayer (FIG. 30a). Upon membrane binding, component A flattened on the membrane surface (FIG. 30c, FIG. 29).

In contrast, the membrane spanning orientation of A·B resulted in significant lipid remodeling by forming a toroidal lipid arrangement surrounding the pore perimeter (FIG. 30b, inset shows initial state) consistent with previous modeling. The toroidal arrangement shields the hydrophobic membrane core from the hydrophilic charged DNA helices (FIG. 31, FIG. 32). As a consequence of lipid re-arrangement, the bilayer broadens as indicated by an increased area per lipid headgroup (Table 6). The broadening is a consequence of geometric limitations imposed by lipid-packing and the maximization of interactions between the DNA phosphate backbone and lipid headgroups. Formation of the toroidal arrangement was accompanied by the alignment of the cholesterol anchors parallel to the fatty acid tail of the phospholipids (FIG. 30b), and an upward movement of A·B relative to the bilayer plane. Cholesterol moieties stabilized the surrounding bilayer as indicated by the reduced RMSF near the lipid anchors (FIG. 30d).

TABLE 6
Area per lipid (APL) values for the two membrane simulations.
APLs were calculated using the MEMBPlugin in VMD, and
averaged across the NpT production simulations, discarding
the initial 10% of the trajectory. The values compare
favourably with the literature values for POPC membranes,
but show an increased APL for the trajectory with the
membrane spanning DNA nanostructure.

| Trajectory | Area Per Lipid (Å²) |
|---|---|
| A · B | 74.49 ± 0.77 |
| A | 71.63 ± 0.07 |

Mapping of the channel lumen using MD simulations. MD simulations were used to elucidate the shape of the channel lumen. Cluster analysis was performed on the transmembrane A·B pore trajectory to generate a series of coordinates, which were then analyzed using the HOLE software—J. Mol. Graph. 1996, 14, 354-360—(FIG. 30e, FIG. 33, FIG. 34). The simulated map indicates that the pore has a dynamically changing lumen at the mid-section that ranges in diameter from 0.60±0.19 to 1.02±0.18 nm with an overall average of 0.83±0.14 nm. The pore is narrower at the top and bottom where the component duplexes are physically cross-linked. Nevertheless, these two regions also showed variation in diameter. A third narrowing of the channel is observed at the position of the cholesterol tags likely due to the bilayer-induced dynamic compression.

Triggered assembly of A·B on the membrane surface results in a functional nanopore. Following characterization of pore formation and its interaction with membranes, the pore activity was characterized. In particular, it was determined whether A·B formed on the membrane surface functioned as a bilayer-spanning nanopore (FIG. 35a,b). This question was addressed with single channel current recordings (SCCR). Pre-annealed A·B was examined first to establish the reference for the conductance of single nanopores. Addition of A·B to planar lipid bilayers resulted in a change in current to −54 pA at −50 mV (FIG. 35c). The current was recorded as a function of voltage for 16 independent insertions to confirm the presence of DNA pores. The ohmic current-voltage dependence between ±100 mV (FIG. 35d) matches the signal expected for the vertically symmetrical A·B as also found for other comparable DNA nanopores in the art. The analysis yielded a mean conductance of 0.70±0.27 nS (FIG. 35e). This is consistent with a theoretical conductance of 0.67 nS based on a nominal lumen diameter of 0.8 nm obtained from the lumen analysis by MD simulations (FIG. 30d).

After characterizing pre-annealed A·B, it was investigated whether triggered A·B assembly at membrane surface resulted in comparable pore characteristics compared to the directly assembled pore. For this investigation, the assembly-locked components AL^{A} and BL^{B} were incubated with planar lipid bilayer membranes. This did not lead to a current change indicating a lack of membrane puncturing (FIG. 35f). However, addition of keys K^{A} and K^{B} led to pore assembly on the membrane surface as confirmed by the characteristic increase in current to 71 pA at +90 mV (FIG. 35f) consistent with membrane inserted A·B. A small additional current step (FIG. 35f, asterisk) of 4.0 pA suggests small-scale local rearrangements of the duplexes upon pore insertion into the lipid bilayers. Further analysis from 8 independent insertions of trigger-assembled pores yielded a mean pore conductance of 0.69±0.12 nS (FIG. 35g) which is within error of the pre-assembled pore. The electrical recordings confirm that triggered assembly of A·B upon addition of keys results in a functionally identical nanopore to the directly assembled pore.

A·B transports molecular cargo across lipid bilayers. According to the MD simulations and SCCR experiments, the central pore lumen width of around 0.8 nm for the assembled nanostructure should support the flux of small-molecule cargo. To investigate this, molecular transport was probed across the A·B pore formed via direct assembly from components A and B, or via triggered assembly from locked components AL^{A} and BL^{B} and the addition of unlocking keys K^{A} and K^{B}. In the transport assay, the fluorophore sulforhodamine B (SRB), molecular weight of 558.7 g/mol, is encapsulated inside SUVs where it is contact quenched but increases in brightness when it effluxes across membrane pores into the ambient. As expected, there was no dye flux when the SRB-filled SUVs were incubated with individual components or the assembly-locked components in the absence of keys (FIG. 36a, FIG. 37).

Adding directly and trigger-assembled A·B at 400 nM to vesicles resulted in very similar dye effluxes (3.57±0.14% and 3.33±0.26%, respectively) (FIG. 36a) and lower fluxes when 200 nM pores were used (FIG. 37). The flux was 5.25±1.05% when 400 nM of pre-annealed pore A·B was used. The data indicate that the in situ assembled A·B results in a functional nanopore. The transport at around 3-5% is low and this is consistent with the expected slow diffusion of SRB (˜0.7 nm³²) across a pore with a relatively narrow lumen of 0.8 nm in diameter. Engineering a pore with a wider diameter lumen channel would allow for faster diffusion.

A·B forms synthetic Ca²⁺ permeable channels. The dye flux assay described above indicated that the narrow lumen of A·B makes it suitable for the transport of cargo even smaller than a fluorescence dye. Due to its small size (0.23 nm) and positive charge, Ca²⁺ was expected to transport even more efficiently across the pore than SRB, and thereby complement the SCCR data on ion transport. In the transport assay, the Ca²⁺-sensitive ratiometric dye, Fura-2, was encapsulated at 100 μM within SUVs, and 250 μM CaCl₂ was added to the ambient fluid. In the absence of pores, the SUV membranes were impermeable to Ca²⁺ as confirmed by fluorescence analysis (FIG. 38a). In addition, dynamic light scattering established that CaCl₂ did not disrupt vesicle integrity or significantly alter vesicle diameter (FIG. 38b). Upon addition of 25 nM pre-assembled pore (A·B)^{4C} resulted in considerable Ca²⁺ influx, reaching 79.2% signal after 10 min (FIG. 36b). An even higher flux of 90.3±3.1% (n=3) was achieved with 75 nM (A·B)^{4C} after only 70 s. In the absence of a pore, no significant Ca²⁺ influx was observed at all (0.94±0.13%, n=3).

### Conclusions

The present embodiment has pioneered the development of a synthetic DNA nanopore that forms by triggered assembly of inactive pre-pore components. Previous DNA pores were pre-formed in solution and integrated as complete pores into the membrane. The formation of the present pores proceeds either by direct assembly of the pore subunits or via activating the assembly-locked components with DNA keys (as an external stimulus) that reactivate pore assembly. Both routes produce the same assembly yield and pore function. Controlled pore formation from nucleic acid subunits and external triggers does not occur in nature. But the concept is related to biological protein pores which can assemble from membrane-tethered subunits. The oligomeric pores usually form via an intermediate non-membrane spanning pre-pore state which matures to the membrane puncturing pore via spontaneous conformational changes, such as the α-haemolysin pore or by protease-triggered changes, often found in the membrane-attack complexes. Nevertheless, it will be appreciated that the kinetics of protein pore assembly in vivo does not render the highly efficient ability of nucleic acid nanostructures to assemble into nanopores under the present conditions any less surprising and unexpected.

The bio-mimetic DNA pore described in this embodiment provided insight into processes underpinning controlled pore formation. Analyzing the affinity and kinetics of nanopore assembly determined the influence of membranes on molecular interaction and assembly. DNA hybridization was slowed down by an order of magnitude because assembly of the pore subunits requires a change from a membrane-tethered to a membrane-spanning orientation. Conversely, dissociation of the pore into the subunits is slowed down as this requires transition from the tethered to spanning orientation also reduces the structural flexibility of the DNA structures due to the stabilizing effect of the pore-surrounding lipid bilayer. Previous known nanopores do not involve a similar change in DNA association or dissociation. Hence, the present embodiment allows for the creation of a wide range of dynamic functional nanostructures at the membrane interface, and contributes to a further understanding of molecular processes at membranes. By aiming to create synergies between chemistry and the life sciences, this embodiment makes feasible a wide range of customizable nanodevices for use in biomedicine, synthetic biology, sensor technology and chemical biology.

### 1. Experimental Information

### 1.1. Materials

Unmodified, fluorophore-labeled, and cholesterol-modified DNA oligonucleotides were purchased from Integrated DNA Technologies on a 100 nmol scale with HPLC purification. 1,2-dioleoyl-sn-glycero-3-phosphocholine (DOPC), 1,2-dioleoyl-sn-glycero-3-phosphoethanolamine (DOPE), 1-palmitoyl-2-oleoyl-sn-glycero-3-phosphocholine (POPC) and 1,2-diphytanoyl-sn-glycero-3-phosphocholine (DPhPC) were procured from Avanti Polar Lipids (US). All other reagents and solvents were purchased from Merck (UK) unless specified.

### 1.2. DNA Assembly

Equimolar mixtures of DNA oligonucleotides (1 μL each, stock concentration of 100 μM) (Table S2 for composition of DNA pores and components) were dissolved at 1 μM in a buffer solution of either buffer A (1×PBS; 137 mM NaCl, 2.7 mM KCl, 10 mM Na₂HPO₄, 1.8 mM KH₂PO₄, pH 7.4), buffer B (300 mM KCl, 15 mM Tris-HCl, pH 7.4) or buffer C (12 mM MgCl₂ in 0.6×TAE (40 mM Tris, 20 mM acetic acid), pH 7.4) to a final volume of 100 μL. Folding was achieved on a BioRad T100 Thermocycler (UK) using a program involving heating to 95° C. and holding for 0.5 min, then cooling to 75° C. within 5 min, holding for 1 min before cooling to 4° C. at a rate of 0.5° C. per 1 min. Samples were stored at 4° C. for up to 1 week.

### 1.3. Page

The assembled DNA nanostructure and component DNA oligonucleotides were analyzed with commercial 10% polyacrylamide gels (BioRad, UK) in 1×TBE buffer (100 mM Tris, 90 mM boric acid, 1 mM EDTA, pH 8.3). For gel loading, a solution of the DNA nanopores (2 μL, 1 μM) was mixed with folding buffer (13 μL, 2 mM MgCl₂ in 0.6×TAE, pH 7.4) and 6× gel loading dye (5 μL, New England Biolabs, UK). Gels were run at 115 V for 90 min at 4° C. The gel bands were visualized by staining with ethidium bromide and UV illumination. A 100 bp marker (New England Biolabs, UK) was used as a reference standard.

### 1.4. Agarose Gel Electrophoresis

The assembled DNA nanostructures and component DNA oligonucleotides were analyzed with 2-3% agarose (Invitrogen, UK) gels in 1×TAE buffer (40 mM Tris, 20 mM acetic acid, 1 mM EDTA, pH 8.3). For gel loading, a solution of the DNA nanostructure (2 μL, 1 μM) was mixed with folding buffer (13 μL) and 6× gel loading dye (5 μL, New England Biolabs, UK). The gel was run at 60 V for 60 min at 4° C. The gel bands were visualized by staining with ethidium bromide and UV illumination. A 100 bp marker (New England Biolabs, UK) was used as a reference standard.

### 1.5. Preparation of Small Unilamellar Vesicles (SUVs)

DPhPC (100 μL, 10 mM) or POPC (100 μL, 10 mM) in chloroform was added to a 5 mL round bottom flask. The solvent was removed using a rotary evaporator (Buchi, Newmarket, UK) to yield a thin film, which was further dried under high vacuum (Buchi, Newmarket, UK) for 1 h. The lipid was re-suspended in 1 mL of either buffer A or buffer B. The solution was sonicated for 20 min at 30° C. and then equilibrated for 1 h before being extruded 25 times through a 0.1 μm polycarbonate membrane (Avanti Polar Lipids, US) using the extruder kit (Avanti Polar Lipids, US). SUVs were then stored at 4° C. and used within 48 h.

### 1.6. Melting Temperature (Tₘ) Analysis Using UV-Vis Spectroscopy

UV melting profiles were obtained using a 10 mm quartz cuvette (Hellma Analytics, Southend-on-Sea, UK) in a Varian Cary 300 Bio UV-vis spectrophotometer (Agilent, Cheadle, UK) equipped with a Peltier element (Agilent, Cheadle, UK). Samples were analyzed at 200 nM and SUVs composed of DPhPC at 200 μM lipid concentration. Samples were analyzed by monitoring the change in absorbance at 260 nm as the temperature was increased from 20 to 80° C. at a rate of 1° C./min. Melting profiles were then background corrected, and the 1st derivative calculated to identify the Tₘ.

### 1.7. Electrophoretic Mobility Shift Assay

For binding titrations, component A^{ΔC} or A (5 μL, 1 μM) was mixed with component B^{ΔC} or B (1 μM stock) in buffer A yielding concentrations of 0 to 0.5 μM in a final volume of 20 μL. In the case of A-SUV vs B^{ΔC} or B, component A (5 μL, 1 μM) was first added to SUVs (5 μL, 100 nm, 16.7 nM). After incubation for 30 min at 30° C., 6× gel loading dye (5 μL, New England Biolabs, Hitchin, UK) was added, the samples were mixed and loaded onto a thermally equilibrated 2-3% agarose gel. The gel was run in 1×TAE buffer, pH 8.3 at 60 V for 60 min at 4° C. Staining and molecular markers were as described in section 1.4. Band intensities were analyzed using ImageJ and normalized as (1−(I_{A}−I_{background})). The normalized intensities were then fit to a Langmuir curve to determine the K_{d}.

Kinetic assembly titrations, component A^{ΔC} (5 μL, 1 μM) was mixed with B^{ΔC} (5 μL, 1 μM) in buffer A to a final volume of 20 μL. Samples were incubated at 30° C. for 0, 1, 5, 10, 15, 20, 25 and 30 min while shaking at 500 rpm. Samples were prepared in reverse time order and after all samples were prepared, samples were crashed in ice to arrest pore formation. For assembly locked components A^{ΔC}L^{A} vs B^{ΔC}L^{B} the keys, K^{A} and K^{B} (1 μL, pre-mixed, 5 μM) were also added to each timepoint. Samples were mixed with 6× gel loading dye (5 μL) and then loaded onto thermally equilibrated 10% PAGE. The gel was run in 1×TBE buffer at 115 V for 90 min at 4° C. Staining and molecular markers were as described in section 1.3.

### 1.8. FRET Assay on Pore Assembly

For binding titrations, the assembly of A·B was investigated using a fluorescence spectrophotometer (Cary Eclipse, Agilent, Cheadle, UK). To a plastic Eppendorf tube was added A^{ΔC}, A or AL^{A} (12 μL, 1 μM), B^{ΔC}, B or BL^{B} (0 μL, 1.2 μL, 2.4 μL, 6 μL, 12 μL, 24 μL; 1 μM), SUVs (0 μL, 6 μL; 1 mM lipid, 7.22 nM SUV) and buffer B to a final volume of 120 μL. The tube was then incubated at 30° C. for 30 min while shaking at 750 rpm. The combined solution was then added to a 10 mm quartz cuvette (Hellma Analytics, Southend-on-Sea, UK), which was placed in the fluorescence spectrophotometer and scanned (ex_{545 nm}, em_{555-725 nm}). Pre-folded A·B was used as a control for maximum assembly. Where SUVs were used, A or AL^{A} and SUVs were mixed and left to bind for 10 min prior to addition of B^{ΔC}, B or BL^{B}. The emission intensity of the donor (Cy3) were normalized between A^{ΔC} or A and a pre-folded control pore (A·B)^{ΔC}, (A·B)^{1C}, or A·B. A 1:2 of A:B was used as an internal control. Due to the ability of the donor (Cy3) to donate to multiple acceptor (Cy5) molecules, this was set to the same binding level as the pre-assembled control and was used as an anchor point for K_{d} determination.

For kinetic assembly, the assembly of A·B was investigated by monitoring Cy3 emission (ex_{550 nm}, em_{570 nm}) using a fluorescence spectrophotometer (Cary Eclipse, Agilent, Cheadle, UK). To a 10 mm quartz cuvette (Hellma Analytics, Southend-on-Sea, UK), A^{ΔC} or A (2.5 μL, 1 μM) was added to SUVs (0 μL, 1.25 μL; 1 mM lipid, 7.22 nM SUV) and buffer B (97.5, 96.25 μL) and the signal left to stabilize for 5 min. Then, B^{ΔC} or B (50 μL, 1 μM) was rapidly added and mixed. Pore formation was monitored for 1 h. Where SUVs were used, A and SUVs were mixed and left to bind for 10 min prior to the start of the run.

FRET Efficiency (E) calculations were achieved using the equation (2):

$$E = 1 - \frac{{I}_{DA}}{{I}_{D}} \qquad ( 2 )$$

Where I_{DA} is the donor intensity in the presence of the donor and acceptor; I_{D} is the donor intensity in the absence of the acceptor.

The inter-fluorophore (Cy3-Cy5) distance was calculated using equation (3):

$$E = \frac{1}{1 - {( \frac{r}{{R}_{0}} )}^{6}} \qquad ( 3 )$$

E stands for FRET efficiency, r is the donor-acceptor separation distance, R₀ is the Förster distance where E=50%.

### 1.9. Dual-Color FCCS Analysis to Measure the K_{d} of Pore Assembly

Dual-color fluorescence cross-correlation measurements were carried out on a commercial laser scanning microscope (ConfoCor 3, Carl Zeiss, Jena, Germany) equipped with a 40× water immersion objective. A^{ΔC} and B^{ΔC} were labeled with the spectrally non-overlapping fluorophores Alexa488 and Alexa647, respectively. Cross-correlation measurements were performed using a 635 nm secondary dichroic mirror with a 505-540 nm bandpass filter in the green channel, and a 650 nm longpass filter in the red channel. Laser power was adjusted such that the brightness ratio of Alexa647 to Alexa488 was roughly 3:1.

At a 1:1 binding stoichiometry, the bound fraction of either ^{Alexa488}A^{ΔC} (X_{g}) or ^{Alexa647}B^{ΔC} (Xᵣ) can be calculated from the number of double labelled particles (N^{rg}) relative to the total number of each particle (N^{g} for ^{Alexa488}A^{ΔC} or N^{r} for ^{Alexa647}B^{ΔC}) using equations 4 and 5:

$${X}_{g} = \frac{{N}^{rg}}{{N}^{g}} \qquad ( 4 )$$

$${X}_{r} = \frac{{N}^{rg}}{{N}^{r}} \qquad ( 5 )$$

The number of particles of ^{Alexa488}A^{ΔC} or ^{Alexa647}B^{ΔC} detected in the green or red channel, respectively, was obtained from the fit of the respective autocorrelation (for N^{g} and N^{r}) and cross-correlation (for N^{rg}) function G(τ) by the one-component model for 2D translational diffusion shown in equation 6:

$$G ( τ ) = 1 + \frac{1}{N ( 1 + τ / {τ}_{D} )} \qquad ( 6 )$$

where τ_{D} represents the diffusion time through the confocal volume. The fraction of bound particles, X_{g} and Xᵣ, was corrected by accounting for the difference in size of the green and red detection volumes, V_{g} and Vᵣ, by using the following formulas:

$${X}_{g / v} = {X}_{g} ⋆ \frac{{V}_{g}}{{V}_{eff}} \qquad ( 7 )$$

$${X}_{r / v} = {X}_{r} ⋆ \frac{{V}_{g}}{{V}_{eff}} \qquad ( 8 )$$

where V_{eff} represents the effective cross-correlation volume, which is defined by the following equation:

$${V}_{eff} = {π}^{3 / 2} ⋆ {( \frac{{ω}_{g}^{2} + {ω}_{g}^{2}}{2} )}^{\frac{3}{2}} ⋆ S \qquad ( 9 )$$

where S is a structural parameter, which was set equal for both channels, S_{g}=Sᵣ=6, and ω is the respective lateral radius of the confocal volume, ω_{g} or ωᵣ, of the green or red confocal volume. Confocal volumes, V, in the red and green channels were calculated as:

$$V = {π}^{3 / 2} ⋆ {ω}^{3} ⋆ S \qquad ( 10 )$$

The lateral radii, ω_{g} and ωᵣ, we obtained from calibration experiments measuring labels with known diffusion coefficients (D), Rhodamine 6G (Merck, Germany) and Alexa647 maleimide (Jena Bioscience, Germany), using the equation:

$${ω}^{2} = 4 D {τ}_{D} \qquad ( 11 )$$

On each day that data were collected, the maximum achievable cross-correlation was determined using a DNA-duplex carrying both an Alexa488 and Alexa647 fluorophore (IDT, USA) to represent 100% binding.

For binding measurements, ^{Alexa488}A^{ΔC} was mixed with ^{Alexa647}B^{ΔC} in buffer B, to a final volume of 60 μL. In other measurements, the concentration of ^{Alexa647}B^{ΔC} was varied from 0.5 nM to 300 nM, while the concentration of ^{Alexa488}A^{ΔC} (7.5 μL, 100 nM) remained constant. The samples were then incubated at 30° C. for 30 min with shaking. After incubation, each ratio mixture was measured separately. Measurements where ^{Alexa647}B^{ΔC} remained constant (9.6 μL, 100 nM), while the concentration of ^{Alexa488}A^{ΔC} was varied.

To determine K_{d}, the resulting volume-corrected fractions of bound particles X_{g/v} and X_{r/v} were fitted to a Langmuir isotherm:

$$X = A ⋆ \frac{C}{C + {K}_{d}} \qquad ( 12 )$$

where C represents concentration of the varied component.

By design, a do-FCCS experiment shows the saturation value below 100%, and a slight overestimation of the cross-correlation is observed in the red channel (FIG. 10a). The former originates from the non-complete overlapping of the confocal volumes for the red and green lasers. The latter stems from the bleed-through of the green-labelled particles into the red channel at higher ratios of green-to-red particles, which is pronounced at ratios higher than 10:1.4 To minimize the effect, we performed before every FCCS experiment a control measurement using a cross-correlation standard. This is a fluorescently double labelled DNA-duplex. Within 5% accuracy, the control measurements showed the same deviation from 100% saturation level, and the same ratio of saturation levels between the red and the green channels.

### 1.10. Preparation of Giant Unilamellar Vesicles (GUVs)

A solution of POPC lipids (5 μL, 10 mM in chloroform) was added to an indium tin-oxide (ITO) coated glass slide. Within 5 min the solvent evaporated, and a dried lipid film was formed. The glass slide was then inserted in a vesicle prep device (Nanion Technologies, Munich, Germany). An O-ring was added around the patch. Sucrose (300 μL, 1 M in water) was added to the lipid film patches confined by the O-ring. Finally, another ITO glass slide was applied from the top, resulting in a sealed chamber. An alternating electric field was applied between the two slides by using a voltage program of 3 V, 5 Hz for 120 min. The solution was collected and stored at 4° C.

### 1.11. Confocal Laser Scanning Microscopy

A GUV suspension (10 μL, 130 μM lipid concentration) was added to a FluoroDish (World Precision Instruments, Hitchin, UK) with buffer (500 μL, 1×TAE, 500 mM NaCl, pH 8.1). The solution gently mixed. After adding component A (10 μL, 1 μM) to the dish, the solution was mixed thoroughly and left for 10 min to ensure membrane binding. Component B^{ΔC} (10 μL, 1 M) was then added following by mixing of the solution. The mixture was left for 5 min to allow the GUVs sink to the bottom of the dish. The FluorDish (World Precision Instruments, Hitchin, UK) was placed under the microscope and GUVs were located by visualization using a 96× optical zoom. The sample was then viewed through the brightfield and at 570 nm for ^{Cy3}A and 670 nm for ^{Cy5}B and images were acquired.

### 1.12. Preparation of Planar Lipid Bilayers on a Glass Slide and smFRET and Single Particle Tracking

Planar lipid bilayers were formed on glow discharged glass slides provided by Oxford NanoImaging (Oxford, UK). SUVs composed of DPhPC in buffer A (15 μL, 1 mM) were placed onto the support and left for 15 min. Some solution (˜5 μL) was then supplanted with H₂O and left for 1-2 min. This was repeated 3×. After the 3^{rd} wash with H₂O, the solution was washed with buffer A. Slides were used within 1 h and topped up with buffer A as necessary. smFRET and single particle tracking was performed using a Nanolmager S (Oxford Nanolmaging, Oxford, UK) by Jon Shewring from Oxford Nanolmaging. Structures were added (1 μL, 1 nM in buffer A) to planar lipid bilayers composed of DPhPC on glass slides.

### 1.13. Enzyme Digestion Assay

A solution of the lipid DPhPC (100 μL, 10 mM) in chloroform was added to a 5 mL round bottom flask. The solvent was removed using a rotary evaporator (Buchi, Newmarket, UK) to yield a thin film. The lipid was re-suspended in buffer B (1 mL), sonicated for 20 min at 30° C. and then equilibrated for 3 h. (A·B)^{ΔC}, (A·B)^{1C} or A·B (2 μL, 1 μM), nuclease-free water (58 μL) and 2×Bal-31 buffer (60 μL; 40 mM Tris-HCl, 1.2 M NaCl, 24 mM MgCl₂, 24 mM CaCl₂, 2 mM EDTA) were added to a 10 mm quartz cuvette (Hellma Analytics, Southend-on-Sea, UK). For assays with vesicles, (A·B)^{ΔC}, (A·B)^{1C} or A·B (2 μL, 1 μM) were first incubated with LUVs (18 μL, 1 mM DPhPC) for 1 h then added to the 10 mm quartz cuvette with nuclease-free water (40 μL) and 2×Bal-31 buffer (60 μL, New England Bioscience, Hitchin, U.K.). Fluorescence was monitored using a Varian Cary Eclipse fluorescence spectrophotometer (Agilent, Cheadle, UK) at 570 nm and excited at 555 nm. After 5 min, Bal-31 (0.75 μL, New England Bioscience, Hitchin, U.K.) was added, and the fluorescence emission was monitored for 15 min.

### 1.14. Circular Dichroism

Solution-phase circular dichroism (CD) spectroscopy was performed on a Jasco-810 spectropolarimeter (Kromatec Ltd, Great Dunmow, UK). A micro-volume quartz Couette flow cell with ˜0.5 mm annular gap and quartz capillaries were used (Kromatec Ltd, Great Dunmow, UK). CD spectra were acquired for DNA nanopores (1.4 μM) between 320-190 nm.

### 1.15. Linear Dichroism

Solution-phase flow linear dichroism (LD) spectroscopy was performed on a Jasco-810 spectropolarimeter (Kromatec Ltd, Great Dunmow, UK) using a photo elastic modulator ½ wave plate. A micro-volume quartz Couette flow cell with ˜0.5 mm annular gap and quartz capillaries were used (all from Kromatec Ltd, Great Dunmow, UK). Molecular alignment was achieved by applying the constant flow of the sample solution between two coaxial cylinders, a stationary quartz rod and a rotating cylindrical capillary. LD spectra were acquired with laminar flow obtained by maintaining the rotation speed at 3000 rpm and processed by subtracting non-rotating baseline spectra. DNA nanopores were assayed at 1.4 μM and SUVs composed of POPC at 500 μM lipid concentration.

### 1.16. Simulation Preparation

DNA nanopore A·B, and component A were recreated in caDNAno, then converted to all atom models in python. The poly-thymine linker regions at the pore termini were then constructed using the MolSoft ICM software suite. TEG-Cholesterol lipid anchors were parametrized using cgenff⁷ and attached using pyMol. CHARMM36 compatible topology files were then generated using psfgen. Initial structures of A·B and A were minimized in a vacuum for 10,000 steps (2 fs), then simulated for 100,000 steps (2 ns) using an elastic restraint network derived from the ENRG webserver.

DNA nanopore A·B and component A were simulated in 1 M KCl and TIP3 water prepared in VMD.

Nanopore A·B was simulated in 13×11×15 nm box totaling 437 k atoms and component A was simulated in a box of 16×14×19 nm totaling 6.5 k atoms. A 1 ns NpT equilibration was run to equilibrate box size and pressure before a 50 ns NvT equilibration to further relax the DNA nanostructures. Production simulations were then run in in the NpT ensemble.

For simulations of membrane tethered component A and membrane-inserted A·B, VMD was used to generated membranes and orient the DNA nanostructures while maintaining favorable cholesterol orientations. The orientation of each structure was informed by experimental data derived from linear dichroism. The membrane-spanning nanopore A·B was simulated in a 12×12×12 nm box of 1 M KCl, bisected by a bilayer composed of POPC lipids for a total of 141 k atoms. The membrane-tethered component A was simulated in a 15×15×16 nm box in the same conditions totaling 303 k atoms. While the fixed atom restraints were placed on all atoms except those of the lipid tails, which were then thermally equilibrated over 0.5 ns of dynamics in the NvTensemble as the temperature was increased to 301 K. Fixed atom restraints were replaced with harmonic restraints, with a spring constant of 1 kcal/mol/Å², on the heavy atoms of the DNA phosphate backbones. Simulation box size and pressure were equilibrated in the NpT ensemble for 3.5 ns, with harmonic restraints being lowered by 0.5 kcal/mol/Å² every 0.5 ns. Unrestrained dynamics in the NvT ensemble allowed the system to fully equilibrate, and production simulations were performed in the NpT ensemble.

Production simulations were performed at 301K and 1.013 bar pressure, maintained with the Langevin thermostat and the Nosé-Hoover Langevin piston method. Simulations were performed in NAMD, a smooth switching algorithm with a switch distance of 8 Å, a cut off of 10 Å and a pair list distance of 12 Å was implemented for van der Waals interactions. A 2 fs time step was used and hydrogen bond lengths were constrained using the SETTLE and SHAKE algorithms. Particle Mesh Ewald electrostatics were computed over a cubic grid with a 1.0 Å spacing and periodic boundary conditions. Equilibration simulations were performed on a on a single GPU 1080Ti workstation and production runs were performed in parallel on 850 CPU cores of the UCL Grace HPC facility.

### 1.17. Simulation Analysis

Analysis was performed using GROMACs and VMD tools on the production simulations, after discarding the initial 10 ns, graphs were prepared using ggplot and RStudio.

### 1.17.1. RMSF¹⁰

gmx_covar and gmx_aneig were used to investigate the ten top quasi-harmonic modes of root mean squared fluctuations (RMSF) of the DNA backbone heavy atoms, averaged per-residue, to interrogate structural dynamics of the DNA nanostructures while accounting for thermal noise and stochastic motion.

### 1.17.2. Clustering

gmx_cluster was used to prepare snapshots of the membrane spanning A·B DNA nanostructure trajectory. Clustering was performed with a cut-off of 0.35 nm using the gromos method.

### 1.17.3. Lumen Analysis

Clustered coordinates were analyzed using HOLE, with a channel-end radius of 0.8 nm and a sampling distance of 0.25 nm. To account for asymmetry of the DNA nanostructure, coordinates were then rotated and analyzed again.

### 1.17.4. Lipid Analysis

gmx_gangle was used to measure the angle of phosphate and nitrogen atoms in the lipid head groups, split by lipid leaflet, compared to the bilayer normal, over the initial equilibration simulations. Production simulations were analyzed using gmx_rms, and the VMD plugins density_profile_too and MEMBPLUGIM to determine lipid RMSF, average lipid density and area-per-lipid, respectively.

### 1.18. Nanopore Current Recordings

Single-channel current measurements were achieved using an integrated chip-based, parallel bilayer recording setup (Orbit Mini, Nanion Technologies, Munich, Germany) with multielectrode-cavity-array (MECA) chips (IONERA, Freiburg, Germany). Bilayers were formed from DPhPC (10 mg/mL in octane). The electrolyte solution was 1 M KCl and 10 mM HEPES, pH 7.4. To achieve pore insertions, a 2:1 mixture of nanopore A·B and 0.5% OPOE (n-octyloligooxyethylene, in 1 M KCl, 10 mM HEPES, pH 7.4) was added to the cis side of the bilayer. Successful insertion was observed by detecting current steps. For triggered assembly, membranes were preincubated with AL^{A} and BL^{B} and 0.5% (v/v) OPOE. After 10 minutes, key strands were added and successful insertions were observed by detecting current steps. The current traces were not Bessel-filtered and were acquired at 10 kHz using Element Data Recorder software (Element s.r.I., Cesena, Italy). Single-channel analysis was performed using Clampfit software (Molecular Devices, Sunnyvale, CA, USA).

### 1.19. Preparation of Fluorophore-Filled SUVs and Dye Release Assay

A solution of DPhPC lipids (100 μL, 10 mM) in chloroform was added to a 5 mL round bottom flask. The solvent was removed using a rotary evaporator (Buchi, Newmarket, UK) to yield a thin film, which was further dried under high vacuum (Buchi, Newmarket, UK) for 1 h. The lipid was re-suspended in buffer A containing the fluorophore sulforhodamine B (SRB, 50 mM), sonicated for 20 min at 30° C. and then equilibrated for 3 h at 4° C. SUVs were then extruded 25 times through a 100 nm polycarbonate membrane (Avanti Polar Lipids, US) using an extruder kit (Avanti Polar Lipids, US). The non-encapsulated SRB was removed using a NAP-25 column (Cytivia, UK) and SUVs were exchanged into buffer D (0.2 M KCl, 10 mM Tris pH 7.4). Purified SUVs were used within 48 h and gently resuspended immediately prior to use.

For the release assays, A·B was folded at 1 μM in buffer C using the 15 h folding protocol while components A and B were folded at 2 μM. The SUV suspension with encapsulated SRB (10 μL) and buffer D (110 μL, 80 μL) were added to a 10 mm quartz cuvette (Hellma Analytics, Southend-on-Sea, UK). Fluorescence was monitored using a Varian Cary Eclipse fluorescence spectrophotometer (Agilent, Cheadle, UK) at 586 nm and excited at 565 nm. After 5 min, A, A+B, A·B, AL^{A}+BL^{B} or AL^{A}+BL^{B}+K^{A}+K^{B} (30 μL, or 60 μL; 1 μM in buffer C) was added to a final volume of 150 μL. After 55 min of monitoring fluorescence, samples were mixed with a 1% (v/v) solution of Triton X-100 (10 μL) to lyse all vesicles to identify maximum SRB release. Maximum fluorescence emission and the fluorescence prior to addition of A·B (or components) were used to calculate the extent of release as %.

### 1.20. Preparation of Fura-2-Filled SUVs and Ca²⁺ Influx Assay

A solution of the lipid POPC (100 μL, 10 mM) in chloroform was added to a 5 mL round bottom flask. The solvent was removed using a rotary evaporator (Buchi, Newmarket, UK) to yield a thin film, which was further dried under high vacuum (Buchi, Newmarket, UK) for 1 h. The lipid was re-suspended in buffer E (500 mM NaCl, 100 mM HEPES, pH 7.4) containing the fluorophore Fura-2 (100 μM). The solution was sonicated for 20 min at 30° C. and then equilibrated for 3 h. SUVs were extruded 25 times through a 100 nm polycarbonate membrane (Avanti Polar Lipids, US) using an extruder kit (Avanti Polar Lipids, US). The non-encapsulated dye was removed using Illustra MicroSpin S-400 spin columns (Cytivia, UK). SUVs were then subjected to dynamic light scattering with a Malvern Zetasizer Nano S (Malvern Pananalytical, Malvern, UK) to confirm the vesicles' diameter. Purified SUVs were used within 48 h and gently resuspended immediately prior to use. For Ca²⁺ influx assays, the SUV suspension with encapsulated Fura-2 (30 μL) and buffer E (138.3 μL, 145.8 μL, 148.33 μL) were added to a 10 mm quartz cuvette (Hellma Analytics, Southend-on-Sea, UK Fluorescence was monitored using a Varian Cary Eclipse fluorescence spectrophotometer (Agilent, UK) at 510 nm and excited at 340 and 380 nm. After 2.5 min, CaCl₂ (16.7 μL, 3 mM in H₂O) was added and allowed to stabilize for a further 2.5 min. At 5 min, (A·B)^{4C} (15 μL, 7.5 μL, 5 μL, 1 μM in buffer B) was added to a final volume of 200 μL. After 30 min of monitoring fluorescence, samples were mixed with a 1% solution (v/v) of Triton X-100 (10 μL) to lyse all vesicles to identify maximum Ca²⁺ influx. Ca²⁺ influx was monitored as the ratio of the change in emission at each excitation wavelength as a ratio of 340/380 nm. The maximum 340/380 nm ratio following addition of Triton X-100 was used to normalize all traces.

TABLE 6
Names, modifications and sequences of DNA
oligonucleotides used for folding of
nanostructures A, B, A·B and variants

| | SEQ | |
|---|---|---|
| | ID | |
| ID | NO: | Sequence 5′ → 3′ |
| A₁ | 1 | ATTAGCGAACGTGGATTTTGTCCGACATCGGCAAGCT |
| | | CACTTTTTCGACT |
| A₂ | 2 | TTGCCGATGTCGGACTTTTGAACGATCTTCGCCTGCT |
| | | GCGTTTTGTGAGC |
| B₁ | 3 | AGGCGAAGATCGTTCTTTTCCTGCACGTCCAACTGGT |
| | | CAGTTTTCGCAGC |
| B₂ | 4 | AGTTGGACGTGCAGGTTTTTCCACGTTCGCTAATAGT |
| | | CGATTTTCTGACC |
| L^{A} | 5 | GGACCATGGTTCCACGTTCGCTAATAGTCGA |
| L^{B} | 6 | TGGTCAAGCAGAACGATCTTCGCCTGCTGCG |
| K^{A} | 7 | TCGACTATTAGCGAACGTGGAACCATGGTCC |
| K^{B} | 8 | CGCAGCAGGCGAAGATCGTTCTAGCTTGACC |
| A₁ | 9 | Sequence of A₁ carrying a cholesterol |
| (chol) | | via tri(ethylene glycol) |
| | | TEG linker at the 3′ terminus |
| A₂ | 10 | Sequence of A₂ carrying a cholesterol |
| (chol) | | via a TEG linker at the 3′ terminus |
| B₁ | 11 | Sequence of B₁ carrying a cholesterol |
| (chol) | | via a TEG linker at the 3′ terminus |
| B₂ | 12 | Sequence of B₂ carrying a cholesterol |
| (chol) | | via a TEG linker at the 3′ terminus |
| ^{Cy3}A1 | 13 | ^{Cy3}ATTAGCGAACGTGGATTTTGTCCGACATCGGCAAG |
| | | CTCACTTTTTCGACT |
| ^{Cy5}B1 | 14 | ^{Cy5}AGGCGAAGATCGTTCTTTTCCTGCACGTCCAACTG |
| | | GTCAGTTTTCGCAGC |
| ^{Alexa488}A1 | 15 | ^{Alexa488}ATTAGCGAACGTGGATTTTGTCCGACATCGGCA |
| | | AGCTCACTTTTTCGACT |
| ^{Alexa647}B1 | 16 | ^{Alexa647}AGGCGAAGATCGTTCTTTTCCTGCACGTCCAAC |
| | | TGGTCAGTTTTCGCAGC |

Cy3X = Cy3 fluorophore; Cy5X = Cy5 fluorophore; Alexa488X = Alexa488 fluorophore; Alexa647X = Alexa647 fluorophore

TABLE 7
Names and strand compositions of structures used for the
exemplary DNA nanopore with biomimetic triggered assembly

| | Composition |
|---|---|
| A^{ΔC} | A₁, A₂ |
| B^{ΔC} | B₁, B₂ |
| A | A₁, A₂ (chol) |
| B | B₁, B₂ (chol) |
| ^{Cy3}A^{ΔC} | ^{Cy3}A₁, A₂ |
| ^{Alexa488}A^{ΔC} | ^{Alexa488}A₁, A₂ |
| ^{Cy3}A | ^{Cy3}A₁, A₂ (chol) |
| ^{Cy5}B^{ΔC} | ^{Cy5}B₁, B₂ |
| ^{Alexa647}B^{ΔC} | ^{Alexa647}B₁, B₂ |
| ^{Cy5}B | ^{Cy5}B₁, B₂ (chol) |
| A^{ΔC}L^{A} | A₁, A₂, L^{A} |
| B^{ΔC}L^{B} | B₁, B₂, L^{B} |
| AL^{A} | A₁, A₂ (chol), L^{A} |
| BL^{B} | B₁, B₂ (chol), L^{B} |
| (A · B)^{ΔC} | A₁, A₂, B₁, B₂ |
| (A · B)^{1C} | A₁, A₂ (chol), B₁, B₂ |
| A · B | A₁, A₂ (chol), B₁, B₂ (chol) |
| (A · B)^{4C} | A₁ (chol), A₂ (chol), B₁ (chol), B₂ (chol) |

TABLE 8
Names, modifications, and sequences of DNA
oligonucleotides used for folding R, S
and SNP.

| | SEQ | |
|---|---|---|
| | ID | |
| ID | NO: | Sequence 5′ → 3′ |
| R^{ΔC} | 17 | TAGTCGATTTTATCCATGCA |
| R | 18 | Sequence of R^{ΔC} carrying a |
| | | cholesterol via a tri(ethylene) |
| | | glycol (TEG) linker at the |
| | | 3' terminus |
| ^{Cy3}R^{ΔC} | 19 | Cy3TAGTCGATTTTATCCATGCA |
| ^{Cy3}R | 20 | Sequence of ^{Cy3}R^{ΔC} carrying a |
| | | cholesterol via a TEG linker |
| | | at the 3' terminus |
| S | 21 | TGCATGGATAAAATCGACTA |
| ^{Cy5}S | 22 | TGCATGGATAAAATCGACTATTTTT^{Cy5} |
| 1 | 23 | ACA GGA TTT TCG CCT GCT GGG |
| | | GCA AAC CAG CGT GGA CCG CTT |
| | | TTT TGG CTA TTC TTT TGA TTT |
| | | ATA AGG GAT TTT GCC GAT TTC |
| | | GGA A |
| 2 | 24 | CAA CTC TCT CAG GGC CAG GCG |
| | | GTG AAG GGC AAT CAG CTG TTG |
| | | TTT TCA ACA GCA TCC TGT TTC |
| | | CGA AAT |
| | | CGG CAT TAA AGA CCA GCT G |
| 3 | 25 | TCT CAC TGG TGA AAA GAA AAA |
| | | CCA CCC TGG CGC CCA ATA CGC |
| | | TTT TTC CCC GCG CGT TGG CCG |
| | | ATT CAT TAA TGC AGC TGG CAC |
| | | GAC A |
| 4 | 26 | GGC GAA ATG ATT GCT TTC ACC |
| | | AGT GAG ATG TCG TGA CGT GGA |
| | | TTT TTC CAC GTT CTT TAA TAG |
| | | TGG ACT CTT GTT CCA AAC TGG |
| | | AAC A |
| 5 | 27 | TGT TCC AAA TAG CCA AGC GGT |
| | | CCA CGC TCC CTG AGG GGC GCC |
| 6 | 28 | CAT TAA TTT TTT CTC CTT CAC |
| | | CGC CTG GGG TTT GCT TAT AAA |
| 7 | 29 | AGG GTG GGA ATC GGA CAA GAG |
| | | TCC ACT AAA ATC CCC CCA GCA |
| 8 | 30 | TCA AAA GGT TTG GAC CAA CGC |
| | | GCG GGG AGC GTA TTA GAG TTG |
| 5-ext | 31 | TGC ATG GAT AAA ATC GAC TAT |
| | | TTT TGT TCC AAA TAG CCA AGC |
| | | GGT CCA CGC TCC CTG AGG GGC |
| | | GCC |
| ^{Cy5}5-ext | 32 | TGCATGGATAAAATCGACTATTTTT^{Cy5} |
| | | GTTCCAAATAGCCAAGCGGTCCACGC |
| | | TCCCTGAGGGGCGCC |

X^{Cy3} = Cy3 fluorophore; X^{Cy5} = Cy5 fluorophore

TABLE 9
Names and strand compositions of structures
used for the model system

| | Composition |
|---|---|
| NP | 1, 2, 3, 4, 5, 6, 7, 8 |
| S_{NP} | 1, 2, 3, 4, 5-ext, 6, 7, 8 |
| ^{Cy5}S_{NP} | 1, 2, 3, 4, ^{Cy5}5-ext, 6, 7, 8 |
| R^{ΔC} · S | R^{ΔC}, S |
| R · S | R, S |
| R^{ΔC} · S_{NP} | R^{ΔC}, 1, 2, 3, 4, 5-ext, 6, 7, 8 |
| R · S_{NP} | R, 1, 2, 3, 4, 5-ext, 6, 7, 8 |
| ^{Cy3}R^{ΔC} · ^{Cy5}S | ^{Cy3}R^{ΔC}, ^{Cy5}S |
| ^{Cy3}R · ^{Cy5}S | ^{Cy3}R, ^{Cy5}S |
| ^{Cy3}R^{ΔC} · ^{Cy5}S_{NP} | ^{Cy3}R^{ΔC}, 1, 2, 3, 4, ^{Cy5}5-ext, 6, 7, 8 |
| ^{Cy3}R · ^{Cy5}S_{NP} | ^{Cy3}R, 1, 2, 3, 4, ^{Cy5}5-ext, 6, 7, 8 |

### REFERENCES FOR MATERIALS AND METHODS

(1) Magde, D.; Elson, E. L.; Webb, W. W. Fluorescence correlation spectroscopy. Ii. An experimental realization. Biopolymers 1974, 13, 29-61.
(2) Werner, S.; Ebenhan, J.; Haupt, C.; Bacia, K. A quantitative and reliable calibration standard for dual-color fluorescence cross-correlation spectroscopy. ChemPhysChem 2018, 19, 3436-3444.
(3) Schwille, P., Cross-correlation analysis in fcs. In fluorescence correlation spectroscopy. 1st ed.; Springer: 2001; Vol. 65.
(4) Bacia, K.; Petrasek, Z.; Schwille, P. Correcting for spectral cross-talk in dual-color fluorescence cross-correlation spectroscopy. ChemPhysChem 2012, 13, 1221-1231.
(5) Jejoong, Y.; N, S. A.; Chen-Yu, L.; Aleksei, A., CaDNAno to pdb file converter. 2014.
(6) Abagyan, R.; Totrov, M.; Kuznetsov, D. Icm-a new method for protein modeling and design: Applications to docking and structure prediction from the distorted native conformation. J. Comput. Chem. 1994, 15, 488-506.
(7) Vanommeslaeghe, K.; Hatcher, E.; Acharya, C.; Kundu, S.; Zhong, S.; Shim, J.; Darian, E.; Guvench, O.; Lopes, P.; Vorobyov, I.; Mackerell, A. D. Charmm general force field: A force field for drug-like molecules compatible with the charmm all-atom additive biological force fields. J. Comput. Chem. 2009, 31, 671-690.
(8) DeLano, W. L., Pymol I pymol.Org. 2002.
(9) Ao, J.; Ribeiro, V.; Radak, B.; Stone, J.; Gullingsrud, J.; Saam, J.; Phillips, J. Psfgen user's guide; 2020.
(10) Yoo, J.; Li, C.-Y.; Slone, S. M.; Maffeo, C.; Aksimentiev, A., A practical guide to molecular dynamics simulations of DNA origami systems. Humana Press Inc.: 2018; Vol. 1811, pp 209-229.
(11) Maffeo, C.; Bhattacharya, S.; Yoo, J.; Wells, D.; Aksimentiev, A. Modeling and simulation of ion channels. Chem. Rev. 2012, 112, 6250-6284.
(12) Humphrey, W.; Dalke, A.; Schulten, K. Vmd: Visual molecular dynamics. J. Mol. Graph. Model. 1996, 14, 33-38.
(13) Kessel, A.; Ben-Tal, N.; May, S. Interactions of cholesterol with lipid bilayers: The preferred configuration and fluctuations. Biophys. J. 2001, 81, 643-658.
(14) Aksimentiev, A.; Sotomayor, M.; Wells, D.; Huang, Z. Membrane proteins tutorial. Theoretical and Computational Biophysics Group, University of Illinois at Urbana-Champaign 2009.
(15) Davidchack, R. L.; Handel, R.; Tretyakov, M. V. Langevin thermostat for rigid body dynamics. J. Chem. Phys. 2009, 130, 234101-234101.
(16) Feller, S. E.; Zhang, Y.; Pastor, R. W.; Brooks, B. R. Constant pressure molecular dynamics simulation: The langevin piston method. J. Chem. Phys. 1995, 103, 4613-4621.
(17) Phillips, J. C.; Braun, R.; Wang, W.; Gumbart, J.; Tajkhorshid, E.; Villa, E.; Chipot, C.; Skeel, R. D.; Kale, L.; Schulten, K. Scalable molecular dynamics with namd. J. Comput. Chem. 2005, 26, 1781-1802.
(18) Essmann, U.; Perera, L.; Berkowitz, M. L.; Darden, T.; Lee, H.; Pedersen, L. G. A smooth particle mesh ewald method. J. Chem. Phys. 1995, 103, 8577-8593.
(19) Miyamoto, S.; Kollman, P. A. Settle—an analytical version of the shake and rattle algorithm for rigid water models. J. Comput. Chem. 1992, 13, 952-962.
(20) Darden, T.; York, D.; Pedersen, L. Particle mesh ewald: An n·log(n) method for ewald sums in large systems. J. Chem. Phys. 1993, 98, 10089-10092.
(21) Makov, G.; Payne, M. C. Periodic boundary conditions in ab initio calculations. Phys. Rev. B 1995, 51, 4014-4022.
(22) Van Der Spoel, D.; Lindahl, E.; Hess, B.; Groenhof, G.; Mark, A. E.; Berendsen, H. J. C. Gromacs: Fast, flexible, and free. J. Comput. Chem. 2005, 26, 1701-1718.
(23) Ginestet, C. Ggplot2: Elegant graphics for data analysis. J. Royal Stat. Society-Series A 2011, 174, 245-246.
(24) Racine, J. S. Rstudio: A platform-independent ide for r and sweave. J. Appl. Econ. 2012, 27, 167-172.
(25) Ramanathan, A.; Agarwal, P. K. Computational identification of slow conformational fluctuations in proteins. J. Phys. Chem. B 2009, 113, 16669-16680.
(26) Daura, X.; Gademann, K.; Jaun, B.; Seebach, D.; van Gunsteren, W. F.; Mark, A. E. Peptide folding: When simulation meets experiment. Ang. Chem. Int. Ed. 1999, 38, 236-240.
(27) Smart, O. S.; Neduvelil, J. G.; Wang, X.; Wallace, B. A.; Sansom, M. S. P. Hole: A program for the analysis of the pore dimensions of ion channel structural models. J. Mol. Graph. 1996, 14, 354-360.
(28) Giorgino, T. Computing 1-d atomic densities in macromolecular simulations: The density profile tool for vmd. Comput. Phys. Commun. 2014, 185, 317-322.
(29) Guixn-Gonzalez, R.; Rodriguez-Espigares, I.; Ramirez-Anguita, J. M.; Carrió-Gaspar, P.; Martinez-Seara, H.; Giorgino, T.; Selent, J. Membplugin: Studying membrane complexity in vmd. Bioinformatics 2014, 30, 1478-1480.
(30) Lantzsch, G.; Binder, H.; Heerklotz, H. Surface area per molecule in lipid/c12e n membranes as seen by fluorescence resonance energy transfer. J. Fluoresc. 1994, 4, 339-343.

### GENERAL REFERENCES FOR EXAMPLE

(1) Yang, N. J.; Hinner, M. J., Getting across the cell membrane: An overview for small molecules, peptides, and proteins. Gautier, A.; Hinner, M. J., Eds. 2015; Vol. 1266, pp 29-53.
(2) Hille, B., Ion channels of excitable membranes. 3rd ed.; Sinauer Associates: 2001.
(3) Grecco, H. E.; Schmick, M.; Bastiaens, P. I. H. Signaling from the living plasma membrane. Cell 2011, 144, 897-909.
(4) Fletcher, D. A.; Mullins, R. D. Cell mechanics and the cytoskeleton. Nature 2010, 463, 485-492.
(5) Jain, M.; Koren, S.; Miga, K. H.; Quick, J.; Rand, A. C.; Sasani, T. A.; Tyson, J. R.; Beggs, A. D.; Dilthey, A. T.; Fiddes, I. T.; Malla, S.; Marriott, H.; Nieto, T.; O'Grady, J.; Olsen, H. E.; Pedersen, B. S.; Rhie, A.; Richardson, H.; Quinlan, A. R.; Snutch, T. P.; Tee, L.; Paten, B.; Phillippy, A. M.; Simpson, J. T.; Loman, N. J.; Loose, M. Nanopore sequencing and assembly of a human genome with ultra-long reads. Nat. Biotechnol. 2018, 36, 338-345.
(6) Bull, R. A.; Adikari, T. N.; Ferguson, J. M.; Hammond, J. M.; Stevanovski, I.; Beukers, A. G.; Naing, Z.; Yeang, M.; Verich, A.; Gamaarachchi, H.; Kim, K. W.; Luciani, F.; Stelzer-Braid, S.; Eden, J.-S.; Rawlinson, W. D.; van Hal, S. J.; Deveson, I. W. Analytical validity of nanopore sequencing for rapid sars-cov-2 genome analysis. Nat. Commun. 2020, 11, 6272-6272.
(7) Van der Verren, S. E.; Van Gerven, N.; Jonckheere, W.; Hambley, R.; Singh, P.; Kilgour, J.; Jordan, M.; Wallace, E. J.; Jayasinghe, L.; Remaut, H. A dual-constriction biological nanopore resolves homonucleotide sequences with high fidelity. Nat. Biotechnol. 2020, 38, 1415-1420.
(8) Shi, W.; Friedman, A. K.; Baker, L. A. Nanopore sensing. Anal. Chem. 2017, 89, 157-188.
(9) Ouldali, H.; Sarthak, K.; Ensslen, T.; Piguet, F.; Manivet, P.; Pelta, J.; Behrends, J. C.; Aksimentiev, A.; Oukhaled, A. Electrical recognition of the twenty proteinogenic amino acids using an aerolysin nanopore. Nat. Nanotechnol. 2020, 38, 176-181.
(10) Qing, Y.; Tamagaki-Asahina, H.; lonescu, S. A.; Liu, M. D.; Bayley, H. Catalytic site-selective substrate processing within a tubular nanoreactor. Nat. Nanotechnol. 2019, 14, 1135-1142.
(11) Galenkamp, N. S.; Biesemans, A.; Maglia, G. Directional conformer exchange in dihydrofolate reductase revealed by single-molecule nanopore recordings. Nat. Chem. 2020, 12, 481-488.
(12) Thakur, A. K.; Movileanu, L. Real-time measurement of protein-protein interactions at single-molecule resolution using a biological nanopore. Nat. Biotechnol. 2019, 37, 96-101.
(13) Xu, C.; Lu, P.; Gamal El-Din, T. M.; Pei, X. Y.; Johnson, M. C.; Uyeda, A.; Bick, M. J.; Xu, Q.; Jiang, D.; Bai, H.; Reggiano, G.; Hsia, Y.; Brunette, T. J.; Dou, J.; Ma, D.; Lynch, E. M.; Boyken, S. E.; Huang, P. S.; Stewart, L.; DiMaio, F.; Kollman, J. M.; Luisi, B. F.; Matsuura, T.; Catterall, W. A.; Baker, D. Computational design of transmembrane pores. Nature 2020, 585, 129-134.
(14) Mahendran, K. R.; Niitsu, A.; Kong, L.; Thomson, A. R.; Sessions, R. B.; Woolfson, D. N.; Bayley, H. A monodisperse transmembrane alpha-helical peptide barrel. Nat. Chem. 2017, 9, 411-419.
(15) Joh, N. H.; Wang, T.; Bhate, M. P.; Acharya, R.; Wu, Y.; Grabe, M.; Hong, M.; Grigoryan, G.; DeGrado, W. F. De novo design of a transmembrane Zn(2)(+)-transporting four-helix bundle. Science 2014, 346, 1520-1524.
(16) Lu, P.; Min, D.; DiMaio, F.; Wei, K. Y.; Vahey, M. D.; Boyken, S. E.; Chen, Z.; Fallas, J. A.; Ueda, G.; Sheffler, W.; Mulligan, V. K.; Xu, W.; Bowie, J. U.; Baker, D. Accurate computational design of multipass transmembrane proteins. Science 2018, 359, 1042-1046.
(17) Tripathi, P.; Shuai, L.; Joshi, H.; Yamazaki, H.; Fowle, W. H.; Aksimentiev, A.; Fenniri, H.; Wanunu, M. Rosette nanotube porins as ion selective transporters and single-molecule sensors. J. Am. Chem. Soc. 2020, 142, 1680-1685.
(18) Sakai, N.; Matile, S. Synthetic ion channels. Langmuir 2013, 29, 9031-9040.
(19) Litvinchuk, S.; Tanaka, H.; Miyatake, T.; Pasini, D.; Tanaka, T.; Bollot, G.; Mareda, J.; Matile, S. Synthetic pores with reactive signal amplifiers as artificial tongues. Nat. Mater. 2007, 6, 576-580.
(20) Howorka, S. Building membrane nanopores. Nat. Nanotechnol. 2017, 12, 619-630.
(21) Xue, L.; Yamazaki, H.; Ren, R.; Wanunu, M.; Ivanov, A. P.; Edel, J. B. Solid-state nanopore sensors. Nat. Rev. Mater. 2020, 5, 931-951.
(22) Wei, R. S.; Gatterdam, V.; Wieneke, R.; Tampe, R.; Rant, U. Stochastic sensing of proteins with receptor-modified solid-state nanopores. Nat. Nanotechnol. 2012, 7, 257-263.
(23) Varongchayakul, N.; Song, J.; Meller, A.; Grinstaff, M. W. Single-molecule protein sensing in a nanopore: A tutorial. Chem. Soc. Rev. 2018, 47, 8512-8524.
(24) Rothemund, P. W. Folding DNA to create nanoscale shapes and patterns. Nature 2006, 440, 297-302.
(25) Seeman, N. C.; Sleiman, H. F. DNA nanotechnology. Nat. Rev. Mater. 2017, 3, 17068.
(26) Hong, F.; Zhang, F.; Liu, Y.; Yan, H. DNA origami: Scaffolds for creating higher order structures. Chem. Rev. 2017, 117, 12584-12640.
(27) Praetorius, F.; Kick, B.; Behler, K. L.; Honemann, M. N.; Weuster-Botz, D.; Dietz, H. Biotechnological mass production of DNA origami. Nature 2017, 552, 84-87.
(28) Sacca, B.; Niemeyer, C. M. DNA origami: The art of folding DNA. Angew. Chem. Int. Ed. 2012, 51, 58-66.
(29) Zhang, Z.; Yang, Y.; Pincet, F.; Llaguno, M.; Lin, C. Placing and shaping liposomes with reconfigurable DNA nanocages. Nat. Chem. 2017, 9, 653-659.
(30) Yang, Y. R.; Liu, Y.; Yan, H. DNA nanostructures as programmable biomolecular scaffolds. Bioconj. Chem. 2015, 26, 1381-1395.
(31) Langecker, M.; Arnaut, V.; Martin, T. G.; List, J.; Renner, S.; Mayer, M.; Dietz, H.; Simmel, F. C. Synthetic lipid membrane channels formed by designed DNA nanostructures. Science 2012, 338, 932-936.
(32) Burns, J. R.; Seifert, A.; Fertig, N.; Howorka, S. A biomimetic DNA-based channel for the ligand-controlled transport of charged molecular cargo across a biological membrane. Nat. Nanotechnol. 2016, 11, 152-156.
(33) Burns, J.; Stulz, E.; Howorka, S. Self-assembled DNA nanopores that span lipid bilayers. Nano Lett. 2013, 13, 2351-2356.
(34) Seifert, A.; Göpfrich, K.; Burns, J. R.; Fertig, N.; Keyser, U. F.; Howorka, S. Bilayer-spanning DNA nanopores with voltage-switching between open and closed state. ACS Nano 2015, 9, 1117-1126.
(35) Chen, L.; Liang, S.; Chen, Y.; Wu, M.; Zhang, Y. Destructing the plasma membrane with activatable vesicular DNA nanopores. ACS Appl. Mater. Interfaces 2020, 12, 96-105.
(36) Gopfrich, K.; Li, C. Y.; Ricci, M.; Bhamidimarri, S. P.; Yoo, J.; Gyenes, B.; Ohmann, A.; Winterhalter, M.; Aksimentiev, A.; Keyser, U. F. Large-conductance transmembrane porin made from DNA origami. ACS Nano 2016, 10, 8207-8214.
(37) Krishnan, S.; Ziegler, D.; Arnaut, V.; Martin, T. G.; Kapsner, K.; Henneberg, K.; Bausch, A. R.; Dietz, H.; Simmel, F. C. Molecular transport through large-diameter DNA nanopores. Nat. Commun. 2016, 7, 12787.
(38) Diederichs, T.; Pugh, G.; Dorey, A.; Xing, Y.; Burns, J. R.; Nguyen, Q. H.; Tornow, M.; Tampé, R.; Howorka, S. Synthetic protein-conductive membrane nanopores built with DNA. Nat. Commun. 2019, 10, 5018.
(39) Thomsen, R. P.; Malle, M. G.; Okholm, A. H.; Krishnan, S.; Bohr, S. S.; Sorensen, R. S.; Ries, O.; Vogel, S.; Simmel, F. C.; Hatzakis, N. S.; Kjems, J. A large size-selective DNA nanopore with sensing applications. Nat. Commun. 2019, 10, 5655.
(40) Chidchob, P.; Offenbartl-Stiegert, D.; McCarthy, D.; Luo, X.; Li, J. N.; Howorka, S.; Sleiman, H. F. Spatial presentation of cholesterol units on a DNA cube as a determinant of membrane protein-mimicking functions. J. Am. Chem. Soc. 2019, 141, 1100-1108.
(41) Howorka, S. Nanotechnology. Changing of the guard. Science 2016, 352, 890-891.
(42) Gopfrich, K.; Zettl, T.; Meijering, A. E.; Hernandez-Ainsa, S.; Kocabey, S.; Liedl, T.; Keyser, U. F. DNA-tile structures induce ionic currents through lipid membranes. Nano Lett. 2015, 15, 3134-3138.
(43) Lv, C.; Gu, X.; Li, H.; Zhao, Y.; Yang, D.; Yu, W.; Han, D.; Li, J.; Tan, W. Molecular transport through a biomimetic DNA channel on live cell membranes. ACS Nano 2020, 14, 14616-14626.
(44) Maingi, V.; Burns, J. R.; Uusitalo, J. J.; Howorka, S.; Marrink, S. J.; Sansom, M. S. Stability and dynamics of membrane-spanning DNA nanopores. Nat. Commun. 2017, 8, 14784.
(45) Maingi, V.; Lelimousin, M.; Howorka, S.; Sansom, M. S. Gating-like motions and wall porosity in a DNA nanopore scaffold revealed by molecular simulations. ACS Nano 2015, 9, 11209-11217.
(46) Ohmann, A.; Li, C. Y.; Maffeo, C.; Al Nahas, K.; Baumann, K. N.; Gopfrich, K.; Yoo, J.; Keyser, U. F.; Aksimentiev, A. A synthetic enzyme built from DNA flips 10(7) lipids per second in biological membranes. Nat. Commun. 2018, 9, 2426.
(47) Gopfrich, K.; Li, C. Y.; Mames, I.; Bhamidimarri, S. P.; Ricci, M.; Yoo, J.; Mames, A.; Ohmann, A.; Winterhalter, M.; Stulz, E.; Aksimentiev, A.; Keyser, U. F. Ion channels made from a single membrane-spanning DNA duplex. Nano Lett. 2016, 16, 4665-4669.
(48) Joshi, H.; Maiti, P. K. Structure and electrical properties of DNA nanotubes embedded in lipid bilayer membranes. Nucleic Acids Res. 2018, 46, 2234-2242.
(49) Im, W.; Khalid, S. Molecular simulations of gram-negative bacterial membranes come of age. Annu. Rev. Phys. Chem. 2020, 71, 171-188.
(50) Lynch, C. I.; Rao, S.; Sansom, M. S. P. Water in nanopores and biological channels: A molecular simulation perspective. Chem. Rev. 2020, 120, 10298-10335.
(51) Lanphere, C.; Arnott, P. M.; Jones, S. F.; Korlova, K.; Howorka, S. A biomimetic DNA-based membrane gate for protein-controlled transport of cytotoxic drugs. Angew. Chem. Int. Ed. 2021, 60, 1903-1908.
(52) Arnott, P. M.; Howorka, S. A temperature-gated nanovalve self-assembled from DNA to control molecular transport across membranes. ACS Nano 2019, 13, 3334-3340.
(53) Schaffter, S. W.; Schneider, J.; Agrawal, D. K.; Pacella, M. S.; Rothchild, E.; Murphy, T.; Schulman, R. Reconfiguring DNA nanotube architectures via selective regulation of terminating structures. ACS Nano 2020, 14, 13451-13462.
(54) Yurke, B.; Turberfield, A. J.; Mills, A. P., Jr.; Simmel, F. C.; Neumann, J. L. A DNA-fuelled molecular machine made of DNA. Nature 2000, 406, 605-608.
(55) Schwille, P., Cross-correlation analysis in FCS. In fluorescence correlation spectroscopy. 1st ed.; Springer: 2001; Vol. 65.
(56) Santos, J.; Gracia, P.; Navarro, S.; Pena-Diaz, S.; Pujols, J.; Cremades, N.; Pallares, I.; Ventura, S. Alpha-helical peptidic scaffolds to target alpha-synuclein toxic species with nanomolar affinity. Nat. Commun. 2021, 12, 3752.
(57) Khawaja, A.; Itoh, Y.; Remes, C.; Spahr, H.; Yukhnovets, O.; Hofig, H.; Amunts, A.; Rorbach, J. Distinct pre-initiation steps in human mitochondrial translation. Nat. Commun. 2020, 11, 2932.
(58) Yu, F.; Yao, D.; Knoll, W. Oligonucleotide hybridization studied by a surface plasmon diffraction sensor (SPDS). Nucleic Acids Res. 2004, 32, e75.
(59) Zhang, J. X.; Fang, J. Z.; Duan, W.; Wu, L. R.; Zhang, A. W.; Dalchau, N.; Yordanov, B.; Petersen, R.; Phillips, A.; Zhang, D. Y. Predicting DNA hybridization kinetics from sequence. Nat. Chemistry 2018, 10, 91-98.
(60) Wyer, J. A.; Kristensen, M. B.; Jones, N. C.; Hoffmann, S. V.; Nielsen, S. B. Kinetics of DNA duplex formation: A-tracts versus attracts. Phys. Chem. Chem. Phys. 2014, 16, 18827-18839.
(61) Howorka, S.; Movileanu, L.; Braha, O.; Bayley, H. Kinetics of duplex formation for individual DNA strands within a single protein nanopore. Proc. Natl. Acad. Sci. USA 2001, 98, 12996-13001.
(62) Dupuis, N. F.; Holmstrom, E. D.; Nesbitt, D. J. Single-molecule kinetics reveal cation-promoted DNA duplex formation through ordering of single-stranded helices. Biophys. J. 2013, 105, 756-766.
(63) Jungmann, R.; Steinhauer, C.; Scheible, M.; Kuzyk, A.; Tinnefeld, P.; Simmel, F. C. Single-molecule kinetics and super-resolution microscopy by fluorescence imaging of transient binding on DNA origami. Nano Lett. 2010, 10, 4756-4761.
(64) Gao, Y.; Wolf, L. K.; Georgiadis, R. M. Secondary structure effects on DNA hybridization kinetics: A solution versus surface comparison. Nucleic Acids Res. 2006, 34, 3370-3377.
(65) Göpfrich, K.; Zettl, T.; Meijering, A. E. C.; Hernandez-Ainsa, S.; Kocabey, S.; Liedl, T.; Keyser, U. F. DNA-tile structures induce ionic currents through lipid membranes. Nano Lett. 2015, 15, 3134-3138.
(66) Michel, W.; Mai, T.; Naiser, T.; Ott, A. Optical study of DNA surface hybridization reveals DNA surface density as a key parameter for microarray hybridization kinetics. Biophys. J. 2007, 92, 999-1004.
(67) Pappaert, K.; Van Hummelen, P.; Vanderhoeven, J.; Baron, G. V.; Desmet, G. Diffusion-reaction modelling of DNA hybridization kinetics on biochips. Chem. Eng. Sci. 2003, 58, 4921-4930.
(68) Zhang, D. Y.; Chen, S. X.; Yin, P. Optimizing the specificity of nucleic acid hybridization. Nat. Chemistry 2012, 4, 208-214.
(69) Peterson, A. W.; Heaton, R. J.; Georgiadis, R. M. The effect of surface probe density on DNA hybridization. Nucleic Acids Res. 2001, 29, 5163-5168.
(70) Burns, J. R.; Howorka, S. Defined bilayer interactions of DNA nanopores revealed with a nuclease-based nanoprobe strategy. ACS Nano 2018, 12, 3263-3271.
(71) Engman, K. C.; Sandin, P.; Osborne, S.; Brown, T.; Billeter, M.; Lincoln, P.; Nordén, B.; Albinsson, B.; Wilhelmsson, L. M. DNA adopts normal b-form upon incorporation of highly fluorescent DNA base analogue tc: NMR structure and UV-vis spectroscopy characterization. Nucleic Acids Res. 2004, 32, 5087-5095.
(72) Kypr, J.; Kejnovska, I.; Renciuk, D.; Vorlickova, M. Circular dichroism and conformational polymorphism of DNA. Nucleic Acids Res. 2009, 37, 1713-1725.
(73) Banchelli, M.; Betti, F.; Berti, D.; Caminati, G.; Bombelli, F. B.; Brown, T.; Wilhelmsson, L. M.; Nordén, B.; Baglioni, P. Phospholipid membranes decorated by cholesterol-based oligonucleotides as soft hybrid nanostructures. J. Phys. Chem. B 2008, 112, 10942-10952.
(74) Lundberg, E. P.; Feng, B.; Saeid Mohammadi, A.; Wilhelmsson, L. M.; Nordén, B. Controlling and monitoring orientation of DNA nanoconstructs on lipid surfaces. Langmuir 2013, 29, 285-293.
(75) Ardhammar, M.; Lincoln, P.; Norden, B. Nonlinear partial differential equations and applications: Invisible liposomes: Refractive index matching with sucrose enables flow dichroism assessment of peptide orientation in lipid vesicle membrane. Proc. Natl. Acad. Sci. USA 2002, 99, 15313-15317.
(76) Rodger, A.; Dorrington, G.; Ang, D. L. Linear dichroism as a probe of molecular structure and interactions. The Analyst 2016, 141, 6490-6498.
(77) Ohmann, A.; Gopfrich, K.; Joshi, H.; Thompson, R. F.; Sobota, D.; Ranson, N. A.; Aksimentiev, A.; Keyser, U. F. Controlling aggregation of cholesterol-modified DNA nanostructures. Nucleic Acids Res. 2019, 47, 11441-11451.
(78) Walsh, A. S.; Yin, H.; Erben, C. M.; Wood, M. J.; Turberfield, A. J. DNA cage delivery to mammalian cells. ACS Nano 2011, 5, 5427-5432.
(79) Garcia-Fandino, R.; Pineiro, A.; Trick, J. L.; Sansom, M. S. Lipid bilayer membrane perturbation by embedded nanopores: A simulation study. ACS Nano 2016, 10, 3693-3701.
(80) Smart, O. S.; Neduvelil, J. G.; Wang, X.; Wallace, B. A.; Sansom, M. S. P. Hole: A program for the analysis of the pore dimensions of ion channel structural models. J. Mol. Graph. 1996, 14, 354-360.
(81) Lanphere, C.; Offenbartl-Stiegert, D.; Dorey, A.; Pugh, G.; Georgiou, E.; Xing, Y.; Burns, J. R.; Howorka, S. Design, assembly, and characterization of membrane-spanning DNA nanopores. Nat. Protoc. 2021, 16, 86-130.
(82) Shannon, R. D. Revised effective ionic radii and systematic studies of interatomic distances in halides and chalcogenides. Acta Crystallogr. A 1976, 32, 751-767.
(83) Berendes, R.; Burger, A.; Voges, D.; Demange, P.; Huber, R. Calcium influx through annexin v ion channels into large unilamellar vesicles measured with Fura-2. FEBS Letters 1993, 317, 131-134.
(84) Hugonin, L.; Vukojević, V.; Bakalkin, G.; Graslund, A. Calcium influx into phospholipid vesicles caused by dynorphin neuropeptides. Biochim Biophys Acta 2008, 1778, 1267-1273.
(85) Song, L.; Hobaugh, M. R.; Shustak, C.; Cheley, S.; Bayley, H.; Gouaux, J. E. Structure of staphylococcal alpha-hemolysin, a heptameric transmembrane pore. Science 1996, 274, 1859-1866.
(86) Leung, C.; Hodel, A. W.; Brennan, A. J.; Lukoyanova, N.; Tran, S.; House, C. M.; Kondos, S. C.; Whisstock, J. C.; Dunstone, M. A.; Trapani, J. A.; Voskoboinik, I.; Saibil, H. R.; Hoogenboom, B. W. Real-time visualization of perforin nanopore assembly. Nat. Nanotechnol. 2017, 12, 467-473.
(87) Yoshina-Ishii, C.; Boxer, S. G. Arrays of mobile tethered vesicles on supported lipid bilayers. J. Am. Chem. Soc. 2003, 125, 3696-3697.
(88) Andersen, E. S.; Dong, M.; Nielsen, M. M.; Jahn, K.; Subramani, R.; Mamdouh, W.; Golas, M. M.; Sander, B.; Stark, H.; Oliveira, C. L.; Pedersen, J. S.; Birkedal, V.; Besenbacher, F.; Gothelf, K. V.; Kjems, J. Self-assembly of a nanoscale DNA box with a controllable lid. Nature 2009, 459, 73-76.
(89) Niemeyer, C. M. Engineering for life sciences: A fruitful collaboration enabled by chemistry. Angew. Chem. Int. Ed. 2017, 56, 1934-1935.
(90) Veetil, A. T.; Chakraborty, K.; Xiao, K.; Minter, M. R.; Sisodia, S. S.; Krishnan, Y. Cell-targetable DNA nanocapsules for spatiotemporal release of caged bioactive small molecules. Nat. Nanotechnol. 2017, 12, 1183-1189.

Although particular embodiments of the invention have been disclosed herein in detail, this has been done by way of example and for the purposes of illustration only. The aforementioned embodiments are not intended to be limiting with respect to the scope of the appended claims, which follow. For example, the choice of nucleic acid starting material is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1. A nucleic acid nanostructure comprising:
a plurality of component modules, each component module comprising a nucleic acid sequence and at least one membrane anchor, and
wherein the plurality of component modules are capable of undertaking a controlled assembly in response to an external stimulus to form the nanostructure; and
wherein the nanostructure is configured to penetrate a semifluid membrane upon or following the controlled assembly.

2. The nucleic acid nanostructure of claim 1, wherein the plurality of component modules are able to associate with a semifluid membrane prior to controlled assembly.

3. The nucleic acid nanostructure of claim 2, wherein the plurality of component modules are able to associate with a semifluid membrane following controlled assembly.

4. The nucleic acid nanostructure of claim 1, wherein the external stimulus comprises the binding of a molecule to at least one of the plurality of component modules.

5. The nucleic acid nanostructure of claim 4, wherein the external stimulus comprises a molecule selected from one or more of the group consisting of: a nucleic acid sequence; an aptamer; a small molecule; an antibody or an antibody fragment; an antibody mimetic; a peptide; a polypeptide; a polysaccharide; an oligosaccharide; a macromolecule within the size range: 1-10 kD, 1-50 kD, 1-100 kD, 10-50 kD, 10-100 kD, 20-50 kD, and 20-100 kD; a carbohydrate; a biopolymer; a toxin; a metabolite; and/or a cytokine.

6. The nucleic acid nanostructure of claim 4, wherein the external stimulus comprises a molecule selected from the group consisting of:
I. an enzyme—including a polymerase, a helicase, a gyrase, and a telomerase, as well as nucleic acid binding sub domains or derivatives thereof;
II. synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. synthetic or naturally derived affinity binding nucleic acids and nucleic acid analogues, including oligonucleotide probes, aptamers and ribozymes;
V. naturally occurring or synthetic small molecules, including drugs, fluorophores, metabolites and chemokines;
VI. an antigen or antigenic fragment; and
VII. signalling molecules and/or polypeptide receptors thereof, including binding domains of receptors, and receptor complexes.

7. The nucleic acid nanostructure of claim 2, wherein the semifluid membrane comprises a synthetic amphipathic membrane or a lipid bilayer membrane.

8. The nucleic acid nanostructure of claim 7, wherein the nanostructure is a nanopore and optionally wherein the nanopore comprises a central lumen with a minimum internal width of at least 0.2 nm, suitably at least 0.5 nm, optionally at least 0.75 nm.

9. The nucleic acid nanostructure of claim 7, wherein the nanostructure is a nanopore and optionally wherein the nanopore comprises a central lumen with a maximum internal width of at most about 20 nm, suitably at most about 10 nm, optionally at most around 5 nm.

10. The nucleic acid nanostructure of claim 1, wherein the nucleic acid nanostructure is comprised of DNA.

11. The nucleic acid nanostructure of claim 10, wherein each of the plurality of component modules comprises a DNA sequence comprises at least a portion of which comprises a double helix that defines a secondary structure and at least a portion of which comprises a single stranded sequence that defines an assembly interface.

12. The nucleic acid nanostructure of claim 11, wherein the assembly interface of each of the plurality of component modules comprises a sequence that is complementary to and is capable of hybridising with the assembly interface of another of the component modules.

13. The nucleic acid nanostructure of claim 1, wherein the membrane anchor comprises a hydrophobic molecule, suitably wherein the hydrophobic molecule comprises a cholesterol molecule, or an analogue thereof; a sterol; an alkylated phenol; a flavone; a saturated or unsaturated fatty acid; a synthetic lipid molecule; or a dodecyl-beta-D-glucoside.

14. The nucleic acid nanostructure of claim 1, wherein the nanostructure is comprised of at least two component modules.

15. The nucleic acid nanostructure of claim 1, wherein the nanostructure is comprised of at least three component modules.

16. The nucleic acid nanostructure of claim 1, wherein assembly of the nanostructure and/or components thereof is via DNA origami techniques.

17. A nucleic acid nanostructure comprising:
a plurality of component modules, each component module comprising a deoxyribonucleic acid (DNA) sequence, wherein the DNA sequence comprises at least a portion of double helix that defines a secondary structure and at least a portion of single stranded sequence that defines an assembly interface, and at least one hydrophobic anchor; and
a plurality of single stranded nucleic acid lock sequences that are capable of hybridising with the single stranded sequence of the assembly interface;
wherein the component modules are able to associate and interact with a surface of a semifluid membrane via the anchor, and
wherein the modules are capable of undertaking a controlled assembly in response to an externally applied stimulus that initiates disassociation of the plurality of lock sequences to reveal the assembly interfaces thereby allowing the plurality of component modules to associate and thereby form a nanostructure.

18. The nucleic acid nanostructure of claim 17, wherein the nanostructure is configured to penetrate the semifluid membrane during or after the controlled assembly.

19. The nucleic acid nanostructure of claim 17, wherein the externally applied stimulus comprises a molecule selected from one or more of the group consisting of: a nucleic acid sequence; an aptamer; a small molecule; an antibody or an antibody fragment; an antibody mimetic; a peptide; a polypeptide; a polysaccharide; an oligosaccharide; a macromolecule within the size range: 1-10 kD, 1-50 kD, 1-100 kD, 10-50 kD, 10-100 kD, 20-50 kD, and 20-100 kD; a carbohydrate; a biopolymer; a toxin; a metabolite; and/or a cytokine.

20. The nucleic acid nanostructure of claim 17, wherein the externally applied stimulus comprises a molecule selected from the group consisting of:
I. an enzyme—including a polymerase, a helicase, a gyrase, and a telomerase, as well as nucleic acid binding sub domains or derivatives thereof;
II. synthetic or naturally derived affinity binding proteins and peptides—including affimers, antigen binding microproteins, engineered multiple repeat proteins, ankyrin binding domains, lactoferrins, cathelicidins, ficolins, collagenous lectins, T-cell receptor domains and defensins;
III. an antibody—including polyclonal, monoclonal, humanized and camelid antibodies, or antigen binding fragments and derivatives thereof, including Fab, scFv, Bis-scFv, VH, VL, V-NAR, VhH or any other antigen-binding single domain antibody fragment;
IV. synthetic or naturally derived affinity binding nucleic acids and nucleic acid analogues, including oligonucleotide probes, aptamers and ribozymes;
V. naturally occurring or synthetic small molecules, including drugs, fluorophores, metabolites and chemokines;
VI. an antigen or antigenic fragment; and
VII. signalling molecules and/or polypeptide receptors thereof, including binding domains of receptors, and receptor complexes.

21. The nucleic acid nanostructure of claim 17, wherein the semifluid membrane comprises a synthetic amphipathic membrane or a lipid bilayer membrane.

22. The nucleic acid nanostructure of claim 17, wherein the nanostructure that is formed following the controlled assembly is a membrane-spanning nanopore.

23. The nucleic acid nanostructure of claim 22, wherein the nanopore comprises a central lumen with a minimum internal width of at least 0.2 nm, suitably at least 0.5 nm, optionally at least 0.75 nm.

24. The nucleic acid nanostructure of claim 22, wherein the nanopore comprises a central lumen with a maximum internal width of at most about 20 nm, suitably at most about 10 nm, optionally at most around 5 nm.

25. The nucleic acid nanostructure of claim 17, wherein the assembly interface of each of the plurality of component modules comprises a sequence that is complementary to and is capable of hybridising with the assembly interface of another of the component modules.

26. The nucleic acid nanostructure of claim 17, wherein the hydrophobic comprises: a sterol; an alkylated phenol; a flavone; a saturated or unsaturated fatty acid; or a synthetic lipid molecule.

27. The nucleic acid nanostructure of claim 26, wherein:
the sterols are selected from the group consisting of: cholesterol; derivatives of cholesterol; phytosterol; ergosterol; and bile acid;
the alkylated phenols are selected from the group consisting of: methylated phenols; dolichols and tocopherols;
the flavones are selected from the group consisting of: flavanone containing compounds; and 6-hydroxyflavone;
the saturated and unsaturated fatty acids are selected from the group consisting of: derivatives of lauric acid; oleic acid; linoleic acid; and palmitic acids; and/or
the synthetic lipid molecule is dodecyl-beta-D-glucoside.

28. A semifluid membrane onto which is associated a nanostructure of claim 1.

29. A sensor device, wherein the sensor comprises a nucleic acid nanostructure as defined in claim 1.

30. The sensor device of claim 29, wherein the nucleic acid nanostructure is a membrane spanning nanopore.
